(19) United States
(12) Patent Application Publication
(10) Pub. No.: US 2017/0234874 A1
(43) Pub. Date: Aug. 17, 2017

(54) INTEGRATED VISUAL MORPHOLOGY AND CELL PROTEIN EXPRESSION USING RESONANCE-LIGHT SCATTERING

(71) Applicant: Clearbridge BioPhotonics Pte Ltd., Singapore (SG)

(72) Inventors: Thomas H. Adams, Rancho Santa Fe, CA (US); Stephen Roman Fait, Carlsbad, CA (US); Eric Scott McCampbell, Carlsbad, CA (US); Michelle Brooke McCampbell, Rancho Santa Fe, CA (US); Edward Jablonski, Escondido, CA (US); Robert Earl Klem, Rancho Santa Fe, CA (US)

(21) Appl. No.: 15/286,340

(22) Filed: Oct. 5, 2016

Related U.S. Application Data
(60) Provisional application No. 62/238,605, filed on Oct. 7, 2015.

(51) Int. Cl. G01N 33/569; G01N 15/14; A61B 5/00; G01N 21/552

(52) U.S. Cl. CPC G01N 33/56966; G01N 21/553; G01N 15/1456; A61B 5/0082; G06T 7/0012

(57) ABSTRACT

The invention relates to detecting cell biomarker signatures and integrated cell biomarker-morphological profiles by detecting resonance-light scattering of functionalized nanoparticles.

### RELATED APPLICATIONS

This application claims the benefit of U.S. Provisional Application Ser. No. 62/238,605, filed on Oct. 7, 2015, the entire contents of which is hereby incorporated be reference in its entirety.

### FIELD

The presently disclosed subject matter relates to compositions and methods for integrated visual morphology and cell protein expression analysis.

### INTRODUCTION AND SUMMARY OF THE INVENTION

Cellular analysis is an important tool in histopathology to aid in diagnosing the medical condition of a subject. Two pathological tools clinicians use for cellular analysis is the morphological analysis of cell samples using microscopy and cellular biomarker detection using a method such as flow cytometry. Morphological analysis of cells samples involves visually identifying features or characteristics of a cell and associating those features or characteristics with known disease or condition states of a cell. Often, morphological analysis is insufficient to diagnose the disease state or condition of a cell, and tissues or samples are analyzed for the presence of biomarkers associated with known disease or conditions. While biomarkers can be any molecule that indicates a biological state, they are most often peptides or proteins. These peptides or proteins are involved in many roles in the body, including intercellular signaling and metabolism. Cell signaling and metabolism refer to the mechanisms behind common disease states and the associated markers may be used for measuring and monitoring such observables as disease progression and drug response.

Often, biomarkers are designated by their CD nomenclature. CD (cluster of differentiation) molecules are cell surface biomarkers useful for the identification and characterization of leukocytes. The CD nomenclature was developed and is maintained through the HLDA (Human Leukocyte Differentiation Antigens) working group. Identifying various combinations of CD molecules on a cell surface is often used for the classification of cell types and surface molecules which are targets for the immunophenotyping of cells. (Chan, J. et al, Histopathology 12 (5): 461-480 (1988)).

Resonance light scattering is a physical phenomenon wherein a particle with a diameter less than the wavelength of incident light exhibits a surface plasmon wave around the particle and said wave becomes coherent to the circumference of the particle. Particle electrons resonate in phase with the incident light forming an electromagnetic dipole that emits energy as scattered light. The wavelength of the reflected (scattered) light is a function of the composition, shape, and particle size. Often, the composition of the particle is a noble metal, such as gold or silver. Often, the size of the particle is below the wavelength of white light (below 200 nm). Particles under 1000 nm in size are often referred to as “nanoparticles.”

The advantages of using particles with a wavelength less than the wavelength of light for cellular analysis using the resonant light scattering properties of the particles are: (a) the nanoparticles can be detected and imaged at magnifications as low as 10× using a simple illuminator, such as a white light illuminator, with dark field illumination, (b) the nanoparticles provide a non-bleaching signal, (c) the color of scattered light can be changed by changing nanoparticle size and/or composition for multicolor multiplexing, (d) the nanoparticles can be conjugated with biomarker-binding moieties for specific analyte detection to create functionalized nanoparticles, (e) biological samples contacted with the functionalized nanoparticles are archivable, and (f) the functionalized nanoparticles exhibit a greater range of linearity of detection when present on a cell because the particles do not self-quench. In some embodiments, the methods of this invention are useful in obtaining images of cell-functionalized nanoparticle complexes under ambient conditions which do not require use of a darkroom, in contrast to fluorescent labeling systems. In some embodiments, the samples may be viewed on a microscope in a doctor's or pathologist's office.

The present disclosure relates in some aspects to methods and compositions for detecting cell-functionalized nanoparticle binding moiety complexes useful in detecting a biomarker signature of a cell. In some aspects, the methods and compositions are also useful in detecting the biomarker-morphological profile of an imaged cell.

In some aspects of this disclosure, the nanoparticles functionalized with biomarker-binding moieties can be used for detecting functionalized nanoparticle cell complexes, which are useful, for example, for identifying and quantifying biomarkers present on cells. In some embodiments, the cells may be imaged to detect morphological features of the cells complexed with functionalized nanoparticles. In some embodiments, the functionalized nanoparticles can be contacted with the same cells analyzed by a morphological imaging analysis.

In some embodiments, the methods of this invention are useful for improving signal generation, detection limits, dynamic range, automation and/or performance characteristics of the biomarker signature assays. For example, in some embodiments, the present disclosure relates to methods for increasing the loading amount of a biomarker binding moiety onto a cell by using an external force to increase the local concentration of the functionalized nanoparticles and cells. In some aspects, the biomarker binding moiety may be a functionalized nanoparticle, or a biomarker binding moiety comprising a label other than a nanoparticle. A “functionalized nanoparticle” is a nanoparticle presenting a functional group, directly or indirectly. In some aspects, the external force may be a centrifugal, electromagnetic, or magnetic force.

In some aspects of this invention, the method of detecting functionalized nanoparticle cell complexes can comprise:
(a) providing a sample comprising cells from a subject;
(b) contacting the cells with one or a plurality of functionalized nanoparticle species, each functionalized nanoparticle species comprising a biomarker-binding moiety, wherein an external force is used to accelerate the formation of nanoparticle-cell complexes through binding of the nanoparticle species comprising a biomarker-binding moiety to its respective biomarker;
(c) adhering the functionalized nanoparticle-cell complexes to a substrate;
(d) detecting the functionalized nanoparticle cell complexes by illuminating the nanoparticle-cell complexes with evanescent light and detecting the resonant light scattering from each observed functionalized nanoparticle cell complex, to obtain a biomarker signature of each observed cell; and
(e) associating the biomarker signature of each substrate-adhered cell to a known disease, condition, or state of a cell exhibiting substantially the same or a similar biomarker signature to identify the disease, condition, or state of the cell from a subject.

In any of the methods disclosed herein, where a biomarker or biomarker morphological profile is obtained, detecting the cell-functionalized nanoparticle complexes on the imaged cell optionally includes storing the positional information for each imaged cell.

In some embodiments, the disease, condition, or state of a cell can be identified by forming and detecting complexes between the functionalized nanoparticles and cells. The method can comprise associating a biomarker signature of each substrate-adhered cell to a known disease, condition, or state of a cell exhibiting substantially the same or a similar biomarker signature to identify the disease, condition, or state of the cell from a subject.

In some embodiments, the biomarker signature of a cell can be detected in a homogeneous assay, the assay comprising the steps:
(a) providing a sample comprising cells from a subject;
(b) contacting the cells with one or a plurality of functionalized nanoparticle species, each functionalized nanoparticle species comprising a biomarker-binding moiety, and forming functionalized nanoparticle-cell complexes through binding of the nanoparticle species comprising a biomarker-binding moiety to its respective biomarker;
(c) adhering the functionalized nanoparticle-cell complexes to a substrate;
(d) illuminating the functionalized nanoparticle-cell complexes with evanescent light and detecting the resonant light scattering from each observed complexed nanoparticle, to obtain a biomarker signature of each observed cell,
   where unbound functionalized nanoparticles are not removed from the field of view.

Often, unbound species are washed from a target to reduce background noise. In some embodiments wash steps are omitted, leaving unbound functionalized nanoparticles in the field of view. This embodiment may be used in some embodiments, for example, for high throughput assays, and/or automated assays. The functionalized nanoparticles are specific to the biomarker on the cell, and can substantially contact the cell such that little to no signal is observed for the unbound functionalized nanoparticles.

In some aspects of this invention, where biomarker signatures are detected, the detecting of the resonant light scattering from each observed complexed-nanoparticle comprises imaging the cell-functionalized nanoparticle complexes in contact with a mountant. In some aspects of this invention, the mountant can comprise a solution with about the refractive index of the cells. In some aspects of this invention, the mountant can be within about 0.1 of the refractive index of cells, where the cells are fixed. In some embodiments, the refractive index of fixed cells is about 1.52, or 1.52. In some aspects, the index of refraction of the mountant is from 1.51 to 1.54. In some aspects, using a mountant having an RI within 0.1 of the refractive index of fixed cells is useful for reducing the amount of white light scatter, and obtaining better images of resonance scattering from the cell-functionalized nanoparticle complexes. In one aspect, this disclosure relates to a method for detecting functionalized nanoparticle cell complexes, the method comprising:
(a) providing a sample comprising cells from a subject;
(b) contacting cells which have been fixed with one or a plurality of functionalized nanoparticle species, each functionalized nanoparticle species comprising a biomarker-binding moiety, and forming nanoparticle-cell complexes through binding of the nanoparticle species comprising a biomarker-binding moiety to its respective biomarker;
(c) adhering the nanoparticle-cell complexes to a substrate, wherein the adhered nanoparticle-cell complexes are placed in contact with a mountant, wherein the refractive index of the mountant is within about 0.1 of the refractice index of the fixed cells;
(d) detecting the functionalized nanoparticle cell complexes by illuminating the nanoparticle-cell complexes with evanescent light and detecting the resonant light scattering from each observed functionalized nanoparticle cell complex, to obtain a biomarker signature of each observed cell; and
(e) associating the biomarker signature of each substrate-adhered cell to a known disease, condition, or state of a cell exhibiting substantially the same biomarker signature to identify the disease, condition, or state of the cell from a subject.
   In some aspects, the mountant may have a refractive index of about 1.52.

In some aspects, the ability to use the compositions and methods of this disclosure to associate the biomarker signature of an individual cell, and in some embodiments, with its morphological image/features greatly enhances the ability to diagnose and monitor abnormal conditions or disorders.

As set forth herein, the inventors have surprisingly determined that nanoparticles functionalized with biomarker-binding moieties can be used for detecting cell-functionalized nanoparticle complexes and identifying and quantifying biomarkers present on imaged cells for example, when the functionalized nanoparticles are contacted with the same cells analyzed by a morphological imaging analysis. The ability to use the compositions and methods of this disclosure to associate the biomarker signature of an individual cell with its morphological image/features greatly enhances the ability to diagnose and monitor abnormal conditions or disorders.

In one aspect, this disclosure relates to compositions and methods for obtaining a biomarker signature for an imaged cell, which is used, in some embodiments, in combination with detected morphological features of the cell obtained from imaging the cell. In some aspects, compositions comprising functionalized nanoparticle species, each comprising a specific biomarker-binding moiety, are used to detect the biomarker signature of the imaged cell. In some aspects of this disclosure, combinations of such compositions are made or used in the methods of this disclosure. The combinations and kits comprising the combinations can be mixtures of such compositions, or may comprise compositions segregated before use.

In one aspect the method comprises the steps of:
(a) providing a sample comprising cells from a subject;
(b) contacting the cells with one or a plurality of functionalized nanoparticle species, each functionalized nanoparticle species comprising a biomarker-binding moiety, and forming nanoparticle-cell complexes through binding of the nanoparticle species comprising a biomarker-binding moiety to its respective biomarker on the cell;
(c) adhering the functionalized nanoparticle-cell complexes to a substrate;
(d) illuminating the functionalized nanoparticle-cell complexes with evanescent light and detecting the resonant light scattering from each observed complexed functionalized nanoparticle, to obtain a biomarker signature of each observed cell;
(e) contacting the substrate-adhered cells with an optical contrast agent;
(f) imaging morphological features of contacted cells; and
(g) associating the morphological features of the contacted cells with the biomarker signature of each substrate-adhered cell to detect the biomarker-morphological profile of each cell.

In some aspects, biomarker-morphological profiles can be used to obtain a diagnostic concordance between a reference cell biomarker signature and a disease, disorder, condition or state of a reference subject. In some embodiments, a concordance database of biomarker signatures with diseases, disorders, conditions or states of diagnosed subjects can be obtained. A diagnostic concordance includes an association based on an association between a reference biomarker signature and/or a reference biomarker-morphological profile, and a disease, disorder, condition or state of the reference subject. In some embodiments the diagnostic concordance or association, may be made independent of any treatment decision, for example, using data obtained from an autopsy and/or based on archived tissue samples and patient records.

This disclosure relates, in some aspects, to a method for detecting functionalized nanoparticle cell complexes to obtain a biomarker signature, the method comprising:
(a) providing a sample comprising cells from a subject;
(b) contacting the cells with one or a plurality of functionalized nanoparticle species, each functionalized nanoparticle species comprising a biomarker-binding moiety, and forming nanoparticle-cell complexes through binding of the functionalized nanoparticle species comprising a biomarker-binding moiety to its respective biomarker;
(c) adhering the functionalized nanoparticle-cell complexes to a substrate;
(d) illuminating the functionalized nanoparticle-cell complexes with evanescent light and detecting the resonant light scattering from each observed complexed nanoparticle, to obtain a biomarker signature of each observed cell; and
(e) associating the biomarker signature of an imaged cell from the subject with a biomarker signature of a reference cell exhibiting substantially the same biomarker signature as the imaged cell biomarker signature, wherein a diagnostic concordance has been established between the reference cell biomarker signature and a disease, disorder, condition or state of the reference subject.

In some aspects of this disclosure, the sample can be a biological sample. For example, in some embodiments, the sample may be any sample containing cells. In some embodiments, the sample may be from blood, bone marrow, a fine needle aspirate, or tissue. The tissue sample can be obtained, for example, from a biopsy. In some embodiments the tissue sample may be obtained from a FFPE (formalin-fixed, paraffin-embedded) tissue sample. In some aspects of this disclosure, a biological sample may be processed. For example, the sample may comprise white blood cells. In some embodiments, at least 50% of the red blood cells are removed before contacting the cells with the plurality of functionalized nanoparticle species.

In some aspects of this disclosure, the cell may be alive, fixed and/or substantially intact. In some embodiments, the detected cells interrogated can be the same type or different types. When the cells are different types, they may be of different tissue or tumor origin, different stages of cancer progression, metastatic and non-metastatic cancer cells, and may comprise infectious agents or cells, infected cells, and uninfected cells, or express different levels, types, variants, mutants, forms, and/or post-translationally modified forms of biomarkers found on normal or reference cells. In some embodiments, the different cells may exhibit different pathologies, and/or different morphologies from normal or reference cells.

In some aspects of this disclosure, the cell is fixed with a fixing agent. The fixing agent may be, for example, formaldehyde, glutaraldehyde, or another cross-linking agent. In other embodiments water-soluble preservatives, for example, methyl or propyl paraben, dimethylolurea, sorbic acid, 2-pyridinethiol-1-oxide, or potassium sorbate may be used. In some embodiments the cell can be permeabilized by surfactants.

In some aspects of this disclosure, the functionalized nanoparticle cell complexes adhered to a substrate can be placed in contact with a mountant. The volume of the mountant can be from about 2 microliters to about 15 microliters.

In some aspects of this disclosure, the detected biomarker can be present on the cell surface, within the cell, or both on the surface and within the cell. In some embodiments the biomarker in the cell may be present in or on one or more cellular features, for example, the cytosol, the nucleus, the nuclear membrane, nucleoli, the endoplasmic reticulum, Golgi apparatus, mitochondria, or other cellular structure, compartment, or feature.

In some aspects of this disclosure, the detected biomarker can be a biomolecule identified by the Cluster Determinant antigen (CD) and or other molecules/antigenic sites. For example, the biomarker can include or exclude any of the from the following biomarkers: CD1, CD2, CD3, CD4, CD5, CD6, CD7, CD8, CD9, CD10, CD11a, CD11b, CD11c, CD13, CD14, CD15, CD16, CD19, CD20, CD21, CD22, CD23, CD25, CD30, CD33, CD34, CD38, CD41, C43, CD45, CD56, CD57, CD58, CD61, CD64, C71, CD79a, CD99, CD103, CD117, CD123, CD138, CD138, CD163, CD235a, HLA-DR, Kappa, Lambda, Pax-5, BCL-2, Ki-67, ZAP-70, MPO, TdT, and FMC-7. In some embodiments, for example, the biomarker can include or exclude markers expressed by kidney cells, infectious agents, solid tumor cells, circulating tumor cells, or any other cell useful for diagnosis or prognosis. In some embodiments, for example, the biomarker can include or exclude biomarkers expressed on the surface or within kidney cells, infectious agents (e.g., bacteria or virus), solid tumor cells, or circulating tumor cells. In some embodiments, for example the biomarker may include or exclude HER2, NEU, Prostate stem cell antigen (PSCA), epithelial-specific antigen (ESA), epithelial cell adhesion molecule (EpCAM), α2β1, VEGFR-1, VEGFR-2, CD133, or AC133 antigen.

In some aspects of this disclosure, the biomarker-binding moiety can be selected from or comprise the following: an antibody or antibody fragment, nanobody, receptor fragment, DNA aptamer, DNA/RNA oligonucleotide, RNA aptamer, PNA aptamer, peptide aptamer, LNA aptamer, carbohydrate, and a lectin.

In embodiments where the biomarker binding moiety comprises an antibody, the antibody can be a monoclonal or polyclonal antibody. In some embodiments, the biomarker binding moiety may comprise an antibody fragment, ScFv, or single-domain antibody (nanobody). The biomarker binding moiety may bind to a protein, protein fragment, glycosylation moiety or pattern, or a carbohydrate. The biomarker binding moiety can include or exclude a biomarker binding moiety, e.g., an antibody or fragment thereof or other biomarker binding moiety that binds to, for example: CD1, CD2, CD3, CD4, CD5, CD6, CD7, CD8, CD9, CD10, CD11a, CD11b, CD11c, CD13, CD14, CD15, CD16, CD19, CD20, CD21, CD22, CD23, CD25, CD30, CD33, CD34, CD38, CD41, C43, CD45, CD56, CD57, CD58, CD61, CD64, C71, CD79a, CD99, CD103, CD117, CD123, CD138, CD138, CD163, CD235a, HLA-DR, Kappa, Lambda, Pax-5, BCL-2, Ki-67, ZAP-70, MPO, TdT, and FMC-7. In some embodiments, when the biomarker-binding moiety is anti-CD45, the biomarker signature obtained is the white blood cell count. The white blood cell count can be measured as a function of the mass or volume of the sample mass or volume, respectively.

In some embodiments, the optical contrast agent can be a leuco dye, cell stain, or any dye useful for imaging for morphological analysis including, for example, any dye useful for histological, cytological, cytopathological, or histopathological imaging. In some embodiments, the optical contrast agent provides visual classification and identification of cells by differentially staining cells. The leuco dye can be red leuco dye, methylene blue, crystal violet, phenolphthalein, thymolphthalein, or methylene green.

In some embodiments, the optical contrast agent can include or exclude a cell stain selected from, for example: Giemsa stain, Wright stain, Wright-Giemsa stain, May-Grünwald stain, Mallory trichrome, Periodic acid-Schiff reaction stain, Weigert's elastic stain, Heidenhain's AZAN trichrome stain, Orcein stain, Masson's trichrome, Alcian blue stain, May-Grünwald-Giemsa, van Gieson stain, Hansel stain, Reticulin Stain, Gram stain, Bielschowsky stain, Ferritin stain, Fontana-Masson stain, Hales colloidal iron stain, Pentachrome stain, Azan stain, Luxol fast blue stain, Golgi's method (reduced silver), reduced gold, chrome alum/haemotoxylin stain, Isamin blue stain, Argentaffin stains, Warthin-Starry silver stain, Nissl stain, Sudan Black and osmium stain, osmium tetroxide stain, hematoxylin stain, uranyl acetate stain, lead citrate stain, Carmine stain, safranin stain, and Ziehl-Neelsen stain.

In some embodiments, the optical contrast agent can be a dye or colorant that can include or exclude, for example: eosin Y, eosin B, azure B, pyronin G, malachite green, toluidine blue, copper phthalocyanin, alcian blue, auramine-rhodamine, acid fuschin, aniline blue, orange G, acid fuschin, neutral red, Sudan Black B, acridine orange, Oil Red O, Congo Red, Fast green FCF, Perls Prussian blue reaction, nuclear fast red, alkaline erythrocin B, and naphthalene black.

When the sample is from tissue, the optical contrast agent can be a H&E (hematoxylin and eosin) stain. In one aspect, the optical contrast agent may be suitable for supravital staining.

In some embodiments, the cells can be contacted with one or a plurality of functionalized nanoparticle species by subjecting the cells and functionalized nanoparticles to an external force to increase the local concentration of the functionalized nanoparticles and cells. The external force can be a gravitational, electric, or magnetic force. The gravitational force can be generated by centrifugation. The magnetic force can be effected by paramagnetic nanoparticles. The core of a paramagnetic functionalized nanoparticle comprises a paramagnetic region and the shell of the nanoparticle can include or exclude Ag, Au, Pt, Pd, Rh, Ro, Al, Cu, Ru, Cr, Cd, Zn, Si, Se or mixtures or alloys thereof. In some embodiments, charged polymers can be added to the cells after first providing a sample comprising cells from a subject. These methods may be useful, for example, for methods of detecting cell-biomarker binding moieties, for example, in any embodiment of detecting cell-functionalized nanoparticle complexes disclosed herein. In some embodiments the sample may be re-mixed between two or more applications of the external force. As one non-limiting example, where the force is a centrifugal force, the force can be applied in a forward direction to concentrate the cells and functionalized nanoparticles, and then applied in the reverse direction to resuspend the cells and functionalized nanoparticles. The centrifugal force can also be applied and reversed two or more times. As one non-limiting example where the force is a electric force, the force can be applied by electrophoresis on a conductive or semiconductive surface, where the functionalized nanoparticles and cells are mixed by their different relative electrophoretic mobilities when under a potential bias (see, Su, H., et al., Electrophoresis, 23 1551-1557 (2002) and U.S. Patent Application Publication No. US 2003/0119028). The electrical force can also be applied and reversed two or more times.

In some embodiments, the imaging of the morphological features of the contacted cells can comprise measuring an optical property of the optical contrast agent. The optical property of the optical contrast agent can include or exclude, for example: absorbance, scattering, fluorescence, photoluminesence, Raman emission, and photoluminescent lifetime. The optical property of the optical contrast agent can be measured under a microscope with either a light field illumination or dark field illumination.

In some embodiments, the morphological features identified from the cell can include or exclude, for example: the shape of cellular features, for example, the cell surface shape, the cell nucleus shape, the chromatin shape, the nucleolar shape, the number of cellular features, such as the number of nucleoli or mitochondria, the density of staining of cellular features, or any combination of any of the foregoing, or any other imaged cellular feature or compartment.

In some embodiments, the method for detecting the biomarker-morphological profile of a cell can further comprise: (h) diagnosing the subject's condition based on the biomarker-morphological profile of each cell. In some aspects the subject's condition may include or exclude, for example, the presence of a hematological cancer, non-malignant hematological disorder, solid tumor, kidney disease, bladder disease, liver disease, or infectious disease. The hematological cancer can include or exclude leukemia, lymphoma, or multiple myeloma. The non-malignant hematological disorder can be anemia or sickle cell disease. The solid tumor can include or exclude breast cancer, lung cancer, prostate cancer, bone cancer, colorectal cancer, or bladder cancer. When the solid tumor is breast cancer, the biomarkers can include or exclude, for example, Her2 or Neu. In some embodiments, the kidney disease can include or exclude acute kidney injury, chronic kidney disease, lupus nephritis, kidney rejection, or preeclampsia. In some embodiments, the infectious disease can include or exclude, for example: HIV, hepatitis, sexually transmitted diseases, or sepsis. In some embodiments, the hematological cancer can further comprise circulating cancer cells.

In some embodiments, when the subject's condition is a cancer, the subject's condition can be further identified by the lineage of the malignancy, the stage, or state of remission. For example, the lineage of the malignancy can include or exclude, for example: negative, Myeloid line, Lymphoid T cell line, or Lymphoid B cell line.

In some aspects, the resonant light scattering from each observed complexed nanoparticle can be detected using evanescent or non-evanescent light. In some aspects, the non-evanescent light can be transmitted light. The resonant light scattering of the complexed nanoparticle can be detected when imaging under a dark field illumination. In some aspects, an illuminated slide holder can replace the darkfield condenser in the microscope. The illuminated slide holder can use total internal reflection to illuminate the slide holder. The illuminated slide holder can comprise optical fibers to deliver light to the edge to the slide.

In some embodiments, the detection of the resonant light scattering from each observed complexed nanoparticle can be completed in under, for example, 1 second, 500 milliseconds, or 200 milliseconds.

In some embodiments, the one or a plurality of functionalized nanoparticle species can be comprised from nanoparticles from 10 to 200 nm in diameter. In some embodiments, the nanoparticles can be comprised of, for example, Ag, Au, Pt, Pd, Rh, Ro, Al, Cu, Ru, Cr, Cd, Zn, Si, Se or mixtures or alloys thereof. The alloy can be an alloy of gold (Au) and silver (Ag). The alloy can be of copper (Cu) and Gold (Au). The nanoparticles can comprise mixtures of the listed metals in discrete shells or layers. When the nanoparticles comprise Si, the nanoparticles can have a Si shell, SiO₂ shell (silica) or Si core. In some embodiments, the nanoparticles can be spherical, tubular, cylindrical, pyramidal, cubic, egg-shaped, t-bone-shaped, urchin- or rose-like (with spiky uneven surfaces) or hollow shaped. In some embodiments, the nanoparticles may have a round, oval, triangular, square, egg-shaped, or a t-bone-shaped cross-section.

In some embodiments, the plurality of functionalized nanoparticle species can be from 2 to 50 different species of functionalized nanoparticle species. Each species of functionalized nanoparticle species can be functionalized with a different species of biomarker-binding moiety. For example, in some aspects of this disclosure, each species of functionalized nanoparticle species is functionalized with a different biomarker binding moiety. In some aspects of this disclosure, different biomarker binding moieties used in successive contact of the cell with different pluralities of functionalized nanoparticle species, may be bound to the same functionalized nanoparticle, as disclosed herein.

In some embodiments, when the nanoparticle species are functionalized with a biomarker binding moiety, e.g., an antibody or antibody fragment or other biomarker binding moiety that binds to one of the following: CD3, CD22, CD79a, Kappa, Lambda, Pax-5, ZAP-70, MPO, or TdT; the functionalized nanoparticle species can enter the cell and bind to its respective intracellular biomarker. The intracellular biomarker can be in a cellular region which can include or exclude, for example, the cytosol, nucleus, on the nuclear membrane, or in or on another cellular compartment or structure. In some embodiments, the functionalized nanoparticles are small enough to enter the cell without disrupting the cell membrane. The cells can be treated with a permeabilizer so as to allow the functionalized nanoparticles to enter the cell without disrupting the cell membrane. In some embodiments, the biomarker signature can be obtained by counting the number or proportion of each of the functionalized nanoparticle species per cell. The number of cells having identified normal or abnormal morphological profiles in the sample can be totaled, weighted, or otherwise determined.

In some embodiments, the step of (d) illuminating the nanoparticle-cell complexes with evanescent light and detecting the resonant light scattering from each observed complexed nanoparticle, to obtain a biomarker signature of each observed cell in the method of detecting the biomarker-morphological profile of a cell can further comprise:
(i) using a software program that counts the number of each of the functionalized nanoparticle species per cell and processes images in each cell in the field of view;
(ii) moving the field of view digitally;
(iii) using a software program to count the number of each of the functionalized nanoparticle species per imaged cell in the next field of view and repeating steps (ii) and
(iii) until the entire substrate area is analyzed;
(iv) digitally combining all images obtained to generate a single image covering the entire substrate area; and
(v) generating from the data obtained for the entire substrate area the number of each of the functionalized nanoparticle species per cell, the biomarker signature, of each substrate-adhered cell.

In some embodiments, the software program also stores the positional information for each observed and/or imaged cell.

In some embodiments, the field of view is from about 0.25 μm² to about 2.5 cm². In some embodiments, the field of view can be from about 100 μm² to about 1000 mm². In some embodiments, the field of view is a 5 microns by 5 microns. In some embodiments, the field of view is 100 mm by 100 mm. In some embodiments, the field of view is square-shaped. The sides of the square-shaped field of view can be from 0.25 microns up to 2.5 centimeters. The field of view can cover one cell, or a plurality of cells. In some embodiments, the field of view can cover the area of the entire slide.

In some embodiments, the step of (0 imaging morphological features of the contacted cells in the method of detecting the biomarker-morphological profile of a cell can further comprise:
(i) using a software program that processes images of morphological features of each cell in the field of view;
(ii) moving the field of view digitally;
(iii) using a software program to process images of morphological features of each cell in the next field of view and repeating steps (ii) and (iii) until the entire substrate area is analyzed;
(iv) digitally combining all images obtained to generate a single image covering the entire substrate area; and
(v) generating from the data obtained for the entire substrate area the morphological features of each substrate-adhered cell to detect the biomarker-morphological profile of each cell.

In some embodiments, a method for detecting the biomarker-morphological profile of a cell can comprise:
(a) providing a sample comprising cells from a subject;
(b) adhering the cells to a substrate;
(c) contacting the substrate-adhered cells with an optical contrast agent;
(d) imaging morphological features of the contacted cells;
(e) converting the optical contrast agent to a colorless form;
(f) contacting the cells with one or a plurality of functionalized nanoparticle species, each functionalized nanoparticle species comprising a biomarker-binding moiety to form nanoparticle-cell complexes;
(g) illuminating the nanoparticle-cell complexes with evanescent light and detecting the resonant light scattering from each imaged cell nanoparticle complex, to obtain a biomarker signature of each observed cell; and
(h) associating the morphological features of the contacted cells with the biomarker signature of each substrate-adhered cell to detect the biomarker-morphological profile of each cell.

In some embodiments, the optical contrast agent can be a leuco dye. The leuco dye can be methylene blue, methylene green, red leuco dye, crystal violet, phenolphthalein, or thymolphthalein. The leuco dye can be converted to a colorless form by the addition of one or more electrons to the dye. Electrons can be added to the dye via a reduction method. The reduction method can be effected by an electrochemical reduction, photoreduction, or reaction with a reducing agent. In some embodiments, the leuco dye can be converted to a colored form by the removal of one or more electrons from the dye. One or more electrons can be removed from the dye by an oxidation method. The oxidation method can be effected by an electrochemical oxidation, photooxidation, or reaction with an oxidation agent.

In some embodiments, the method for detecting the biomarker-morphological profile of a cell can further comprise: (d)(2) removing a first plurality of functionalized nanoparticles; and (d)(3) contacting the cells with a second plurality of functionalized nanoparticle species. In some embodiments, removing a first plurality of functionalized nanoparticles can be achieved by cleaving a linker between each species of functionalized nanoparticle and each species of a functionalized nanoparticle-associated biomarker-binding moiety. In some aspects of this disclosure, different biomarker binding moieties used in successive contact of the cell with different pluralities of functionalized nanoparticle species, may be bound to the same functionalized nanoparticle. For example, an anti-CD3 binding moiety may be bound to a 10 nm gold particle for use in contacting a first plurality of functionalized nanoparticle species with a cell, and an anti-CD8 antibody may be bound to a 10 nm gold particle for use in a second plurality of functionalized nanoparticle species, after the first plurality of functionalized nanoparticles has been released. In some aspects of this disclosure, different pluralities of functionalized nanoparticle species may detect biomarkers indicative of two or more diseases or conditions.

In some embodiments of this disclosure, the cells may be the same type or different types from each other. In some embodiments, the cells may be associated with different conditions. For example, cells may be associated with one or more of the following conditions: hematological cancer, non-malignant hematological disorder, solid tumor, bladder disease, liver disease, kidney disease, or infectious disease. In some aspects, the cells may be associated with two or more types of solid tumors.

In some embodiments, the removal of a first plurality of functionalized nanoparticles can be achieved by releasing the first plurality of functionalized nanoparticles from the biomarker binding moieties. The functionalized nanoparticles can be released by displacing, cleaving, separating, disconnecting, hydrolyzing, or dissociating the nanoparticles from the biomarker-binding moieties. In some embodiments, the linker between each nanoparticle species in the first plurality of nanoparticles and its respective biomarker binding moiety comprises a first oligonucleotide bound to a first nanoparticle species and a second oligonucleotide bound to its respective biomarker binding moiety, where the second oligonucleotide comprises a portion complementary to at least a portion of the first oligonucleotide, and hybridization of the first oligonucleotide to the second oligonucleotide forms a linker comprising a double-stranded nucleic acid in these oligonucleotide-linker functionalized nanoparticle species. In some embodiments the first, second and third oligonucleotides may be the same for each of the nanoparticle species and respective biomarker binding moiety in the first plurality of nanoparticles. Each nanoparticle species can be released from its respective biomarker binding moiety by binding of a third oligonucleotide to the first oligonucleotide with the hybrid formed by hybridization of the third oligonucleotide and the first oligonucleotide exhibiting a melting temperature higher than the melting temperature of the double-stranded nucleic acid formed by hybridization of the first and second oligonucleotide. In other embodiments, first, second and third oligonucleotides associated with each nanoparticle species and its respective biomarker binding moiety may be different for each nanoparticle species and its respective biomarker binding moiety. For example, in the first plurality of nanoparticles, the second nanoparticle species may comprise a fourth oligonucleotide, its respective biomarker binding moiety may comprise a fifth oligonucleotide, and the displacing oligonucleotide may be a sixth oligonucleotide.

In some aspects of this disclosure, each species of functionalized nanoparticle species can be functionalized with a different DNA oligonucleotide releasing system. In other aspects, all of the functionalized nanoparticle species in a plurality of functionalized nanoparticle species may be functionalized with the same DNA oligonucleotide releasing system. In some aspects all of the functionalized nanoparticle species used may be functionalized with the same or different DNA oligonucleotide releasing system.

In some embodiments, one or more iterations of interrogating cells and detecting biomarkers can be achieved by successive contacts with at least a second, third, up to ten or more plurality of nanoparticle species. In some embodiments, the one or more iterations of interrogating biomarkers can be one, two, three, four, five, six, seven, eight, nine, ten, fifteen, twenty, thirty, forty, fifty, sixty, seventy, or more times. In embodiments where oligonucleotide-linker functionalized nanoparticles are used, each plurality of nanoparticle species and respective biomarker binding moiety may comprise the same first, second third oligonucleotide for each oligonucleotide-linker functionalized nanoparticle species in a given plurality of oligonucleotide-linker functionalized nanoparticle species. Alternatively, each oligonucleotide-linker functionalized nanoparticle species and its respective biomarker binding moiety in each plurality of oligonucleotide-linker functionalized nanoparticle species may comprise a unique set of first, second and third oligonucleotides such that each biomarker binding moiety is associated with a unique set of first, second and third oligonucleotides. In this embodiment, from one, to ten or more successive rounds of displacement and contact with a new plurality of oligonucleotide-linker functionalized nanoparticle species can take place.

In some embodiments, after releasing the second or previous plurality of functionalized nanoparticles from the biomarker binding moieties, the following steps are performed:
(i) the biomarkers bound on or in the cell by the biomarker-binding moieties of the functionalized nanoparticles are classified, and
(ii) the cells are contacted with a next plurality of nanoparticles functionalized with different biomarker binding moieties, where each nanoparticle species of the next plurality of nanoparticles is functionalized with a biomarker binding moiety that binds to a biomarker suspected of being associated with samples or conditions, diseases, or disorders that are also associated with the first biomarker.
   In this aspect, the methods of this disclosure are useful in detecting whether the associated biomarkers are present on the same or different cell or populations of cells.

In some embodiments, the removing a first plurality of functionalized nanoparticles can be achieved by cleaving a linker between the nanoparticle and the biomarker-binding moiety. The linker can comprise a polynucleotide, modified polynucleotide, polyribonucleotide, modified polyribonucleotide, peptide, dextran or glycan. The polynucleotide can comprise a DNA restriction enzyme sequence. The modified polynucleotide can comprise a di-thiol, diol, abasic, or uracil moiety within the polynucleotide sequence.

In some embodiments, the linker can comprise a peptide that further comprises a protease sequence. The protease sequence can be a trypsin or chymotrypsin protease recognition sequence. In some embodiments, the linker can comprise a glycan that further comprises an alpha-fucosidase recognition site. The alpha-fucosidase recognition site can be an alpha-1,2 fucoside bond. In some aspects, the linker can be cleaved with a peptidase, DNAase, and/or RNAse.

In some embodiments, the substrate can be glass silica, clear polymer, gold, or alumina. The substrate can be functionalized. The substrate functionalization can be patterned. The substrate functionalization can be a silane-linked cell biomarker, polymer-linked cell biomarker, silane-linked amine, silane-linked carboxylic acid, polymer-linked amine, polymer-linked carboxylic acid, polyethylene glycol (PEG), gold, silver, alumina, dextran, or glass silica.

A combination or kit is described for the detection of a cellular biomarker signature, the combination comprising a plurality of biomarker-binding-moiety-functionalized nanoparticles.

A combination or kit is described for the detection of a cellular biomarker signature. In some embodiments, the combination can comprise a plurality of biomarker-binding moiety functionalized nanoparticles where the functionalized nanoparticles can comprise a mixture, or can be segregated. In some embodiments, the functionalized nanoparticles can further comprise: a nanoparticle functionalized with a first oligonucleotide; a biomarker-binding moiety functionalized with a second oligonucleotide, and the first oligonucleotide is complementary to a portion of the second oligonucleotide, and the first and second oligonucleotide form a hybridized duplex. In an alternative embodiment, the functionalized nanoparticles can further comprise a nanoparticle functionalized with a first oligonucleotide; a biomarker-binding moiety functionalized with a second oligonucleotide; and a third oligonucleotide, where the first oligonucleotide is complementary to a portion of the third oligonucleotide, the second oligonucleotide is complementary to a separate portion of the third oligonucleotide, and the first and second oligonucleotides form a hybridized duplex to the third oligonucleotide.

In some aspects, this disclosure relates to a kit for the detection of a biomarker signature, the kit comprising a combination comprising a plurality of functionalized nanoparticle species, each comprising a biomarker binding moiety. In some aspects, a kit may comprise a plurality of functionalized nanoparticles and an optical contrast agent. The optical contrast agent can be the optical contrast agents described herein.

In some aspects, the kit for the detection of a biomarker signature, comprising a combination comprising a plurality of functionalized nanoparticle species, may also comprise a mountant having substantially the same refractive index as the cells to be imaged. In some aspects, the mountant may have a refractive index within 0.1 of the refractive index of the cells to be imaged. The mountant may be any of the mountants described herein. As a non-limiting example, the mountant may have a refractive index of 1.52.

In some embodiments, the kit may comprises a plurality of biomarker-binding moiety functionalized nanoparticles where the nanoparticles can comprise mixture, or can be segregated. In some embodiments, for example, the functionalized nanoparticles can further comprise a functionalized nanoparticle, where the nanoparticle is releasable from the biomarker binding moiety. In some aspects, a first nanoparticle species is functionalized with a first oligonucleotide; a biomarker-binding moiety functionalized with a second oligonucleotide, and the first oligonucleotide is complementary to a portion of the second oligonucleotide, and the first and second oligonucleotide form a hybridized duplex. In an alternative embodiment, the functionalized nanoparticles can further comprise a nanoparticle functionalized with a first oligonucleotide; a biomarker-binding moiety functionalized with a second oligonucleotide; and a third oligonucleotide, where the first oligonucleotide is complementary to a portion of the third oligonucleotide, the second oligonucleotide is complementary to a separate portion of the third oligonucleotide, and the first and second oligonucleotides form a hybridized duplex to the third oligonucleotide.

A kit is described for the detection of a cellular biomarker signature. In some embodiments, the kit can comprise a plurality of functionalized nanoparticles, an optical contrast agent, and a mountant.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIG. 1 depicts the general process for the successive displacements of functionalized nanoparticles.

FIG. 2 depicts one embodiment for the displacement of a functionalized nanoparticle.

FIG. 3 depicts one embodiment for the displacement of a functionalized nanoparticle using a bridging oligonucleotide.

FIG. 4 depicts the process for the preparation of the functionalized nanoparticle with a biomarker-binding moiety. In the figure “b” denotes biotin, “SAv” denotes streptavidin.

FIGS. 5A and 5B: FIG. 5a depicts the process for the preparation of the functionalized nanoparticle with an oligonucleotide, and the preparation of a functionalized nanoparticle with a biomarker-binding moiety via a displaceable oligonucleotide overlap. FIG. 5a, depicts the formation of a functionalized nanoparticle by the coupling of an amine-functionalized oligonucleotide with a carboxylic acid-functionalized nanoparticle in the presence of EDC catalyst. FIG. 5a also depicts the formation of a functionalized nanoparticle by the coupling of streptavidin to a carboxylic acid-functionalized nanoparticle in the presence of EDC catalyst, followed by the subsequent coupling of a biotin-functionalized oligonucleotide to the streptavidin-coated nanoparticle. FIG. 5b depicts the formation of a functionalized nanoparticle by the reaction of a maleimide-functionalized oligonucleotide with a thiol (from a cysteine amino acid) on an antibody, followed by hybridization of the oligonucleotide-functionalized antibody to an oligonucleotide-functionalized nanoparticle containing a partial reverse complement sequence to the oligonucleotide sequence connected to the antibody.

FIG. 6 depicts the process for the preparation of the functionalized nanoparticle with an oligonucleotide, and the preparation of a functionalized nanoparticle with a biomarker-binding moiety via a displaceable bridging oligonucleotide.

FIG. 7 is a Brightfield Image of stained, functionalized nanoparticle labeled cells detection in Bright-Field using 20× objective, Olympus BX60M microscope and DP71 color camera.

FIGS. 8A and 8B: FIG. 8a is a Dark-field Image of stained, functionalized nanoparticle labeled cells at 20× objective on Olympus BX60M microscope in Dark-field utilizing the DarkLite Illuminator light source. FIG. 8b shows an expanded view of two of the selected stained, functionalized nanoparticle labelled cells from FIG. 8a.

FIG. 9 shows an initial Brightfield image of Giemsa stained blood smear stained cells imaged for morphology detection in Bright-Field using 20× objective, Olympus BX60M microscope and DP71 color camera.

FIG. 10 shows a Brightfield image of blood cells after destain treatment using 20× objective, Olympus BX60M microscope and DP71 color camera.

FIG. 11 shows a Dark-field image (100 ms exposure) of destained blood smear of the same field imaged for residual Giemsa stain (FIG. 4) using 20× objective on Olympus BX60M microscope in Dark-field utilizing DarkLite Illuminator light source.

FIG. 12 shows a Dark-field image (40× objective, 200 ms exposure, 400% zoom) of CEM cell labeled with three colors of nanoparticles.

FIG. 13 shows a Brightfield image (40× Objective, 0.1 ms exposure, 200% zoom) of CEM cell stained with Giemsa and labeled with 4 colors of nanoparticles.

FIG. 14 shows a Dark-field image (40× Objective, 100 ms exposure, 200% zoom) of CEM cell labeled with 4 colors of nanoparticles.

FIG. 15 shows a Dark-field image (40× Objective, 100 ms exposure) of cells contacted with functionalized nanoparticles in the absence of a RI-matched mountant.

FIG. 16 shows Dark-field image of a cell sample where the cells were labeled without applying additional force (Passive Labeling).

FIG. 17 shows a Dark-field image of a cell sample where the cells were labeled with functionalized nanoparticles using centrifugation (additional gravitational force).

FIG. 18 shows a combined phase contrast and Dark-field image of a blood smear where cells were labeled using centrifugation (additional gravitational force).

FIGS. 19A-J shows a Brightfield image (FIG. 19A) of a blood smear where cells were labeled using multiplex labeling and Giemsa staining. FIG. 19B shows a darkfield image of the same blood smear with the same field of view where cells are labelled using multiplex labeling and Giemsa staining. FIG. 19D and FIG. 19C show expanded views of selected labelled cells which were also Giemsa stained and observed at the same location in the Brightfield image. Au anti-CD-3 (yellow/lighter) and Ag anti-CD4 (blue/darker) functionalized nanoparticles bind to the four lymphocytes in the field. No functionalized nanoparticle binding to neutrophils was detected (FIG. 19F), whereas the neutrophils were observed in the Brightfield image with a Giemsa stain (FIG. 19E). Au anti-CD-3 (yellow/lighter) and Ag anti-CD4 (blue/darker) functionalized nanoparticles bound to the four lymphocytes in the field (FIG. 19H and FIG. 19J). FIG. 19G and FIG. 19I show the corresponding Brightfield image of the Giemsa-stained lymphocytes as those observed in FIG. 19H and FIG. 19J, respectively.

FIGS. 20A-D shows a Brightfield image of a whole blood cell suspension where cells were labeled with Au anti-CD3. FIG. 20A shows the Darkfield image, and FIG. 20B shows the corresponding Brightfield image of the same blood smear with the same field of view. Au anti-CD3 (yellow/lighter colors) functionalized nanoparticles bound to 13 out of 14 lymphocytes in the field, as observed by comparing the labelled cells in FIG. 20A with the Giemsa-stained cells in FIG. 20B. FIG. 20C and FIG. 20D show an expanded view of lymphocytes labelled with functionalized anti-CD3 Au nanoparticles. No functionalized nanoparticle binding to neutrophils was detected.

FIGS. 21A and 21B show a passively incubated slide (A) and a slide electronically enhanced in functionalized nanoparticle density (B).

FIG. 22 shows 50 nm (Green/Darker) and 70 nm (Yellow/Brighter) Au Nanoparticles on FFPE Tissue.

### DETAILED DESCRIPTION

The presently disclosed subject matter is described more fully hereinafter with reference to the accompanying description and drawings, in which some, but not all embodiments of the presently disclosed subject matter are shown. The presently disclosed subject matter can be embodied in many different forms and should not be construed as limited to the embodiments set forth herein; instead, these embodiments are provided so that this disclosure will satisfy applicable legal requirements. Like numbers refer to like elements throughout.

Many modifications and other embodiments of the presently disclosed subject matter set forth herein will be understood by one skilled in the art to which the presently disclosed subject matter pertains having the benefit of the teachings presented in the foregoing descriptions and the associated drawings. Therefore, the presently disclosed subject matter is not to be limited to the specific embodiments disclosed, and those of skill in the art will appreciate that modifications and other embodiments are included within the scope of the appended claims. Although specific terms are employed herein, they are used in a generic and descriptive sense only and not for purposes of limitation. The disclosure utilizes the abbreviations shown below.

### Abbreviations

DNA—deoxyribonucleic acid
RNA—ribonucleic acid
TIRF—total internal reflection fluorescence
PEG—polyethylene glycol

### CD—Cluster Determination

ScFv—single chain variable fragment
DPX—dibutyl phthalate-xylene mountant
RI—refractive index
NADH—Nicotinamide adenine dinucleotide, reduced
NAD+—Nicotinamide adenine dinucleotide, oxidized
NADP+—nicotinamide adenine dinucleotide phosphate
DTT—dithioerythritol
EDC—1-Ethyl-3-(3-dimethylaminopropyl)carbodiimide
DMSO—dimethylsulfoxide

Unless specific definitions are provided, the nomenclature utilized in connection with, and the laboratory procedures, techniques and methods described herein are those known in the art to which they pertain. Standard chemical symbols and abbreviations are used interchangeably with the full names represented by such symbols. Thus, for example, the terms “hydrogen” and “H” are understood to have identical meaning. Standard techniques may be used for chemical syntheses, chemical analyses, pharmaceutical preparation, formulation, delivery, and treatment of patients. Standard techniques may be used for recombinant DNA methodology, oligonucleotide synthesis, tissue culture and the like. Reactions and purification techniques may be performed e.g., using kits according to manufacturer's specifications, as commonly accomplished in the art or as described herein.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety. Moreover, any listing of multiple values are understood to include any range between any two of the listed values.

The terms “a”, “an”, and “the” refer to “one or more” when used in this application, including the claims. Thus, for example, reference to “a cell” includes a plurality of such cells, unless the context clearly is to the contrary (e.g., a plurality of cells), and so forth.

If not otherwise specified, and where applicable, the term “substantially” when used in association with a numerical term may refer to 85, 86, 87, 88, 89, 90, 91, 92, 93, 94, 95, 96, 97, 98, 99, or 99.5%. For example, the term “substantially” reduced may refer to 85% or greater reduction in the recited property. The term “biomarker signature” or “biomarker profile” are not numerical terms for purposes of this definition.

### Detecting Cell Biomarker Signatures and Biomarker-Morphological Profiles Using Resonance-Light Scattering

In some embodiments, the present disclosure features methods and compositions for detecting cell-nanoparticle binding moiety complexes useful in detecting a biomarker signature of a cell. The methods and compositions are also useful, in some embodiments, for detecting the biomarker-morphological profile of an imaged cell.

In some embodiments, the nanoparticles functionalized with biomarker-binding moieties can be used for detecting functionalized nanoparticle cell complexes, which are useful, for example, for identifying and quantifying biomarkers present on cells. In some embodiments, the cells may also be imaged to detect morphological features of the cells complexed with functionalized nanoparticles. In some embodiments, the functionalized nanoparticles can be contacted with the same cells analyzed by a morphological imaging analysis to obtain a biomarker-morphological profile that cell.

In some embodiments, the methods of this invention are useful for improving signal generation, detection limits, dynamic range, and/or performance characteristics of the biomarker signature assays. For example, in some embodiments, the present disclosure relates to methods for increasing the loading amount of a biomarker binding moiety onto a cell by using an external force to increase the local concentration of the functionalized nanoparticles and cells. In some embodiments, the biomarker binding moiety may be a functionalized nanoparticle species. In some aspects, the external force may be a centrifugal, or magnetic force. In some aspects of this invention, the method of detecting functionalized nanoparticle cell complexes can comprise:
(a) providing a sample comprising cells from a subject;
(b) contacting the cells with one or a plurality of functionalized nanoparticle species, each functionalized nanoparticle species comprising a biomarker-binding moiety, wherein an external force is used to accelerate the formation of nanoparticle-cell complexes through binding of the nanoparticle species comprising a biomarker-binding moiety to its respective biomarker;
(c) adhering the functionalized nanoparticle-cell complexes to a substrate;
(d) detecting the functionalized nanoparticle cell complexes by illuminating the nanoparticle-cell complexes with evanescent light and detecting the resonant light scattering from each observed functionalized nanoparticle cell complex, to obtain a biomarker signature of each observed cell; and
(e) associating the biomarker signature of each substrate-adhered cell to a known disease, condition, or state of a cell exhibiting substantially the same biomarker signature to identify the disease, condition, or state of the cell from a subject.

In some aspects, the phrase substantially the same or similar biomarker signature, or biomarker-morphological profile, may refer to a biomarker signature or profile comprising similar levels and/or types of biomarkers and/or biomarker-morphologies for which a concordance has been established or reasonably is expected for the same disease, condition, state or disorder.

In some embodiments, the biomarker signature of a cell can be detected in a homogeneous assay, the assay comprising the steps of:
(a) providing a sample comprising cells from a subject;
(b) contacting the cells with one or a plurality of functionalized nanoparticle species, each functionalized nanoparticle species comprising a biomarker-binding moiety, and forming functionalized nanoparticle-cell complexes through binding of the nanoparticle species comprising a biomarker-binding moiety to its respective biomarker;
(c) adhering the functionalized nanoparticle-cell complexes to a substrate;
(d) illuminating the functionalized nanoparticle-cell complexes with evanescent light and detecting the resonant light scattering from each observed complexed nanoparticle, to obtain a biomarker signature of each observed cell,
   where unbound functionalized nanoparticles are not removed from the field of view.

Often, unbound species are washed from a target to reduce background noise. In some embodiments wash steps are omitted, leaving unbound functionalized nanoparticles in the field of view. This embodiment may be used in some embodiments, for example, for high throughput assays, and/or automated assays. The functionalized nanoparticles are specific to the biomarker on the cell, and can substantially contact the cell such that litle to none signal is observed for the unbound functionalized nanoparticles.

In some embodiments, the disease, condition, or state of a cell can be identified by forming and detecting complexes between the functionalized nanoparticles and cells. The method can comprise associating a biomarker signature of each substrate-adhered cell to a known disease, condition, or state of a cell exhibiting substantially the same biomarker signature to identify the disease, condition, or state of the cell from a subject.

In some embodiments of this invention, where biomarker signatures are detected, the detecting of the resonant light scattering from each observed complexed nanoparticle comprises imaging the cell-functionalized nanoparticle complexes in contact with a mountant. In some embodiments, background and/or interfering white light scatter can be reduced or substantially eliminated by using a mountant comprising a solution with about the refractive index of the cells. In some embodiments of this invention, the mountant can be within about 0.1 of the refractive index of cells, where the cells are fixed. In some embodiments, the refractive index of fixed cells is about 1.52, or 1.52. In some embodiments, the index of refraction of the mountant can be from 1.51 to 1.54. In some embodiments, using a mountant having an RI within 0.1 of the refractive index of fixed cells is useful for reducing the amount of white light scatter, and obtaining better images of resonance scattering from the cell-functionalized nanoparticle complexes. In one aspect, this disclosure relates to a method for detecting functionalized nanoparticle cell complexes, the method comprising:
(a) providing a sample comprising cells from a subject;
(b) contacting cells which have been fixed with one or a plurality of functionalized nanoparticle species, each functionalized nanoparticle species comprising a biomarker-binding moiety, and forming nanoparticle-cell complexes through binding of the nanoparticle species comprising a biomarker-binding moiety to its respective biomarker;
(c) adhering the functionalized nanoparticle-cell complexes to a substrate, wherein the adhered nanoparticle-cell complexes are placed in contact with a mountant, wherein the refractive index of the mountant is within about 0.1 of the refractice index of the fixed cells;
(d) detecting the functionalized nanoparticle cell complexes by illuminating the nanoparticle-cell complexes with evanescent light and detecting the resonant light scattering from each observed functionalized nanoparticle cell complex, to obtain a biomarker signature of each observed cell; and
(e) associating the biomarker signature of each substrate-adhered cell to a known disease, condition, or state of a cell exhibiting substantially the same biomarker signature to identify the disease, condition, or state of the cell from a subject.
   In some embodiments, the mountant may have a refractive index of about 1.52.

In some embodiments, the ability to use the compositions and methods of this disclosure to associate the biomarker signature of an individual cell, and in some embodiments, with its morphological image/features greatly enhances the ability to diagnose and monitor abnormal conditions or disorders.

As set forth herein, the inventors have made the surprising discovery that nanoparticles functionalized with biomarker-binding moieties can be used for detecting cell-functionalized nanoparticle complexes and identifying and quantifying biomarkers present on imaged cells for example, when the functionalized nanoparticles are contacted with the same cells analyzed by a morphological imaging analysis. The ability to use the compositions and methods of this disclosure to associate the biomarker signature of an individual cell with its morphological image/features greatly enhances the ability to diagnose and monitor abnormal conditions or disorders.

In some embodiments, this disclosure relates to compositions and methods for obtaining a biomarker signature for an imaged cell, which is used, in some embodiments, in combination with detected morphological features of the cell obtained from imaging the cell. In some aspects, compositions comprising functionalized nanoparticle species, each comprising a specific biomarker-binding moiety, are used to detect the biomarker signature of the imaged cell. In some embodiments of this disclosure, combinations of such compositions are made or used in the methods of this disclosure. The combinations can be mixtures of such compositions, or may comprise compositions segregated before use.

In some embodiments, the method for detecting a biomarker-morphological profile of a cell can comprise the steps of
(a) providing a sample comprising cells from a subject;
(b) contacting the cells with one or a plurality of functionalized nanoparticle species, each functionalized nanoparticle species comprising a biomarker-binding moiety, and forming nanoparticle-cell complexes through binding of the nanoparticle species comprising a biomarker-binding moiety to its respective biomarker on the cell;
(c) adhering the functionalized nanoparticle-cell complexes to a substrate;
(d) illuminating the functionalized nanoparticle-cell complexes with evanescent light and detecting the resonant light scattering from each observed complexed nanoparticle, to obtain a biomarker signature of each observed cell;
(e) contacting the substrate-adhered cells with an optical contrast agent;
(f) imaging morphological features of the contacted cells; and
(g) associating the morphological features of the contacted cells with the biomarker signature of each substrate-adhered cell to detect the biomarker-morphological profile of each cell.

In addition, it may be desirable to detect the normal or diseased tissue or cells of a patient. The presence or absence of certain circulating cancer or other cells, for example, may be diagnostic for disease. Thus, the endogenous cells of a human patient are the cells that may be advantageously detected using the compositions, methods and kits of the present invention.

### Sample Source

The term “sample” as used herein refers to an aliquot of material, frequently an aqueous solution or an aqueous suspension derived from biological material. In some embodiments, the sample can be a biological sample. The biological sample can be from a living subject. For example, in some embodiments, the sample may be any sample containing cells. In some embodiments, the sample may be from, for example, whole blood, bone marrow, serum, plasma, cerebrospinal fluid, sputum, bronchial washings, bronchial aspirates, urine, lymph fluids and various external secretions of the respiratory, intestinal and genitourinary tracts, tears, saliva, milk, white blood cells, myelomas and the like; biological fluids such as cell culture supernatants, tissue specimens which may or may not be fixed, and cell specimens which may or may not be fixed, or a fine needle aspirate. Samples to be assayed for the presence of an analyte by the methods of the present invention include, for example, cells, tissues, homogenates, lysates, extracts, purified or partially purified proteins and other biological molecules and mixtures thereof. As a non-limiting example, the tissue sample can be a tissue sample from a biopsy, for example, a FFPE (formalin-fixed, paraffin-embedded) tissue sample, aspirate, or surgically removed tissue sample. The FFPE samples can be sourced from a clinic or laboratory. The samples used in the methods of the present invention will vary based on the assay format and the nature of the tissues, cells, extracts or other materials, especially biological materials, to be assayed.

In some embodiments, the biological sample may be processed. The processing can be, for example, removal of selected species in the sample. In some embodiments, the sample may comprise white blood cells. In some embodiments, at least 10%, 20%, 30%, 40%, 50%, 60%, 70%, 80%, or 90% of the red blood cells are removed before contacting the cells with the plurality of functionalized nanoparticle species. In some embodiments, at least 50% of the red blood cells are removed before contacting the cells with the plurality of functionalized nanoparticle species.

As used herein, “subject” refers to any mammal that can include or exclude humans, domestic and farm animals, and zoo, or pet animals, such as dogs, horses, cats, mouse, rat, llama, sheep, pigs, cows, etc. The preferred mammal herein is a human, including adults, children, and the elderly. Preferred sports animals are horses and dogs. Preferred pet animals are dogs and cats. The subject may be, for example, an aquatic park animal, such as a dolphin, whale, seal or walrus. In certain embodiments, the subject, individual or patient is a human. Cells for use in the present invention may be obtained from any of the aforementioned subjects.

In some embodiments, cells from a subject can comprise biomarkers which may be useful to assist in identifying the cellular state, identity, growth rate, lineage, mutations, variants, expression levels, cancer stage or remission status, and/or latent or active infection. Such cells may include or exclude, for example, mammalian cells, immunomodulatory cells, lymphocytes, monocytes, polymorphs, T cells, tumor cells, yeast cells, bacterial cell, infectious agents, parasites, plant cells, transfected cells such as NSO, CHO, COS, 293 cells.

In some embodiments, the cell may be alive, dead, fixed and/or substantially intact. In some embodiments, the cells can be the same type or different types. When the cells are different types, the cells can be from different tissue or tumor origin, exhibit a different pathology, express different or mutated biomarkers, express different levels of biomarkers, express biomarkers with different post-translational modifications, or exhibit different morphology. In some embodiments, the biomarker-binding moiety is capable of distinguishing between a mutant biomarker and a wild-type biomarker. When the cells are from different tumor origin, the cells can be from a tumor which can include or exclude, for example, breast cancer, lung cancer, prostate cancer, bone cancer, colorectal cancer, liver cancer, pancreatic cancer, thyroid cancer, bladder cancer, or other types of cancer.

The terms “cell proliferative disorder” and “proliferative disorder”, as used herein, refer to disorders that are associated with some degree of abnormal cell proliferation. In some embodiments, the cell proliferative disorder is cancer.

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer” and “cancerous,” as used herein, refer to or describe the physiological condition in mammals that is typically characterized by unregulated cell growth and/or proliferation. Some cancers are composed of rapidly dividing cells while others are composed of cells that divide more slowly than normal. Types of cancer examples can include or exclude, for example, carcinoma, lymphoma (e.g., Hodgkin's and non-Hodgkin's lymphoma), blastoma, sarcoma, and leukemia. More particular examples of such cancers can include or exclude, for example, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

In some embodiments, local cancer metastases can invade the lymphatic system, leading to distant mestastases. Distant metastases commonly involve the brain, lung, bone and liver. Each cancer has a distinct pattern of metastasis (e.g. prostate cancer may metastasizes to the bone, but rarely to the brain). Metastasis can occur at any time in cancer growth, and may occur before or after removal of the primary tumor. A metastasized cancer cell can retain many of the characteristics of the original cancer cells. For example, in some embodiments, the methods of this disclosure can detect the origin of metastasizing cancer. The origin of metastasizing cancer can be ascertained by identifying the biomarker signature and/or biomarker-morphological profile of tumor cells in a distant location, and detecting that the biomarker signature and/or biomarker-morphological profile is the same or similar to the biomarker signature and/or biomarker-morphological profile from the primary cancer at its original location. In some embodiments, a cancer cell in a tissue may exhibit multiple neoplasms. In some embodiments, the tissue cells can be interrogated separately, such that one or more cancer cell types (or neoplasm) can be identified during the detecting a biomarker or biomarker-morphological profile of a cell.

### Histopathology Methods

Histopathology imaging often requires cell immobility such that the same region can be analyzed after subjecting the cells in the region with a different imaging modality to correlate the cells from one imaging modality to the other imaging modality. In some embodiments, the cell is fixed with a fixing agent. The fixing agent may be, for example, formaldehyde, glutaraldehyde, or another cross-linking agent. In other embodiments water-soluble preservatives, for example, methyl or propyl paraben, dimethylolurea, sorbic acid, 2-pyridinethiol-1-oxide, or potassium sorbate may be used. In some embodiments the cell is permeabilized by surfactants.

In some embodiments, the functionalized nanoparticle cell complexes adhered to a substrate may be imaged in contact with a mountant. As used herein, a “mountant” is any substance in which a specimen is suspended between a slide and a cover glass for microscopic examination. A mountant can be used to maintain image fidelity during the course of the detection. One of the major causes of image degradation in microscopy is due to improper matching of the refractive index between the immersion medium and mountant (Diaspro A, et al., Appl Opt 2002; 41(4):685-690). However, if the mountant refractive index is different from the functionalized nanoparticle cell complex, white light scattering will result.

In some embodiments, the mountant can comprise a solution with a similar refractive index to the refractive index of the cells. The refractive index of a cell may vary by cell type, and may also vary within the region of the cell. In some aspects, the refractive index of cells may vary from 1.2 to 1.6. In some aspects, the refractive index may vary from 1.4 to 1.5. In some embodiments, the refractive index of fixed cells can be 1.52. In some embodiments, the mountant can be within 0.1 of the refractive index of the fixed cells. In some embodiments, the mountant can be within 0.09, 0.08, 0.07, 0.06, 0.05, 0.04, 0.03, 0.02, or 0.01 of the refractive index of the fixed cells. In some embodiments, the mountant can be within 1, 2, 3, 4, 5, 6, 7, 8 9, or 10% of the refractive index of the reference index of the fixed cells. In some embodiments, the mountant can be immersion oil, DPX, dissolved polystyrene (in xylene), Valap (an equal mixture of Vaseline, lanolin, and paraffin mixed on a heating plate 60° C.), Gel/Mount, Fluoromount-G, Fluorsave, Prolong, Vectashield, MOWIOL, Modified Apathy's mountant, Permount, or Entellan. The volume of the mountant can be from about 2 microliters to about 40 microliters. In some embodiments, the volume of mountant can be from 5 microliters to 15 microliters. In some embodiments, the volume of mountant can be about 10 microliters. In some embodiments, the mountant may further comprise an antifade reagent which prevents photodegradation of the optical contrast agent. In some embodiments, the mountant can further comprise spacers or gaskets. In some aspects, the spacers or gaskets can be tailored to direct the fluid flow to the cells. In some embodiments, algorithms can may be used to minimize the light noise from the white light scattering.

### Biomarker-Binding Moieties

The term “biomarker” as used herein refers to any distinguishing element found on or within a cell. The distinguishing element can be an antigen or another binding partner recognized by a biomarker binding moiety. The term “antigen” or “binding partner” as used herein refers to any known or unknown substance that can be recognized by an antibody or other biomarker binding moiety. The term “antigen” or “binding partner” may include, for example, proteins, peptides, glycoproteins and carbohydrates. In some embodiments, the antigen is expressed on the surface of a cell. Preferably these antigens include biologically active proteins, such as hormones, cytokines, and their cell surface receptors, or bacterial or parasitic cells, agents or antigens, membranes or purified components thereof, and viral antigens or binding partner. Such cells or agents may be those that naturally express the antigen or binding partner on their surface or a transformed cell expressing the antigen on its surface. In some embodiments, the transformed cell can be transfected with an oncogene which is integrated into the cell. In some embodiments, the transformed cells may include or exclude, for example, mammalian cells, immunomodulatory cells, lymphocytes, monocytes, polymorphs, T cells, tumor cells, yeast cells, bacterial cell, infectious agents, parasites, plant cells, transfected cells such as NSO, CHO, COS, 293 cells. Transformation of cells such as NSO, CHO, COS and 293 cells can be achieved by a method which can include or exclude electroporation and nucleofection. In some embodiments, the detected biomarker can be present on the cell surface, within the cell, or both on the surface and within the cell. In some embodiments the biomarker in the cell may be present in or on one or more cellular features, for example, the cytosol, the nucleus, the nuclear membrane, nucleoli, the endoplasmic reticulum, Golgi apparatus or mitochondria. In some embodiments, the biomarker can be expressed and transported to the cell surface which is accessible to external biomarker-binding moieties.

The terms “specifically binding” and “specific binding” as used herein mean that an antibody or other molecule, especially a biomarker-binding moiety of the invention, binds to a target such as an antigen, ligand or other analyte, with greater affinity than it binds to other molecules under the specified conditions of the present invention. In various embodiments of the invention, “specifically binding” may mean that an antibody or other specificity molecule binds to a target analyte molecule with at least about a 10⁶-fold greater affinity, preferably at least about a 10⁷-fold greater affinity, more preferably at least about a 10⁸-fold greater affinity, and most preferably at least about a 10⁹-fold greater affinity than it binds molecules unrelated to the target molecule. Typically, specific binding refers to affinities in the range of about 10⁶-fold to about 10⁹-fold greater than non-specific binding. In some embodiments, specific binding may be characterized by affinities greater than 10⁹-fold over non-specific binding. Whenever a range appears herein, as in “1-10 or one to ten, the range refers without limitation to each integer or unit of measure in the given range. Thus, by 1-10 it is meant each of 1, 2, 3, 4, 5, 6, 7, 8, 9, 10 and any subunit in between.

In some embodiments, the biomarker can be a biomolecule identified by the Cluster Determinant antigen (CD) and or other molecules/antigenic sites.

In some embodiments, the biomarker can include or exclude, for example, those listed in Table 1.

TABLE 1

| Antigen Name | Other Names |
|---|---|
| CD1a | R4, T6 |
| CD1b | R1, T6 |
| CD1c | M241, R7, T6 |
| CD1d | R3G1 |
| CD1e | cR2 |
| CD2 | T11, LFA-2, SRBC-R |
| CD2R | T11-3 |
| CD3 gamma, | T3g |
| CD3 delta | |
| CD3 epsilon | T3e |
| CD4 | L3T4, W3/25, T4 |
| CD5 | T1, Tp67, Leu-1, Ly-1 |
| CD6 | T12. TP120 |
| CD7 | gp40, TP41 |
| CD8a | T8, Leu-2 |
| CD8b | CD8, Leu2, Lyt3 |
| CD9 | p24, MRP-1 |
| CD10 | CALLA, NEP, gp100 |
| CD11a | LFA-1, integrin alphaL |
| CD11b | Mac-1, integrin alphaM |
| CD11c | p150, 95, CR4, integrin alphaX |
| CD12 | p90-120 |
| CD13 | Aminopeptidase N, APN |
| CD14 | LPS-R |
| CD15 | Lewis-x, Lex |
| CD15s | Sialyl Lewis X |
| CD15u | Sulfated Lewis X |
| CD16a | FcgammaRIIIA |
| CD16b | FcgammaRIIIB |
| CD17 | Lactosylceramide, LacCer |
| CD18 | Integrin beta2 |
| CD19 | B4 |
| CD20 | B1, Bp35 |
| CD21 | C3DR, CR2, EBV-R |
| CD22 | BL-CAM, Siglec-2 |
| CD23 | FcepsilonRII |
| CD24 | BA-1 |
| CD25 | Tac, p55 |
| CD26 | DPP IV |
| CD27 | T14 |
| CD28 | Tp44, T44 |
| CD29 | Integrin betal |
| CD31 | PECAM-1 |
| CD32 | FcgammaRII |
| CD33 | p67, Siglec-3 |
| CD34 | gp105-120, Hematopoietic progenitor cell antigen 1 |
| | (HPCA1) |
| CD35 | CR1 |
| CD36 | GPIV |
| CD37 | gp52-40, Leukocyte antigen CD37, Tetraspanin-26, |
| | TSPAN26 |
| CD38 | T10 |
| CD39 | Ectonucleoside triphosphate diphosphohydrolase 1 |
| | (ENTPD1), ATPdehydrogenase, |
| | NTPdehydrogenase-1 |
| CD40 | Bp50, MGC9013, TNFRSF5, Tumor necrosis |
| | factor receptor superfamily member 5 |
| CD41 | gpIIb |
| CD42a | GPIX |
| CD42b | GPIba |
| CD42c | GPIbb |
| CD42d | GPV |
| CD43 | Leukosialin, sialophorin |
| CD44 | H-CAM, Pgp-1 |
| CD44R | CD44v |
| CD45 | LCA, T200, B220 |
| CD45RA | PTPRC |
| CD45RB | PTPRC |
| CD45RO | |
| CD46 | Membrane Cofactor Protein (MCP), Trophoblast |
| | leukocyte common antigen, TRA2.10 |
| CD47R | |
| CD48 | Blast-1 |
| CD49a | VLA-1 |
| CD49b | VLA-2 |
| CD49c | VLA-3 |
| CD49d | VLA-4 |
| CD49f | VLA-6 |
| CD50 | ICAM-3 |
| CD51 | Vitronectin receptor |
| CD52 | CAMPATH-1 |
| CD53 | MOX44, TSPAN25, Tetraspanin-25 |
| CD54 | ICAM-1 |
| CD55 | DAF |
| CD56 | NCAM |
| CD57 | FINK-1, Leu-7 |
| CD58 | LFA-3 |
| CD59 | Protectin, MAC- inhibitor |
| CD60a | GD3 |
| CD60b | 9-O-sialyl GD3 |
| CD60c | 7-O-sialyl GD3 |
| CD61 | GPIIIa, Integrin beta-3 |
| CD62E | E-selectin, ELAM-1 |
| CD62L | L-selectin, LECAM-1 |
| CD62P | P-selectin, PADGEM, GMP-140 |
| CD63 | LIMP, MLA1, gp55, NGA, LAMP-3, ME491, |
| | OMA81H, TSPAN30, Granulophysin, |
| | Melanoma 1 antigen |
| CD64 | FcgRI, Fc-g Receptor 1, High affinity |
| | immunoglobulin g Fc Receptor I, FcgRIA |
| CD65 | Ceramide-dodecasaccharide, VIM2, |
| | Fucoganglioside (Type II) |
| CD65s | VIM2 |
| CD66a | BGP-1, NCA-160 |
| CD66b | CD67, CGM6 |
| CD66c | NCA |
| CD66d | CGM1 |
| CD66e | CEA |
| CD66f | PSG, Sp-1 |
| CD68 | Macrosialin, gp110 |
| CD69 | AIM, EA 1, MLR3, gp34/28, VEA, CLEC2C, |
| | BL-AP26 |
| CD70 | TNFSF7, CD27LG, CD27L, Ki-24 |
| CD71 | TFRC, T9, Transferrin receptor, TFR, TRFR |
| CD72 | Ly-19.2, Ly-32.2, Lyb2 |
| CD73 | Ecto-5′-nuclotidase, NT5E, E5NT, NT5, NTE, |
| | eN, eNT |
| CD74 | DHLAG, HLADG, Ia-g, li, invariant chain |
| CD75 | ST6GAL1, MGC48859, SIAT1, ST6GALL, ST6N, |
| | ST6 b-Galactosamide a-2,6-sialyltranferase, |
| | Sialo-masked lactosamine, Carbohydrate of a2,6 |
| | sialyltransferase |
| CD75s | a2,6 Sialylated lactosamine |
| CD77 | Gb3, Pk blood group |
| CD79a | IGA (Immunoglobulin-associated a), MB-1 |
| CD79b | IGB (Immunoglobulin-associated b), B29 |
| CD80 | CD28LG, CD28LG1, L AB7, B7, B7-1, BB1 |
| CD81 | TAPA-1 |
| CD82 | 4F9, C33, IA4, KAI1, R2, ST6, SAR2, GR15 |
| CD83 | HB15, BL11 |
| CDw84 | LY9B, SLAMF5, p75, GR6, hly9-b |
| CD85a | ILT5, LIR3, HL9, LILRB3 (Leukocyte |
| | immunoglobulin-like receptor, subfamily B (with |
| | TM and ITIM domains), member 3, LIR-3, |
| | MGC138403, PIRB, XXbac-BCX105G6.7 H |
| CD85c | LILRB5 (Leukocyte immunoglobulin-like receptor, |
| | subfamily B)(with TM and ITIM |
| | domains), member 5, LIR8 |
| CD85d | LILRB2 (Leukocyte immunoglobulin-like receptor, |
| | subfamily B (with TM and ITIM |
| | domains), member 2, LILRB2, ILT4, LIR2, MIR10, |
| | MIR-10 |
| CD85e | LILRA3 (Leukocyte immunoglobulin-like |
| | receptor, subfamily A (without TM domain), |
| | member 3, HM31, HM43, ILT6, LIR-4, |
| | LIR4, e3 |
| CD85f | XXbac-BCX403H19.2, CD85, CD85F, LIR9, ILT11, |
| | LILRB7 (Leukocyte immunoglobulin-like |
| | receptor, subfamily B (with TM and ITIM domains), |
| | member 7 |
| CD85g | LILRA4 (Leukocyte immunoglobulin-like receptor, |
| | subfamily A (with TM domain), |
| | member 4, ILT7, MGC129597, MGC129598 |
| CD85h | LILRA2 (Leukocyte immunoglobulin-like receptor, |
| | subfamily A (with TM domain), |
| | member 2, ILT1, LIR7, LIR-7, XXbac-BCX85G21.2, |
| | ILT-1 |
| CD85i | LILRA1 (Leukocyte immunoglobulin-like receptor), |
| | subfamily A (with TM domain), |
| | member 1, LIR-6, LIR6, MGC126563 |
| CD85j | LILRB1 (Leukocyte immunoglobulin-like receptor, |
| | subfamily B (with TM and ITIM |
| | domains), member 1, FLJ37515, ILT2, LIR-1, LIR1, |
| | MIR-7, MIR7 |
| CD85k | LILRB4 (Leukocyte immunoglobulin-like receptor, |
| | subfamily B (with TM and ITIM |
| | domains), member 4, ILT3, LIR-5, HM18, LIR5, |
| | LILRB5 |
| CD86 | B70, B7-2 |
| CD87 | UPA-R |
| CD88 | C5aR |
| CD89 | FcalphaR |
| CD90 | Thy-1 |
| CD91 | LRP1, a2M-R, a2MR, APOER, APR, LRP |
| CD92 | SLC44A1, CTL1, CHTL1, RP11-287A8.1, p70 |
| CD93 | C1QR1, C1qRP, MXRA4, C1qR(P), Dj737e23.1, |
| | GR11 |
| CD94 | KP43 |
| CD95 | CD178, FASLG, APO-1, FAS, TNFRSF6, CD95L, |
| | APT1LG1, APT1, FAS1, FASTM, |
| | ALPS1A, TNFSF6, FASL |
| CD96 | TACTILE, MGC22596 |
| CD97 | TM&LN1, BL-KDD/F12 |
| CD98 | SLC3A2, 4F2, 4F2HC, 4T2HC, MDU1, NACAE, |
| | FRP-1, RL-388 |
| CD99 | MIC2, E2 |
| CD99R | E2 |
| CD100 | SEMAJ, coll-4, C9orf164, FLJ33485, FLJ34282, |
| | FLJ39737, FLJ46484, M-sema-G, |
| | MGC169138, MGC169141, SEMA4D, SEMAJ |
| CD101 | IGSF2, P126, V7, BA27, BPC#4, P126, V7-LSB |
| CD102 | ICAM-2, Ly60 |
| CD103 | HML-1, alpha6, integrin alphaE |
| CD104 | beta4 integrin, TSP1180, ITGB4, TSP-180 |
| CD105 | Endoglin, HHT1, ORW, SH-2 |
| CD106 | VCAM-1 |
| CD107a | LAMP-1 |
| CD107b | LAMP-2 |
| CD108 | SEMA7A, JMH blood group antigen |
| CD109 | 8A3, E123 7D1, 150kD TGF-b-1-binding protein, |
| | Platelet-specific Gov antigen |
| CD110 | MPL, TPO-R |
| CD111 | PRR1, Nectin-1 |
| CD112 | PRR2, Nectin-2 |
| CD113 | PVRL3, Nectin3 |
| CD114 | G-CSFR |
| CD115 | M-CSFR, c-fms |
| CD116 | GM-CSFRalpha |
| CD117 | c-kit, SCFR |
| CD118 | LIFR, gp190 |
| CD119 | IFNgammaR |
| CD120a | TNFR-I |
| CD120b | TNFR-II |
| CD121a | IL-1R type I |
| CD121b | IL-1R, type II |
| CD122 | IL-2Rbeta |
| CD123 | IL-3R |
| CD124 | IL-4R |
| CD125 | IL-5R |
| CD126 | IL-6R |
| CD127 | IL-7R |
| CD130 | gp130, IL6ST, IL6-b or CD130 |
| CD131 | CSF2RB, IL3RB, IL5RB, CDw131 |
| CD132 | Common gamma chain, IL-2Rg |
| CD133 | AC133, prominin-like 1 |
| CD134 | OX-40 |
| CD135 | Flt3/Flk2 |
| CD136 | MSP-R, RON |
| CD137 | 4-1BB |
| CD138 | Syndecan-1 |
| CD139 | |
| CD140a | PDGFRalpha |
| CD140b | PDGFRbeta |
| CD141 | Thrombomodulin |
| CD142 | Tissue Factor |
| CD143 | ACE |
| CD144 | VE-Cadherin, Cadherin-5 |
| CD145 | |
| CD146 | MUC18, S-endo |
| CD147 | Neurothelin, basoglin |
| CD148 | HPTP-eta |
| CD150 | SLAM |
| CD151 | PETA-3 |
| CD152 | CTLA-4 |
| CD153 | CD30L |
| CD154 | CD40L, gp39, TRAP |
| CD155 | PVR |
| CD156a | ADAM8 |
| CD156b | TACE/ADAM 17 |
| CD156c | ADAM10 |
| CD157 | BST-1 |
| CD158a | p58.1 |
| CD158b | p58.2 |
| CD159a | NKG2A |
| CD159c | NKG2C |
| CD160 | BY55 |
| CD161 | NKR-P1A |
| CD162 | PSGL-1 |
| CD162R | PEN-5 |
| CD163 | 130kD |
| CD164 | MGC-24 |
| CD165 | AD2, gp37 |
| CD166 | ALCAM |
| CD167a | DDR1 |
| CD168 | RHAMM |
| CD169 | sialoadhesin, Siglec-1 |
| CD170 | Siglec-5, CD33-1ike2 |
| CD171 | L1CAM, HSAS, HSAS1, MASA, MIC5, |
| | N-CAML1, S10, SPG1, NILE |
| CD172a | SIRPgamma |
| CD172b | SIRPbeta, SIRB1 |
| CD172g | SIRPgamma, SIRPB2 |
| CD173 | Blood group H type 2 |
| CD174 | Lewis Y, FUT3, Les, FT3B |
| CD175 | Tn |
| CD175s | Sialyl-Tn |
| CD176 | Thomson Friedrenreich Ag |
| CD177 | NB1 |
| CD178 | FasL, CD95L |
| CD179a | V pre B |
| CD179b | Lambda 5 |
| CD180 | RP-105 |
| CD181 | CXCR1, IL-8RA |
| CD182 | CXCR2, IL-8RB |
| CD183 | CXCR3 |
| CD184 | CXCR4, fusin |
| CD185 | CXCR5, BLR1 |
| CD186 | CXCR6, BONZO |
| CD191 | CCR1, MIP-1alphaR, RANTES-R |
| CD192 | CCR2, MCP-1-R |
| CD193 | CCR3, CKR3 |
| CD195 | CCR5 |
| CD196 | CCR6, LARC receptor, DRY6 |
| CD197 | CCR7 |
| CD198 | CCR8, GPRCY6, TER1 |
| CD199 | CCR9, GPR-9-6 |
| CD200 | OX-2 |
| CD201 | EPC-R |
| CD202b | Tie2, Tek |
| CD203c | NPP3/PDNP3, ENpp1, PD-1b |
| CD204 | Macrophage scavenger-R |
| CD205 | DEC-205 |
| CD206 | macrophage mannose-R |
| CD207 | Langerin, C-type Lectin domain family 4 |
| | member K (CLEC4K) |
| CD208 | DC-LAMP |
| CD209 | DC-SIGN |
| CD210 | IL-10-R |
| CD212 | IL-12-R beta1 |
| CD213a1 | IL-13-R alpha1 |
| CD213a2 | IL-13-R a1pha2 |
| CD217 | IL-17-R |
| CD218a | IL-18Ralpha, IL-1Rrp |
| CD218b | IL-18Rbeta, IL18RAP |
| CD220 | Insulin-R |
| CD221 | IGF-1 R |
| CD222 | IGF-II R, mannose-6 phosphate-R |
| CD223 | LAG-3 |
| CD224 | GGT |
| CD225 | Leu-13 |
| CD226 | DNAM-1, PTA-1, TLiSA1 |
| CD227 | MUC1, EMA |
| CD228 | Melanotransferrin |
| CD229 | Ly-9 |
| CD230 | Prion protein |
| CD231 | TALLA-1, A15 |
| CD232 | VESP-R |
| CD233 | Band 3, SLC4A1 |
| CD234 | Duffy, DARC |
| CD235a | Glycophorin A |
| CD235ab | Glycophorin A/B |
| CD235b | Glycophorin B |
| CD236 | Glycophorin C/D |
| CD236R | Glycophorin C |
| CD238 | Kell blood group glycoprotein (Kel), Kell blood |
| | group antigen, Endothelin-3-converting |
| | enzyme (ECE3) |
| CD239 | Basal cell adhesion molecule (BCAM, B-CAM), |
| | Lutheran blood group glycoprotein, |
| | Lutheran blood group antigen (Lu) |
| CD240CE | Rh30CE |
| CD240D | Rh30D |
| CD241 | RhAg, Rh50 |
| CD242 | ICAM-4 |
| CD243 | MDR-1, p170, P-gp |
| CD244 | 2B4, NAIL, NKR2B4, Nmrk, SLAMF4 |
| CD245 | p220/240 |
| CD246 | ALK, Ki-1 |
| CD247 | CD3-z, CD3H, CD3Q, CD3Z, T3Z, TCRZ |
| CD248 | TEM1, Endosialin |
| CD249 | Aminopeptidase A |
| CD252 | TNFSF4, GP34, OX4OL, TXGP1, CD134L, |
| | OX-40L, OX40L |
| CD253 | TRAIL, Apo-2L, TL2, TNFSF10 |
| CD254 | TRANCE, RANKL, OPGL |
| CD256 | APRIL, TALL-2 |
| CD257 | BLyS, BAFF, TALL-1 |
| CD258 | LIGHT, HVEM-L |
| CD261 | TRAIL-R1, DR4 |
| CD262 | TRAIL-R2, DR5 |
| CD263 | TRAIL-R3, DcR1, LIT |
| CD265 | RANK, TRANCE-R, ODFR |
| CD266 | TWEAK-R, FGF-inducible 14 |
| CD267 | TACI, TNFR SF13B |
| CD268 | BAFFR, TR13C |
| CD269 | BCMA, TNFRSF13B |
| CD271 | NGFR, p75 (NTR) |
| CD272 | BTLA |
| CD273 | B7DC, PD-L2, PDCD1L2 |
| CD274 | B7-H1, PD-L1 |
| CD275 | B7-H2, ICOSL, B7-RP1, GL50 |
| CD276 | B7-H3 |
| CD277 | BT3.1, butyrophilin SF3 A1, BTF5 |
| CD278 | ICOS, AILIM |
| CD279 | PD1, SLEB2 |
| CD280 | ENDO180, UPARAP |
| CD281 | TLR1 |
| CD282 | TLR2 |
| CD283 | TLR3 |
| CD284 | TLR4 |
| CD289 | TLR9 |
| CD292 | BMPR1A, ALK3 |
| CD293 | BMPR1B, ALK6 |
| CD294 | CRTH2. GPR44 |
| CD295 | LeptinR, LEPR |
| CD296 | ART1, RT6, ART2 |
| CD297 | ART4, dombrock blood group |
| CD298 | Na+/K+ −ATPase beta3 subunit |
| CD299 | DC-SIGN-related, LSIGN, DC-SIGN2 |
| CD300a | CMRF35H, IRC1, IRp60 |
| CD300c | CMRF35A, LIR |
| CD300e | CMRF35L |
| CD301 | MGL, HML |
| CD302 | DCL1, BIMLEC |
| CD303 | BDCA2, HECL |
| CD304 | BDCA4, neuropilin 1 |
| CD305 | LAIR1 |
| CD306 | LAIR2 |
| CD307a | FCRL1, IRTA5 |
| CD307b | FCRL2, IRTA4 |
| CD307c | FCRL3, IRTA3 |
| CD307d | FCRL4, IRTA1 |
| CD307e | FCRL5, IRTA2 |
| CD309 | VEGFR2, KDR |
| CD312 | EMR2 |
| CD314 | NKG2D, KLR |
| CD315 | CD9P1, SMAP6, FPRP, PTGFRN |
| CD316 | EWI2, PGRL, CD81P3, KASP |
| CD317 | BST2, HM1.24 |
| CD318 | CDCP1, SIMA135 |
| CD319 | CRACC, SLAMF7 |
| CD320 | 8D6A, 8D6 |
| CD321 | JAM1, F11 receptor |
| CD322 | JAM2, VE-JAM |
| CD324 | E-Cadherin, Uvomorulin |
| CD325 | N-Cadherin, NCAD |
| CD326 | Ep-CAM, Ly74 |
| CD327 | SIGLEC6 |
| CD328 | SIGLEC7, AIRM-1 |
| CD329 | SIGLEC9 |
| CD331 | FGFR1, Fms-like tyrosine kinase-2, KAL2, |
| | N-SAM |
| CD332 | FGFR2, BEK, KGFR |
| CD333 | FGFR3, ACH, CEK2 |
| CD334 | FGFR4, JTK2, TKF |
| CD335 | NKp46, Ly-94 homolog |
| CD336 | NKp44, Ly-95 homolog |
| CD337 | NKp30, Ly117 |
| CD338 | ABCG2, BCRP, Bcrpl, MXR |
| CD339 | Jagged-1, JAG1, JAGL1, hJ1 |
| CD340 | ERB-B2, Neu, Her-2 |
| CD344 | FZD4, Frizzled homolog 4 |
| CD349 | FZD9, Frizzled homolog 9 |
| CD350 | FZD10, Frizzled homolog 10 |
| CD351 | FCAMR, Fc receptor, IgA, IgM, high affinity |
| CD352 | SLAMF6, Ly108, NTB-A |
| CD353 | SLAMF8, BLAME |
| CD354 | TREM1 |
| CD355 | CRTAM, Cytotoxic and regulatory T-cell |
| | molecule |
| CD357 | TNFRSF18, Tumor necrosis factor receptor |
| | superfamily, member 18, GITR |
| CD358 | TNFRSF21, Tumor necrosis factor receptor |
| | superfamily, member 21, DR6 |
| CD359 | PI16 |
| CD360 | IL21R |
| CD361 | EVI2B (ectoptic viral integration site 2B) |
| CD362 | Syndecan-2 |
| CD363 | S1PR1, Sphingosine-1-phosphate receptor 1, |
| | EDG-1 |

In some embodiments, the biomarker the biomarker can include or exclude, for example: CD1, CD2, CD3, CD4, CD5, CD6, CD7, CD8, CD9, CD10, CD11a, CD11b, CD11c, CD13, CD14, CD15, CD16, CD19, CD20, CD21, CD22, CD23, CD25, CD30, CD33, CD34, CD38, CD41, C43, CD45, CD56, CD57, CD58, CD61, CD64, C71, CD79a, CD99, CD103, CD117, CD123, CD138, CD138, CD163, CD235a, HLA-DR, Kappa, Lambda, Pax-5, BCL-2, Ki-67, ZAP-70, MPO, TdT, FMC-7, Pro2PSA, ROMA (HE4+CA-125), OVA1 (multiple proteins), HE4, Fibrin/fibrinogen degradation product (DR-70), AFP-L3%, Circulating Tumor Cells (EpCAM, CD45, cytokeratins 8, 18+, 19+), HER2, NEU, Prostate stem cell antigen (PSCA), epithelial-specific antigen (ESA), epithelial cell adhesion molecule (EpCAM), α2β1, VEGFR-1, VEGFR-2, CD133, AC133 antigen, p63 protein, c-Kit, CA19-9, Estrogen receptor (ER), Progesterone receptor (PR), Pro2PSA, HER-2/neu, CA-125, CA15-3, CA27.29, Free PSA, Thyroglobulin, Nuclear Mitotic Apparatus protein (NuMA, NMP22), Alpha-fetoprotein (AFP)b, ROMA (HE4+CA-125), OVAL HE4, DR-70, p63 protein, c-Kit, CA19-9, Total PSA, alpha-Methylacyl-CoA Racemase/AMACR, CA125/MUC16, ER alpha/NR3A1, ER beta/NR3A2, Thymidine Kinase 1, AG-2, BRCA1, BRCA2, CA15-3/MUC-1, Caveolin-1, CD117/c-kit, CEACAM-5/CD66e, Cytokeratin 14, EGF R/ErbB1, HIN-1/SCGB3A1, Ki-67/MKI67, MKP-3, Nestin, NGF R/TNFRSF16, NM23-H1, PARP, PP4, Serpin E1/PAI-1, 14-3-3 beta, 14-3-3 sigma, 14-3-3 zeta, 15-PGDH/HPGD, 5T4, A33, ABCBS, ABCB6, ABCG2, ACE/CD143, ACLP, ACP6, Afadin/AF-6, Afamin, AG-2, AG-3, Akt, Aldo-keto Reductase 1C3/AKR1C3, alpha 1B-Glycoprotein, alpha 1-Microglobulin, AlphaB Crystallin/CRYAB, alpha-Fetoprotein/AFP, alpha-Methylacyl-CoA Racemase/AMACR, AMFR/gp78, Annexin A3, Annexin A8/ANXA8, APC, Apolipoprotein A-I/ApoA1, Apolipoprotein A-II/ApoA2, Apolipoprotein E/ApoE, APRIL/TNFSF13, ASCL1/Mash1, ATBF1/ZFHX3, Attractin, Aurora A, BAP1, Bcl-2, Bcl-6, beta 2-Microglobulin, beta-1,3-Glucuronyltransferase 1/B3GAT1, beta-Catenin, beta-III Tubulin, Bikunin, BMI-1, B-Raf, BRCA1, BRCA2, Brk, C4.4A/LYPD3, CA15-3/MUC-1, c-Ab1, Cadherin-13, Caldesmon/CALD1, Calponin 1, Calretinin, Carbonic Anhydrase IX/CA9, Catalase, Cathepsin D, Caveolin-1, Caveolin-2, CBFB, CCR7, CCR9, CEACAM-19, CEACAM-20, CEACAM-4, CHD1L, Chitinase 3-like 1, Cholecystokinin-B R/CCKBR, Chorionic Gonadotropin alpha Chain (alpha HCG), Chorionic Gonadotropin alpha/beta (HCG), CKAP4/p63, Claudin-18, Clusterin, c-Maf, c-Myc, Coactosin-like Protein 1/CotL1, COMMD1, Cornulin, Cortactin, COX-2, CRISP-3, CTCF, CTL1/SLC44A1, CXCL17/VCC-1, CXCL8/IL-8, CXCL9/MIG, CXCR4, Cyclin A1, Cyclin A2, Cyclin D2, Cyclin D3, CYLD, Cyr61/CCN1, Cytokeratin 14, Cytokeratin 18, Cytokeratin 19, DAB2, DCBLD2/ESDN, DC-LAMP, Dkk-1, DLL3, DMBT1, DNMT1, DPPA2, DPPA4, E6, E-Cadherin, ECM-1, EGF, EGF R/ErbB1, ELF3, ELTD1, EMMPRIN/CD147, EMP2, Endoglin/CD105, Endosialin/CD248, Enolase 2/Neuron-specific Enolase, EpCAM/TROP1, Eps15, ER alpha/NR3A1, ER beta/NR3A2, ErbB3/Her3, ErbB4/Her4, ERCC1, ERK1, ERK5/BMK1, Ets-1, Exostosin 1, EZH2, Ezrin, FABP5/E-FABP, Fascin, FATP3, FCRLA, Fetuin A/AHSG, FGF acidic, FGF basic, FGF R3, FGF R4, Fibrinogen, Fibroblast Activation Protein alpha/FAP, Follistatin-like 1/FSTL1, FOLR1, FOLR2, FOLR3, FOLR4, FosB/GOS3, FoxMl, FoxO3, FRAT2, FXYD5/Dysadherin, GABA-A R alpha 1, GADD153, GADD45 alpha, Galectin-3, Galectin-3BP/MAC-2BP, gamma-Glutamylcyclotransferase/CRF21, Gasl, Gastrin-releasing Peptide R/GRPR, Gastrokine 1, Gelsolin/GSN, GFAP, GLI-2, Glutathione Peroxidase 3/GPX3, Glypican 3, Golgi Glycoprotein 1/GLG1, gp96/HSP90B1, GPR10, GPR110, GPR18, GPR31, GPR87, GPRC5A, GPRC6A, GRP78/HSPA5, HE4/WFDC2, Heparanase/HPSE, Hepsin, Her2, HGF R/c-MET, HIF-2 alpha/EPAS1, HIN-1/SCGB3A1, HLA-DR, HOXB13, HOXB7, HSP70/HSPA1A, HSP90, Hyaluronidase 1/HYAL1, ID1, IgE, IGFBP-2, IGFBP-3, IGFBP-4, IGFBP-6, IGF-I, IGF-I R, IGF-II, IGFL-3, IGFLR1, IL-1 beta/IL-1F2, IL-17E/IL-25, IL-2, IL-6, IMP Dehydrogenase 1/IMPDH1, Importin alpha 2/KPNA2, ING1, Integrin beta 1/CD29, Integrin beta 3/CD61, IQGAP1, Isocitrate Dehydrogenase 1/IDH1, ITIH4, ITM2C, Jagged 1, JNK, JunB, JunD, Kallikrein 2, Kallikrein 6/Neurosin, KCC2/SLC12A5, Ki-67/MKI67, KiSS1R/GPR54, KLF10, KLF17, L1CAM, Lactate Dehydrogenase A/LDHA, Lamin B1, LEF1, Leptin/OB, LIN-28A, LIN-28B, Lipocalin-2/NGAL, LKB1/STK11, LPAR3/LPA3/EDG-7, LRMP, LRP-1B, LRRC3B, LRRC4, LRRN1/NLRR-1, LRRN3/NLRR-3, Ly6K, LYPD1, LYPD8, MAP2, Matriptase/ST14, MCAM/CD146, M-CSF, MDM2/HDM2, Melan-A/MART-1, Melanocortin-1 R/MC1R, Melanotransferrin/CD228, Melatonin, Mer, Mesothelin, Metadherin, Metastin/KiSS1, Methionine Aminopeptidase, Methionine Aminopeptidase 2/METAP2, MFAP3L, MGMT, MIA, MIF, MINA, Mind Bomb 2/MIB2, Mindin, MITF, MKK4, MKP-1, MKP-3, MMP-1, MMP-10, MMP-13, MMP-2, MMP-3, MMP-8, MMP-9, MRP1, MRP4/ABCC4, MS4A12, MSH2, MSP R/Ron, MSX2, MUC-4, Musashi-1, NAC1, Napsin A, NCAM-1/CD56, NCOA3, NDRG1, NEK2, NELL1, NELL2, Nesfatin-1/Nucleobindin-2, Nestin, NFkB2, NF-L, NG2/MCSP, NGF R/TNFRSF16, Nicotinamide N-Methyltransferase/NNMT, NKX2.2, NKX3.1, NM23-H1, NM23-H2, Notch-3, NPDC-1, NTS1/NTSR1, NTS2/NTSR2, OGR1, Olig2, Osteopontin/OPN, Ovastacin, OXGR1/GPR80/P2Y15, p130Cas, p15INK4b/CDKN2B, p16INK4a/CDKN2A, p18INK4c/CDKN2C, p21/CIP1/CDKN1A, p27/Kip1, P2X5/P2RX5, p53, PARP, PAUF/ZG16B, PBEF/Visfatin, PDCD4, PDCD5, PDGF R alpha, PDGF R beta, PDZD2, PEA-15, Pepsinogen A5/PGA5, Peptidase Inhibitor 16/PI16, Peroxiredoxin 2, PGCP, PI 3-Kinase p85 alpha, PIWIL2, PKM2, PLK1, PLRP1, PP4, P-Rex1, PRMT1, Profilin 1, Progesterone R B/NR3C3, Progesterone R/NR3C3, Progranulin/PGRN, Prolactin, Prostaglandin E Synthase 2/PTGES2, PSAP, PSCA, PSMA/FOLH1/NAALADase 1, PSMA1, PSMA2, PSMB7, PSP94/MSMB, PTEN, PTEN, PTH1R/PTHR1, PTK7/CCK4, PTP beta/zeta/PTPRZ, Rab25, RARRES1, RARRES3, Ras, Reg4, Ret, RNF2, RNF43, S100A1, S100A10, S100A16, S100A2, S100A4, S100A6, S100A7, S100A9, S100B, S100P, SART1, SCUBE3, Secretin R, Serpin A9/Centerin, Serpin E1/PAI-1, Serum Amyloid A1, Serum Amyloid A4, SEZ6L, SEZ6L2/BSRP-A, Skp2, SLC16A3, SLC45A3/Prostein, SLC5A5, SLC5A8/SMCT1, SLC7A7, Smad4, SMAGP, SOCS-1, SOCS-2, SOCS-6, SOD2/Mn-SOD, Soggy-1/DkkL1, SOX11, SOX17, SOX2, SPARC, SPARC-like 1/SPARCL1, SPINK1, Src, STEAP1, STEAP2, STEAP3/TSAP6, STRO-1, STYK1, Survivin, Synaptotagmin-1, Syndecan-1/CD138, Syntaxin 4, Synuclein-gamma, Tankyrase 1, Tau, TCF-3/E2A, TCL1A, TCL1B, TEM7/PLXDC1, TEM8/ANTXR1, Tenascin C, TFF1, TGF-beta 1, TGF-beta 1, 2, 3, TGF-beta 1/1.2, TGF-beta 2/1.2, TGF-beta RI/ALK-5, THRSP, Thymidine Kinase 1, Thymosin beta 10, Thymosin beta 4, Thyroglobulin, TIMP Assay Kits, TIMP-1, TIMP-2, TIMP-3, TIMP-4, TLE1, TLE2, TLR2, TM4SF1/L6, TMEFF2/Tomoregulin-2, TMEM219, TMEM87A, TNF-alpha, TOP2A, TopBP1, t-Plasminogen Activator/tPA, TRA-1-60(R), TRA-1-85/CD147, TRAF-4, Transgelin/TAGLN, Trypsin 2/PRSS2, Tryptase alpha/TPS1, TSPAN1, UBE2S, uPAR, u-Plasminogen Activator/Urokinase, Urotensin-II R, VAP-1/AOC3, VCAM-1/CD106, VEGF, VEGF R1/Flt-1, VEGF R2/KDR/Flk-1, VEGF/P1GF Heterodimer, VSIG1, VSIG3, YAP1, ZAG, ZAP70, ZMIZ1/Zimp10, and Carcino-embryonic antigen. In some embodiments, the biomarker can include or exclude, for example, the proteins listed in the Cancer Atlas (http://www.proteinatlas.org/search/cancer).

In some embodiments, the biomarker is selected from markers expressed by kidney cells, infectious or parasitic agents, solid tumor cells, circulating tumor cells, or any other cell useful for diagnosis or prognosis. In some embodiments, the biomarker is selected from markers expressed on the surface or within kidney cells, infectious agents (e.g., bacteria or virus), solid tumor cells, or circulating tumor cells. In some embodiments, the biomarkers expressed on the surface or within kidney cells can include or exclude, for example: KIM-1, Albumin, beta-2 microglobulin, Cystatin C, Clusterin, Apolipoprotein A-I/ApoA1, CXCL8/IL-8, ERCC1, Ki-67/MKI67, MMP-9, or Trefoil factor-3.

In some embodiments, the biomarker is one or a plurality of markers for a particular type of cancer. In some embodiments, the biomarker for breast cancer can include or exclude her2-neu, ER, PR, Ki-67, and p53. In some embodiments, the biomarker for lung cancer can include or exclude TTF-1, Napsin A, CK 5/6, p40/63, and Synaptophosmin. In some embodiments, the biomarker for prostrate cancer can include or exclude AMACR, PSA, CEA, and p63. In some embodiments, the biomarker for colorectal cancer can include or exclude MLH1, MSH2, PMS2, MSH6, c-Kit, p16, and BRAF V600E. In some embodiments, the biomarker for tumor infiltrating lymphocytes can include or exclude CD4, CD8, CD14, CD20, CD45RO, FoxP3, PD-L, and PD-L1. In some embodiments, the biomarker for cancers of the urinary system (bladder, kidney, urethra) can include or exclude CK7, p63, CK20, p53, Ki-67, PSA, Vimentin, and PAX8.

In some embodiments, the biomarker is cell-specific. The cells can be in a healthy state (normal) or diseased state (abnormal). Monocytes and macrophages can exhibit a biomarker that includes or excludes the CD14 and CD16 biomarkers. Lymphocyte B cells can exhibit a biomarker that includes or excludes the CD20 biomarker. Lymphocyte NK cells can exhibit a biomarker that includes or excludes the CD56 biomarker. Lymphocytes T cells can exhibit a biomarker that includes or excludes the CD3 biomarker. T Reg cells can exhibit a biomarker that includes or excludes the CD4, CD25, and FoxP3 biomarkers. Cytotoxic T cells can exhibit a biomarker that includes or excludes the CD8 biomarker. Helper T cells can exhibit a biomarker that includes or excludes the CD4 biomarker. Naïve T cells can exhibit a biomarker that includes or excludes the CD45RA biomarker. Memory T cells can exhibit a biomarker that includes or excludes the CD45RO biomarker. Tth cells can exhibit a biomarker that includes or excludes the CXR5 biomarker. Th17 cells can exhibit a biomarker that includes or excludes the CCR6 biomarker. Th2 cells can exhibit a biomarker that includes or excludes the CCR4 biomarker. Th1 cells can exhibit a biomarker that includes or excludes the CXCR3 biomarker. Tumor cells can exhibit a biomarker that includes or excludes the PanCK biomarker.

The term “biomarker-binding moiety” as used herein is a moiety that can specifically bind to a biomarker. In some embodiments, the biomarker-binding moiety can include or exclude, for example, an antibody or antibody fragment, nanobody, receptor fragment, DNA aptamer, DNA/RNA oligonucleotide, RNA aptamer, PNA aptamer, peptide aptamer, LNA aptamer, carbohydrate, or a lectin.

The term “antibody” as used herein is a protein that can specifically bind to an antigen. In some embodiments, an antibody can include or exclude, for example, any recombinant or naturally occurring immunoglobulin molecule such as a member of the IgG class e.g. IgG1 and also any antigen binding immunoglobulin fragment, such as Fv, Fab and F(ab′)2 fragments, antibody fragment, ScFv (single-chain variable fragment, a fusion protein of the variable regions of the heavy (V_{H}) and light chains (V_{L}) of immunoglobulins, connected with a short linker peptide of ten to about 25 amino acids), or single-domain antibody (nanobody), and any derivatives thereof. In embodiments where the biomarker binding moiety comprises an antibody, the antibody can be a monoclonal or polyclonal antibody.

The term “antibody fragments” as used herein, refers to a portion of an intact antibody, wherein the portion retains at least one, and as many as most or all, of the functions normally associated with that portion when present in an intact antibody. In some embodiments, an antibody fragment comprises an antigen binding site of the intact antibody and thus retains the ability to bind antigen. In some embodiments, an antibody fragment, for example one that comprises the Fc region, retains at least one of the biological functions normally associated with the Fc region when present in an intact antibody, such as FcRn binding, antibody half-life modulation, ADCC function and complement binding. In some embodiments, an antibody fragment is a monovalent antibody that has an in vivo half-life substantially similar to an intact antibody. For example, such an antibody fragment may comprise an antigen binding arm linked to an Fc sequence capable of conferring in vivo stability to the fragment.

“Polyclonal Antibodies” or “PAbs,” are heterogeneous populations of antibody molecules derived from the sera of animals immunized with an antigen, or an antigenic functional derivative thereof. For the production of polyclonal antibodies, host animals such as rabbits, mice and goats, may be immunized by injection with an antigen or antigen-conjugate, optionally supplemented with adjuvants. Polyclonal antibodies may be unpurified, purified or partially purified from other species in an antiserum. The techniques for the preparation and purification of polyclonal antibodies are described in various general and more specific references, including but not limited to Kabat & Mayer, Experimental Immunochemistry, 2d ed., (Thomas, Springfield, Ill. (1961)); Harlow & Lane, Antibodies: A Laboratory Manual (Cold Spring Harbor Laboratory Press, Cold Spring Harbor, N.Y. (1988)); and Weir, Handbook of Experimental Immunology, 5th ed. (Blackwell Science, Cambridge, Mass. (1996)).

“Monoclonal antibodies,” or “MAbs,” are homogeneous populations of antibodies to a particular antigen and may be obtained by any technique that provides for the production of antibody molecules, such as by continuous culture of cell lines. These techniques include, but are not limited to the hybridoma technique of Köhler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad. Sci. USA, 80:2026-30 (1983)), and the EBV-hybridoma technique (Cole, et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the MAb of this invention may be cultivated in vitro or in vivo. Production of high titers of MAbs in vivo makes this a presently preferred method of production.

Techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody can be a molecule in which different portions are derived from different animal species, such as those having a variable region derived from a murine MAb and a human immunoglobulin constant region.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce gene-single chain antibodies suitable for use in the present invention. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)2 fragments that can be produced by pepsin digestion of the antibody molecule and the Fab fragments that can be generated by reducing the disulfide bridges of the F(ab′)₂ fragments. Alternatively, Fab expression libraries may be constructed (Huse, et al., Science, 246:1275-81 (1989)) to allow rapid and easy identification of monoclonal Fab fragments with the desired specificity.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

The term “humanized” forms of non-human (e.g., murine) antibodies are chimeric antibodies that comprise minimal sequence derived from non-human immunoglobulin. In some embodiments, a humanized antibody is a human immunoglobulin (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, the biomarker binding moiety may bind to a peptide, protein, protein fragment, glycosylation moiety or pattern, or a carbohydrate. The biomarker binding moiety can be selected from a biomarker binding moiety, e.g., an antibody or fragment thereof or other biomarker binding moiety that binds to any of the biomarkers in Table 1. In some embodiments, the biomarker binding moiety can be selected from a biomarker binding moiety that binds to, for example: CD1, CD2, CD3, CD4, CD5, CD6, CD7, CD8, CD9, CD10, CD11a, CD11b, CD11c, CD13, CD14, CD15, CD16, CD19, CD20, CD21, CD22, CD23, CD25, CD30, CD33, CD34, CD38, CD41, C43, CD45, CD56, CD57, CD58, CD61, CD64, C71, CD79a, CD99, CD103, CD117, CD123, CD138, CD138, CD163, CD235a, HLA-DR, Kappa, Lambda, Pax-5, BCL-2, Ki-67, ZAP-70, MPO, TdT, FMC-7, Pro2PSA, ROMA (HE4+CA-125), OVA1 (multiple proteins), HE4, Fibrin/fibrinogen degradation product (DR-70), AFP-L3%, Circulating Tumor Cells (EpCAM, CD45, cytokeratins 8, 18+, 19+), HER2, NEU, Prostate stem cell antigen (PSCA), epithelial-specific antigen (ESA), epithelial cell adhesion molecule (EpCAM), α2β1, VEGFR-1, VEGFR-2, CD133, AC133 antigen, p63 protein, c-Kit, CA19-9, Estrogen receptor (ER), Progesterone receptor (PR), HER-2/neu, CA-125, CA15-3, CA27.29, Free PSA, Thyroglobulin, Nuclear Mitotic Apparatus protein (NuMA, NMP22), Alpha-fetoprotein (AFP)b, Total PSA, and Carcino-embryonic antigen, or any of the biomarkers described herein. In some embodiments, when the biomarker-binding moiety is anti-CD45, the biomarker signature obtained is the white blood cell count.

### Optical Contrast Agents

In some embodiments, the contacting the cells with an optical contrast agent can comprise adding a dye or colorant to the cells. In some embodiments, the optical contrast agent can be a leuco dye, cell stain, or any dye useful for imaging for morphological analysis including, for example, any dye useful for histological, cytological, cytopathological, or histopathological imaging. In some embodiments, the optical contrast agent provides visual classification and identification of cells by differentially staining cells. The histopathological imaging can be imaging method used in a treatment or diagnostic clinic. The leuco dye can be red leuco dye, methylene blue, crystal violet, phenolphthalein, thymolphthalein, or methylene green.

In some embodiments, the optical contrast agent can be a cell stain selected from: Giemsa stain, Wright stain, Wright-Giemsa stain, May-Grünwald stain, Mallory trichrome, Periodic acid-Schiff reaction stain, Weigert's elastic stain, Heidenhain's AZAN trichrome stain, Orcein stain, Masson's trichrome, Alcian blue stain, May-Grünwald-Giemsa, van Gieson stain, Hansel stain, Reticulin Stain, Gram stain, Bielschowsky stain, Ferritin stain, Fontana-Masson stain, Hales colloidal iron stain, Pentachrome stain, Azan stain, Luxol fast blue stain, Golgi's method (reduced silver), reduced gold, Chrome alum/haemotoxylin stain, Isamin blue stain, Argentaffin stains, Warthin-Starry silver stain, Nissl stain, Sudan Black and osmium stain, osmium tetroxide stain, hematoxylin stain, Uranyl acetate stain, lead citrate stain, Carmine stain, safranin stain, and Ziehl-Neelsen stain.

In some embodiments, the optical contrast agent can be a dye or colorant selected from: eosin Y, eosin B, azure B, pyronin G, malachite green, toluidine blue, copper phthalocyanin, alcian blue, auramine-rhodamine, acid fuschin, aniline blue, orange G, acid fuschin, neutral red, Sudan Black B, acridine orange, Oil Red O, Congo Red, Fast green FCF, Perls Prussian blue reaction, nuclear fast red, alkaline erythrocin B, and naphthalene black.

In some embodiments, the cells labelled with the optical contrast agent can be selectively decolored. The selective decloration can comprise removal of the stain, conversion of the stain or dye to a colorless form, or degradation of the dye. The stain can be removed by washing. The washing can be done in the presence of a different pH than during contacting with the stain such that the overall charge of the stained proteins changes thereby affecting removal of the stain. The washing can be done with a solvent system which solubilizes the stain at the altered pH. In some embodiments, optical contrast agent can be a leuco dye. In some embodiments, the leuco dye can be converted to a colorless form by the addition of one or more electrons to the dye. Electrons can be added to the dye via a reduction method. The reduction method can be effected by an electrochemical reduction, photoreduction, or reaction with a reducing agent. The reducing agent can by sodium cyanoborohydride sodium borohydride, NADH (formed in situ or separately added), ascorbic acid (and salts thereof, for example sodium ascorbate, potassium ascorbate, ammonium ascorbate, etc.) or dithiothreitol (DTT). In some embodiments, the leuco dye can be converted to a colored form by the removal of one or more electrons from the dye. One or more electrons can be removed from the dye by an oxidation method. The oxidation method can be effected by an electrochemical oxidation, photooxidation, or reaction with an oxidation agent. In some embodiments, the oxidation agent can be NAD+, NADP+, pyruvate, acetaldehyde, cystine, alpha-ketoglurate, ibquinone, 2 cytochrome c, 2 cytochrome c, 2 cytochrome a3, or oxygen.

When the sample is from tissue, the optical contrast agent can be a H&E (hematoxylin and eosin) stain. In one aspect, the optical contrast agent may be suitable for supravital staining. In one aspect, the optical contrast agent may be suitable for vital staining.

Cell Morphology from Imaging the Optical Contrast Agent

Histopathological analysis often involves imaging of a sample contacted with an optical contrast agent. In some aspects, cellular morphological features can be identified by the visual characteristics of a cell, including or excluding, for example, the size, shape, or the presence and/or absence of colored internal bodies. In some embodiments, the imaging of the morphological features of the contacted cells can comprise measuring an optical property of the optical contrast agent. The optical property of the optical contrast agent can include or exclude, for example, absorbance, scattering, fluorescence, photoluminesence, Raman emission, and photoluminescent lifetime. In a preferred embodiment, the optical property detected by the optical contrast agent is the absorbance of light. The wavelength of the absorbed light can be from the ultraviolet range to the infrared range. Preferably, the wavelength of absorbed light is in the visible range (300-800 nm). The optical property of the optical contrast agent can be measured under a microscope with either a light field illumination or dark field illumination.

In some embodiments, the morphological features identified from the cell can comprise the cell surface shape, the cell nucleus shape, the chromatin shape, the nucleolar shape, the number of nucleolus, the grade of the cancer (closeness to a normal cell), the arrangement of the cells, or combinations of the foregoing. In some embodiments, the morphological features identified from the cell of a subject can be compared against a previously obtained cell of a subject so as to determine if the cells exhibit dysplasia over time.

Morphologically, a cancerous cell is characterized by a large nucleus, having an irregular size and shape, prominent nucleoli, and scarce and intensely colored or pale cytoplasm. Changes in cell nucleus over time can be imaged of the cells surface, volume, nucleus/cytoplasm ratio, shape, density, structure and homogeneity. Other morphological features of a cell that can be imaged are characteristics are related to nucleus segmentation, invaginations, changes in chromatin, such as heterochromatin reduction, increase of interchromatin and perichromatin granules, increase of nuclear membrane pores, and the formation of inclusions, etc. The nucleolus of a cancer cell can be characterized by hypertrophy, macro- and microsegregation, its movement towards the membrane, numerical increase and formation of intranuclear canalicular systems between the nuclear membrane and the nucleolus. In some embodiments, malignant cancer cells can exhibit mitoses. In a malignant cancer cell, the number of mitoses can increase, with an atypical mitosis forming with defects in the mitotic spindle appear, which results in triple or quadruple asters (cellular structures shaped like a star, comprised of microtubules, formed around each centrosome during mitosis) and dissymmetrical structures and atypical forms of chromosomes. In some embodiments, a cancerous cell may exhibit nuclear changes that can explain the presence of different cell clones and genetic anomalies associated with these changes.

The cytoplasm of a cancerous cell can also change, with the appearance of new structures appear or disappearance of normal structures. In some embodiments, the new structures in cancer cells can be cytoplasmic inclusions. Cytoplasmic inclusions can include Auer rods, clumps of stainable cellular granular material that form elongated needles seen in the cytoplasm of leukemic blasts (partially differentiated cells). In some forms of neoplasms, apoptosis occurs, with the presence of apoptotic bodies in the cytoplasm.

Malignant cancer cells have a small cytoplasmic amount, frequently with vacuoles.

In cancerous cells, the granular endoplasmic reticulum may exhibit a simplified structure appearance. The ER may be amorphous, with granular or filamentous material accumulating in the cisternae. In some embodiments, fragmentation and degranulation can be observed, with the interruption of connections between the granular endoplasmic reticulum and mitochondria. A decrease of the granular endoplasmic reticulum from tumor cells can occur with an increase of free ribosomes and polysomes.

In cancerous cells, the Golgi apparatus can be poorly developed, which indicates a lack of tumor cell differentiation. Cancerous cells that have completely lost differentiation may exhibit a Golgi apparatus.

Cancer cell mitochondria can decrease in volume with tumor development. Mitochondria can show a high variability of shape and volume, with very large mitochondria observed. Cancerous cell mitochondrial crystals can be different from those of a normal cell, with inclusions and pyknotic images present in the matrix.

A cancer cell may exhibit secondary lysosomes, myelinic structures and lipofuscin granules.

A cancer cell membrane can exhibit an increase or diminution in the number of surface receptors, changing cell sensitivity to the regulating mechanisms of the host; structural changes of proteins or surface receptors that no longer react with the corresponding ligand; and the presence of new surface molecules, characteristic of the embryonic tissue, which are hidden at the surface of adult cells. Abnormal surface molecules are able to act as antigens and are recognized by the mechanisms of humoral and cellular defense. Tumor cells can be covered with immune complexes, which allows the complement to destroy the cells covered by antibodies and allows phagocytes to attack the opsonized cells. In some embodiments, the immune complexes can comprise a biomarker.

In some embodiments, the distribution of receptors on the cell surface in malignant cells is altered, which modifies the cell agglutination behavior.

In some embodiments, the method for detecting the biomarker-morphological profile of a cell can further comprise: (h) diagnosing the subject's condition based on the biomarker-morphological profile of each cell. In some aspects the subject's condition may include presence of a hematological cancer, non-malignant hematological disorder, solid tumor, kidney disease, bladder disease, liver disease, or infectious disease. The hematological cancer can be leukemia, lymphoma, or multiple myeloma. The non-malignant hematological disorder can be anemia or sickle cell disease. The solid tumor can be breast cancer, lung cancer, prostate cancer, bone cancer, colorectal cancer, or bladder cancer. When the solid tumor is breast cancer, the biomarkers can be, for example, Her2 or Neu. In some embodiments, the kidney disease can be acute kidney injury, chronic kidney disease, lupus nephritis, kidney rejection, or preeclampsia. In some embodiments, the infectious disease can be HIV, hepatitis, sexually transmitted diseases, or sepsis. In some embodiments, the hematological cancer can further comprise circulating cancer cells.

In some embodiments, when the subject's condition is a cancer, the subject's condition can be further identified by the lineage of the malignancy. For example, the lineage of the malignancy can be negative, Myeloid line, Lymphoid T cell line, or Lymphoid B cell line.

Contacting the Cells with Functionalized Nanoparticles

The term “contacting” as used herein, refers generally to providing access of one component, reagent, analyte or sample to another. For example, contacting can involve mixing a solution comprising a functionalized nanoparticle with a sample comprising a cell. The solution comprising one component, reagent, analyte or sample may also comprise another component or reagent, such as dimethyl sulfoxide (DMSO) or a detergent, which facilitates mixing, interaction, uptake, or other physical or chemical phenomenon advantageous to the contact between components, reagents, analytes and/or samples, in some embodiments of the invention, contacting involves adding a solution comprising a functionalized nanoparticle to a sample comprising a cell utilizing a delivery apparatus, such as a pipette-based device or syringe-based device.

The cells can be reacted with functionalized nanoparticles so as to create a functionalized nanoparticle-cell complex. In some embodiments, the cells can be contacted with one or a plurality of functionalized nanoparticle species by subjecting the cells and functionalized nanoparticles to an external force to increase the local concentration of the functionalized nanoparticles and cells. The external force can be a gravitational, electric, or magnetic force. The gravitational force can be generated by centrifugation. The centrifugation can be pulsed. The pulse duration can be 10 seconds, 20 seconds, 30 seconds, 40 seconds, 50 seconds, 1 minute, or 2 minutes. The pulse duration can be from 1 minute to 2 minutes, 2 minutes to 3 minutes, 3 minutes to 4 minutes, 4 minutes to 5 minutes, or any time period inbetween the aforementioned times. The magnetic force can be effected by paramagnetic nanoparticles, wherein the core of the nanoparticle comprises a paramagnetic region and the shell of the nanoparticle can include or exclude Ag, Au, Pt, Pd, Rh, Ro, Al, Cu, Ru, Cr, Cd, Zn, Si, Se or mixtures or alloys thereof. The paramagnetic region can be comprised of magnetic iron oxide (Fe₂O₃). In some embodiments, charged species can be added to the solution comprising cells before contacting the cells with the functionalized nanoparticles so as to prevent agglomeration or enhance functionalized nanoparticle penetration into the cell. In some embodiments, the charged species can be charged polymers, which can be added to the cells after first providing a sample comprising cells from a subject.

When functionalized nanoparticles are subject to a high gravitational force, they can irreversibly agglomerate. In some embodiments, charge-neutral organics can be added to the cells during the contacting with the functionalized nanoparticles to prevent/reduce/inhibit functionalized nanoparticle agglomeration. The charge-neutral organics can be a solvent with a high dielectric constant or a charge-neutral surfactant. In some embodiments, the charge-neutral surfactants can include or exclude, for example, Tween, Brij, Span, IGEPAL, MERPOL, Triton or Pluronic surfactant. In some aspects, the Tween surfactants can include or exclude, for example, Tween20, Tween40, Tween60, or Tween85. In some aspects, the Pluronic surfactants can include or exclude, for example, Pluronic 408, Pluronic P-123, Pluronic F-68, Pluronic F-127, Pluronic L31, Pluronic L35, Pluronic F-108, Pluronic, L-61, Pluronic L-81, Pluronic L-64, Pluronic L-121, Pluronic 10R5, Pluronic 17R4, Pluronic 31R1, or Pluronic 188. In some aspects, the Brij surfactants can include or exclude, for example, Brij 52, Brij 58, Brij C10, Brij L4, Brij O10, Brij S10, Brij S20, or Brij S100. In some aspects, the IDEPAL surfactants can include or exclude, for example, IGEPAL CA-520, IGEPAL CA-720, IGEPAL CO-520, IGEPAL CO-630, IGEPAL CO-720, IGEPAL CO-890, or IGEPAL DM-970. In some aspects, the Span surfactant can be Span40. In some aspects, the MERPOL surfactant can include or exclude, for example, MERPOL DA, MERPOL HCS, MERPOL OJ, MERPOL SE, MERPOL SE, or MERPOL A. In some aspects, the Triton surfactant can include or exclude, for example, Triton X-100, Triton X-114, or Triton X-405. In some aspects, the surfactant can be sorbitan monooleate or sorbitan monopalmitate. In some aspects, the solvent with a high dielectric constant can include or exclude, for example, DMSO (dimethylsulfoxide), DMF (N,N-dimethylformamide), THF (tetrahydrofuran), ethanol, isopropanol, or any n-alcohol wherein n is from 3 to 8.

### Resonant Light Scattering Detection of Functionalized Nanoparticles

The term “detecting” as used herein refers to any method of verifying the presence of a given nanoparticle or particle. The techniques used to accomplish this may include, but are not limited to resonance light scattering or plasmon resonance.

Resonance light scattering is a physical phenomenon where a particle with a diameter less than the wavelength of incident light exhibits a surface plasmon wave around the particle and said wave becomes coherent to the circumference of the particle. Particle electrons can resonate in phase with the incident light forming an electromagnetic dipole that emits energy as scattered light. The wavelength of the reflected (scattered) light is a function of the composition, shape, and particle size. In some embodiments, the composition of the particle can be a noble metal, such as gold or silver. In some embodiments, the size of the particle is below the wavelength of white light (below 300 nm).

The scattered light from a particle exhibiting a resonance light scattering effect can be used as the signal for ultrasensitive analyte detection. (Yguerabide, J., et al., Analytical Biochemistry, 262; 137-156 (1998)). The advantages of using particles with a wavelength less than the wavelength of light is that (a) the particles can be detected at concentrations at low concentrations in suspension by eye and a simple illuminator, such as with dark field illumination, (b) the particles as a light source do not photobleach, (c) the color of scattered light can be changed by changing particle size or composition for multicolor multiplexing, and (d) the particles can be conjugated with biomarker-binding moieties for specific analyte detection.

In some aspects, the resonant light scattering from each observed complexed nanoparticle can be detected using evanescent or non-evanescent light. In some aspects, the non-evanescent light can be transmitted light. The resonant light scattering of the complexed nanoparticle can be detected when imaging under a dark field illumination. In some aspects, an illuminated slide holder can replace the darkfield condenser in the microscope. The illuminated slide holder can use total internal reflection (TIRF) to illuminate the slide holder. TIRF illumination can eliminate or reduce the scatter from other light scattering elements on the substrate surface. TIRF illumination will not interact with such surface debris as transilluminated darkfield illumination would.

The TIRF illuminated slide holder can be analyzed by TIRF microscopy. The inventors have recognized that utilizing TIRF microscopy in the present disclosure reduces background fluorescence from outside the focal plane and can noticeably improve the signal-to-noise ratio, and thus the spatial resolution of the resonance light scattered from the nanoparticles. TIRF microscopy utilizes an induced evanescent wave in a limited substrate region immediately adjacent to the interface between two media having different refractive indices. In some embodiments, the utilized TIRF interface can be the contact area between the substrate and a glass coverslip or tissue culture container. The illuminated slide holder can comprise optical fibers to deliver light to the edge to the slide. The illuminated slide holder can be the Darklite Vertical Illuminator (Micro Video Instruments, Inc, Avon, Mass.).

In some embodiments, the dark field illuminator can provide effective low-angle lighting to targeted regions of the substrate. The dark field illuminator can be comprised of LEDs (light emitting diodes). The LEDs can be positioned so as to provide low-angle illumination to provide a high contrast image. In some embodiments, the dark field illuminator can be the DF-50, DF-150, DF-200 illuminators from Microscan Systems, Inc. (Renton, Wash.).

In some embodiments, the dark field microscopy system can comprise a system with high NA (numerical aperature) condensors, with non-evanescent illumination, vibration reduction, and stray light reduction to improve dark-field performance. In some embodiments, the inverted darkfield contrast system described in U.S. Pat. No. 6,704,140, herein incorporated by reference in its entirety, can be used.

In some embodiments, the illuminated slide holder can be illuminated by transmitted light. In some embodiments, the illuminated slide holder can be illuminated by epi-illumination. In some embodiements, the source of the epi-illumination can be from a laser.

In some embodiments, the interrogation time can be adjusted to detect all of the nanoparticles while minimizing the saturation of any particular nanoparticle species. Some nanoparticles may exhibit a bloom effect when the interrogation time is too long. In some embodiments, detection of the resonant light scattering from some of the observed complexed nanoparticles can be completed in under, for example, 1 second, 500 milliseconds, 200 milliseconds, 100 milliseconds, 50 milliseconds, 25 milliseconds, 10 milliseconds, 5 milliseconds, 2 milliseconds, 1 millisecond, or 0.2 milliseconds. In some embodiments, detection of the resonant light scattering from some of the observed complexed nanoparticles can be completed in under, for example, from 2 seconds to 1 second, 1 second to 500 milliseconds, 500 milliseconds to 200 milliseconds, 200 milliseconds to 100 milliseconds, 100 milliseconds to 50 milliseconds, 50 milliseconds to 25 milliseconds, 25 milliseconds to 10 milliseconds, 10 milliseconds to 5 milliseconds, 5 milliseconds to 2 milliseconds, 2 milliseconds to 1 millisecond, 1 millisecond to 0.2 milliseconds, or any time between any of the foregoing time periods. In some embodiments, the dark field illumination can comprise LEDs with different wavelengths. The different wavelengths can be applied in parallel or in series. When the different wavelengths are applied in series, the interrogation time can be varied for each different LED wavelength. In some embodiments, detection of the resonant light scattering from some of the observed complexed nanoparticles can be completed in under, for example, 1 second, 500 milliseconds, 200 milliseconds, 100 milliseconds, 50 milliseconds, 25 milliseconds, 10 milliseconds, 5 milliseconds, 2 milliseconds, 1 millisecond, or 0.2 milliseconds when one LED wavelength is applied, then completed in under, for example, 1 second, 500 milliseconds, 200 milliseconds, 100 milliseconds, 50 milliseconds, 25 milliseconds, 10 milliseconds, 5 milliseconds, 2 milliseconds, 1 millisecond, or 0.2 milliseconds, when a different LED wavelength is applied. In some embodiments, a software control system can adjust the detection time, compare the detections of two or more interrogations, normalize the relative intensities for two or more different nanoparticles when interrogated two or more times, and/or normalize for binding kinetics and/or strengths of functionalized nanoparticals to their targets.

In some embodiments, the illumination can comprise one or a plurality of signal exposures. There can be, for example, 1, 2, 3, 4, or 5 signal exposures. Each signal exposure can be for a different time. A software control system can adjust the detection time, compare the detections of two or more interrogations, and/or normalize the relative intensities for two or more different nanoparticles when interrogated two or more times.

In some embodiments, the one or a plurality of functionalized nanoparticle species can comprise nanoparticles from 5 to 200 nm in diameter. In some embodiments, the nanoparticles can include or exclude sizes of 5, 7, 10, 12, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 nanometers (nm) in diameter. In some embodiments, the nanoparticles can include or exclude sizes from 4 to 6 nm, 6 to 8 nm, 9 to 11 nm, 11 to 13 nm, 14 to 16 nm, 19 to 21 nm, 24 to 26 nm, 29 to 31 nm, 34 to 36 nm, 39 to 41 nm, 44 to 46 nm, 49 to 51 nm, 54 to 56 nm, 59 to 61 nm, 64 to 66 nm, 69 to 71 nm, 74 to 76 nm, 79 to 81 nm, 84 to 86 nm, 89 to 91 nm, 94 to 96 nm, 99 to 101 nm, 104 to 106 nm, 109 to 111 nm, 119 to 121 nm, 124 to 126 nm, 129 to 131 nm, 134 to 136 nm, 139 to 141 nm, 144 to 146 nm, 149 to 151 nm, 154 to 156 nm, 159 to 161 nm, 164 to 166 nm, 169 to 171 nm, 174 to 176 nm, 179 to 181 nm, 184 to 186 nm, 189 to 191 nm, 194 to 196 nm, 199 to 201 nm, or in between any of the foregoing sizes. In some embodiments, the size distribution of the nanoparticles can be less than 25% coefficient of variation (CV), less than 20% CV, 15% CV, less than 10% CV, less than 5% CV, or less than 4%, 3%, 2% or less than 1% CV, or any range between any two of the recited percentages. In some embodiments, the diameter can be measured at the maximum difference between the sides of the particle or the minimum distance between the sides of the particle, when viewed from a side profile. In some embodiments, the nanoparticles can be made from any metal or metal composition as described herein.

In some embodiments, each nanoparticle preparation has a narrow size distribution. By narrow size distribution is meant that an individual nanoparticle preparation has a scattering spectrum whose full-width half maximum ranges from 5 to 150 nm. (See Chen et. al, Journal of Biomedical Optics 10(2), 024005 (March/April 2005)). In some embodiments, an individual nanoparticle preparation has a scattering spectrum whose full-width half maximum ranges from 5 to 50 nm. In some embodiments, a spectrum of light scattering is collected for each pixel in the imaged field. Next, the spatial distribution of each molecular target is represented by the spatial distribution of nanoparticles, which in turn is reported by the presence and/or absence of its resonant light scattering peak at each pixel. The size distribution can be combined with the compositional variation of each nanoparticle preparation to achieve greater multiplexing capacity.

In some embodiments, the nanoparticles can be comprised of a noble metal. The nanoparticles can be comprised metals that can include or exclude Ag, Au, Pt, Pd, Rh, Ro, Al, Cu, Ru, Cr, Cd, Zn, Si, Se or mixtures or alloys thereof. The alloy can be an alloy of gold (Au) and silver (Au). In some embodiments, the alloy can be of Copper (Cu) and Gold (Au) to modulate the intensity of the reflected light (see Su, Y. et al., Nanoscale Research Letters, 8:408, 2013). In some embodiments, the composition of the alloy can be adjusted to affect the intensity of the reflected light. In some embodiments, the alloy composition can be adjusted to modulate the wavelength of the reflected light. The nanoparticles can comprise mixtures of the listed metals in discrete shells or layers. For example, a nanoparticle may be comprised of an Au core and a Si or SiO₂ (silica) shell. In some embodiments, the core can be Fe₂O₃. In some embodiments, the nanoparticles are spherical, tubular, cylindrical, pyramidal, cubic, egg-shaped, t-bone-shaped, urchin- or rose-like (with spiky uneven surfaces) or hollow shaped. In some embodiments, the nanoparticles may have a round, oval, triangular, square, egg-shaped, or a t-bone-shaped cross-section.

In some embodiments the nanoparticles can comprise a chemical group to which other functional groups can be added, such as streptavidin, biotin, amino-functionalized dextran, a biomarker binding moiety or an oligonucleotide or other component of a releasable or displaceable nanoparticle system. The chemical group can be, for example, lipoic acid, reduced forms of lipoic acid, an amine, carboxylic acid, an alkyne, an azide, or —NHS.

In some embodiments, the nanoparticles can be the size of any nanoparticles described herein.

In some embodiments, the nanoparticles can include or exclude Pt 30, 50, or 70 nm (Nanocomposix#'s PTCN30-25M, PTCN50-25M, PTCN70-25M); Ag 5, 10, 20, 30, 40, 50, 60, 70, 75, 80, 100, or 200 nm (Nanocomposix # AGCN5-25M, AGCN10-25M, AGCN20-25M, AGCN30-25M, AGCN40-25M, AGCNO5-25M, AGCN60-25M, AGCN70-25M, AGCN75-25M, AGCN80-25M, AGCN100-25M, or AGCN200-25M); Au 5, 7, 10, 12, 15, 20, 30, 40, 50, 60, 70, 80, or 100 nm (Nanocomposix # AUCN5-25M, AUCN7-25M, AUCN10-25M, AUCN12-25M, AUCN15-25M, AUCN20-25M, AUC30-25M, AUCN40-25M, AUCN50-25M, AUCN60-25M, AUCN70-25M, AUCN80-25M, or AUCN100-25M); Ag 75 nm or 100 nm nanocubes (Nanocomposix # SCPH75-1M, SCPH100-1M); 550 nm resonant Ag nanoplates (Nanocomposix # SPPN550-25M); 650 nm resonant Ag nanoplates (Nanocomposix # SPPN650-25M); 750 nm resonant Ag nanoplates (Nanocomposix # SPPN750-25M); 850 nm resonant Ag nanoplates (Nanocomposix # SPPN850-25M); 950 nm resonant Ag nanoplates (Nanocomposix # SPPN950-25M); 1050 nm resonant Ag nanoplates (Nanocomposix # SPPN1050-25M); 1150 nm resonant Ag nanoplates (Nanocomposix custom order); 660 nm resonant Au nanoshells (Nanocomposix # GSPN660-25M), 800 nm resonant Au nanoshells (Nanocomposix # GSPN800-25M), 980 nm resonant Au nanoshells (Nanocomposix # GSPN980-25M); 660 nm resonant Au nanorods (Nanocomposix # GRCN660-10M), 800 nm resonant Au nanorods (Nanocomposix # GRCN800-10M), 980 nm resonant Au nanorods (Nanocomposix # GRCN980-10M); 30 nm Au50Ag50 alloy (50/50) (Cytodiagnostics # GSA-30-20), 30 nm Au80Ag20 alloy (80/20) (Cytodiagnostics # GSB-30-20), 30 nm Au20Ag80 (20/80) (Cytodiagnostics # GSC-30-20); gold nanorods from Cytodiagnostics (25 nm diam, 650 nm max abs.—GRC3K-25-650-25); functionalized and non-functionalized Nanourchins (Cytodiagnotics)—50 nm (GU-50-20), 60 nm (GU-60-20), 70 nm (GU-70-20), 80 nm (GU-80-20), 90 nm (GU-90-20), and 100 nm (GU-100-20).

In some embodiments, the nanoparticles can exhibit a peak resonance wavelength of the nanoparticle plasmon resonance from 240 to 1150 nm. In some embodiments, the nanoparticles can exhibit a peak resonance wavelength of the nanoparticle plasmon resonance from 400 to 900 nm.

In some embodiments, the plurality of functionalized nanoparticle species can be from 2 to 347 different species of functionalized nanoparticle species. In some embodiments, the up to 347 functionalized nanoparticle species, may comprise up to 50 different types of nanoparticles, and each plurality of functionalized nanoparticle species may comprise 50 functionalized nanoparticle species. In some embodiments, the plurality of functionalized nanoparticle species can be from 2 to 50 different species of functionalized nanoparticle species. In some embodiments, the plurality of functionalized nanoparticle species can be from 2 to 10 different species of functionalized nanoparticle species. In some embodiments, the plurality of functionalized nanoparticle species can be from 2 to 5 different species of functionalized nanoparticle species.

In some embodiments, each species of functionalized nanoparticle species can be functionalized with a different species of biomarker-binding moiety. In some embodiments, each species of functionalized nanoparticle species is functionalized with a different biomarker binding moiety.

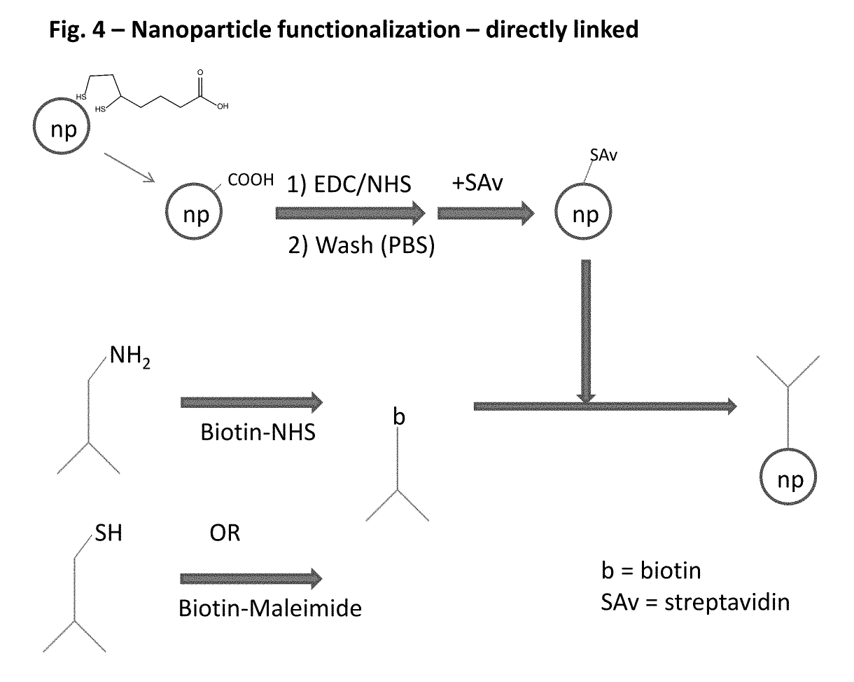

In some embodiments, the nanoparticles can be functionalized by using streptavidin-biotin binding. FIGS. 4, 5, and 6 depict some of the embodiments by which the nanoparticles can be functinonalized. In some embodiments, nanoparticles coated with a carboxylic acid functional group can be activated with EDC/NHS (1-Ethyl-3-(3-dime thylaminopropyl)carbodiimide/N-hydroxy-succinimide), followed by a wash to yield an EDC-functionalized nanoparticle, via the process depicted in FIG. 4. Other amide coupling agents can be used instead of EDC, for example, DCC (dicyclohexylcarbodiimide), EDAC.HCl, (N-(3-Dimethylaminopropyl)-N′-ethylcarbodiimide.HCl), HOBt (1-Hydroxybenzotriazole), HOOBt (HODhbt) (Hydroxy-3,4-dihydro-4-oxo-1,2,3-benzotriazine), HOAt (1-Hydroxy-7-aza-1H-benzotriazole), DMAP (4-(N,N-Dimethylamino)pyridine), BOP (Benzotriazol-1-yloxy-tris(dimethylamino)-phosphonium hexafluorophosphate), PyBOP (Benzotriazol-1-yloxy-tripyrrolidinophosphonium hexafluorophosphate), PyOxim (Ethyl cyano(hydroxyimino)acetato-O2)-tri-(1-pyrrolidinyl)-phosphonium hexafluorophosphate), PyBrOP (7-Aza-benzotriazol-1-yloxy-tripyrrolidinophosphonium hexafluorophosphate), DEPBT (3-(Diethoxy-phosphoryloxy)-1,2,3-benzo[d]triazin-4(3H)-one), TBTU/HBTU (2-(1H-Benzotriazol-1-yl)-N,N,N′,N′-tetramethylaminium tetrafl uoroborate/hexafluorophosphate), HCTU ((2-(6-Chloro-1H-benzotriazol-1-yl)-N,N,N′,N′-tetramethylaminium hexafluorophosphate), HDMC (N-[(5-Chloro-1H-benzotriazol-1-yl)-dimethylamino-morpholino]-uronium hexafluorophosphate N-oxide), HATU (2-(7-Aza-1H-benzotriazol-1-yl)-N,N,N′,N′-tetramethylaminium hexafluorophosphate), COMU (1-[1-(Cyano-2-ethoxy-2-oxoethylideneaminooxy)-dime thylamino-morpholino]-uronium hexafluorophosphate), TOTT ((2-(1-Oxy-pyridin-2-yl)-1,1,3,3-tetramethylisothiouronium tetrafluoroborate), TFFH (Tetramethylfluoroformamidinium hexa-fluorophosphate), EEDQ (N-Ethoxycarbonyl-2-ethoxy-1,2-dihydroquinoline), T3P (2-Propanephosphonic acid anhydride), DMTMM (4-(4,6-Dimethoxy-1,3,5-triazin-2-yl)-4-methylmorpholinium salt), or CDI (1,1′-Carbonyldiimidazole). In some alternative embodiments, the coupling can be performed in the presence of a base. The base can be organic or inorganic. The inorganic bases can include or exclude, for example, carbonate buffer, or phosphate buffer. The organic bases can be triethylamine, Diisopropylethylamine (DIPEA), or N-methylmorpholine (NMM).

The wash can be a pH mild wash so as to not hydrolyze the NHS moieties. The mild pH wash can be with PBS buffer (phosphate buffered saline, pH around 7.4). Next, streptavidin can be reacted with the EDC-functionalized nanoparticle to yield a streptavidin-functionalized nanoparticle. Other avidin-like molecules can be used in place of streptavidin, for example: avidin, neutravidin, superavidin, and streptavidin with one, two or three biotins already bound. In some embodiments, the biomarker binding moiety can be functionalized with a biotin. In some embodiments, the biomarker binding moiety is an antibody. The antibody can be reacted with a cross-linker, such as Sulfo-SMCC (Pierce) followed by a thiol-conjugated biotin to yield a biotinylated antibody. In an alternative embodiment, the antibody can be reacted with a NHS-conjugated biotin, where the NHS-conjugated biotin can react with any free amine on the antibody (prefeable, free amines from lysine residues) to yield a biotinylated antibody. In an alternative embodiment, the antibody can be reacted with DTT (dithioerithritol) to break the di-thiol cysteine bond to yield a free sulfuryl hydryl group. The sulfuryl hydryl group can be reacted with a maleimide-conjugated biotin to yield a biotinylated antibody. In some embodiments, the nanoparticle can be purchased with a functional group selected from: carboxylic acid, NHS, streptavidin, amine, alkyne, or aldehyde. The biotinylated antibody can be reacted to the streptavidin-functionalized nanoparticle to create the directly-linked antibody-functionalized nanoparticle.

The terms “polynucleotide” and “nucleic acid (molecule)” are used interchangeably to refer to polymeric forms of nucleotides of any length. The polynucleotides may comprise deoxyribonucleotides, ribonucleotides and/or their analogs. Nucleotides may have any three-dimensional structure, and may perform any function, known or unknown. The term “polynucleotide” includes single-stranded, double-stranded and triple helical molecules. “Oligonucleotide” refers generally to polynucleotides of between 5 and about 100 nucleotides of single- or double-stranded nucleic acid, typically DNA. Oligonucleotides are also known as oligomers or oligos and may be isolated from genes, or synthesized (e.g., chemically or enzymatically) by methods known in the art. A “primer” refers to an oligonucleotide, usually single-stranded, that provides a 3′-hydroxyl end for the initiation of enzyme-mediated nucleic acid synthesis. The following are non-limiting embodiments of polynucleotides: a gene, a gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine.

Sugar modifications (e.g., 2′-o-methyl, 2-fluor and the like) and phosphate backbone modifications (e.g., morpholino, PNA′, thioates, dithioates, methyl phosphonates, and the like) can be incorporated singly, or in combination, into the nucleic acid molecules of the present invention. In some embodiments, for example, a nucleic acid of the invention may comprises a modified sugar and a modified phosphate backbone. In another embodiment, a nucleic acid of the invention may comprise modifications to sugar, base and phosphate backbone.

The nucleotide sequence of the nucleic acids of the present invention is of less importance than the functional roles they are required to perform. Accordingly, the sequence of the nucleic acids, as well as the length of the nucleic acid component of the binding pair, may vary considerably, provided the nucleic acid component of the binding pair can still perform the functional roles they are required to perform. Importantly, the sequence and length of the nucleic acids of the binding pair are not limited to those exact sequences and lengths of the exemplary binding pairs disclosed herein. The nucleic acids of the binding pair thus can be of different lengths and or sequence. An important function of the nucleic acid component of the binding pairs of the present invention is to provide a linker between the biomarker-binding moiety and the functionalized nanoparticle by the ability to hybridize with a complementary strand of the nucleic acid to form a nucleic acid duplex.

The stability of a nucleic acid duplex is dependent in part on the length of the region of complementarity between the nucleic acid strands in the duplex. A longer complementarity region or overlap between nucleic acids increases the stability of the duplex that is formed. Conversely, a shorter overlap leads to a less stable duplex. The stability of a duplex can be measured as a function of the melt temperature, Tm, where a highly stable duplex results in a high Tm and a less stable duplex results in a lower Tm. Nucleic acids of the present invention are designed to have defined stability that can be manipulated by altering length, temperature, backbone composition, base pair selection, base pair structure, sugar structure, solvent and other conditions. Factors that influence the stability of the hybrid include, but are not limited to, the concentration of the nucleic acid-labeled binding pairs, salt concentration, temperature, organic solvents such as ethanol, DMSO, tetramethylammonium ions (TMA⁺), base pair mismatches and the like.

In some embodiments, the backbone composition of an oligonucleotide can be varied to produce an oligonucleotide with a selected relative duplex strength. An oligonucleotide backbone resulting in a more stable duplex can be selected from: peptide-nucleic acids (PNA), locked nucleic acids (LNA), or normal deoxyribonucleic acid (DNA). An oligonucleotide backbone resulting in a less stable duplex can be selected from: unlocked nucleic acids, methyl phosphonate, or thiophosphonates. PNAs have a peptide-backbone rather than a ribose-phosphate backbone of normal DNA. The PNA backbone is composed of repeating N-(2-aminoethyl)-glycine units linked by peptide bonds. The purine and pyrimidine bases are linked to the PNA backbone by a methylene bridge (—CH2-) and a carbonyl group (—(C═O)—), The PNA backbone thus lacks charged phosphate groups. PNAs are not easily recognized by either native nucleases or proteases, imbuing them resistance to enzymatic degradation and pH stability. The LNA backbone comprises a ribose moiety which is modified with an extra bridge connecting the 2′ oxygen and 4′ carbon locking the ribose in the 3′-endo (North) conformation. The locked ribose conformation enhances base stacking and backbone pre-organization, significantly increases duplex stability of LNA/DNA duplexes. Methyl phosphonate backbones replace the charged anionic phosphate with a neutral methyl phosphonate ester. The resulting decrease in backbone charge results in a less stable duplex relative to a normal DNA backbone, yet also confers resistance to nuclease activity. Thiophosphonate backbones comprise a non-bridging oxygen on the phosphate backbone to form a phosphorothioate (PS) linkage. Thiophosphonate backbones exhibit nuclease resistance and a less stable duplex relative to a normal DNA backbone.

In some embodiments, the duplex stability can be adjusted by incorporating one or a plurality of non-natural base pairs. In some embodiments, the non-natural base can be iso-G or iso-C, as described in Richert, C., et J. Am. Chem. Soc. 118, 4518-4531 (1996), herein incorporated by reference. In some embodiments, the non-natural base can be diflurotoluene, as described in Schweitzer, B. A., et al., J. Am. Chem. Soc. 117, 1863-1872 (1995), herein incorporated by reference. In some embodiments, the non-natural base can be MMO2 or SICS, as described in Leconte, A. M. et al. J. Am. Chem. Soc. 130, 2336-2343 (2008), herein incorporated by reference. In some embodiments, the non-natural base can be Ds or Dioll-Px, as described in Yamashige, R. et al. Nucl. Acids Res. 40, 2793-2806 (2012), herein incorporated by reference. In some embodiments, the non-natural base can be P or Z, as described in Yang; Z., et al., J. Am. Chem. Soc. 133, 15105-15112(2011), herein incorporated by reference. In some embodiments, the non-natural base can be NaM or 5SICS, as described in Malyshev, D. A. et al. Proc. Natl Acad. Sci. USA 109, 12005-12010 (2012), herein incorporated by reference.

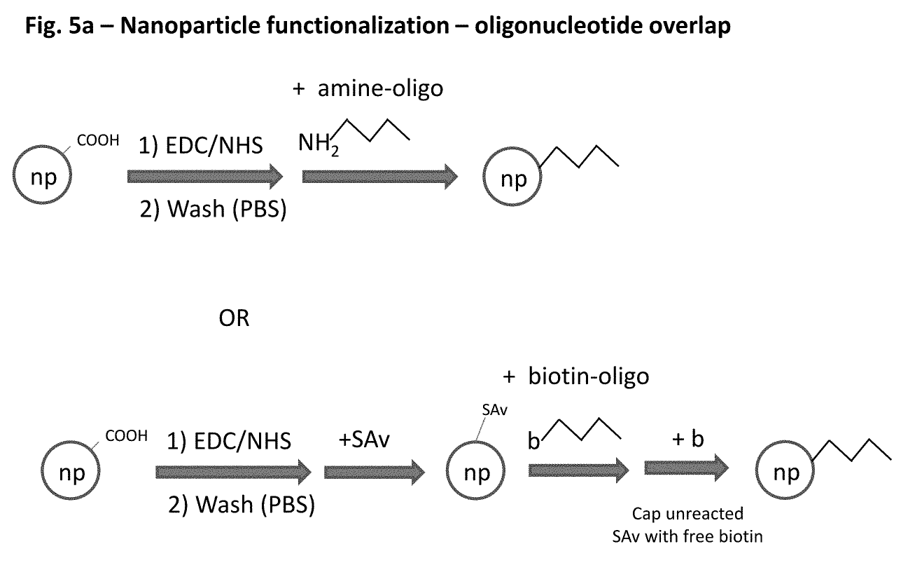

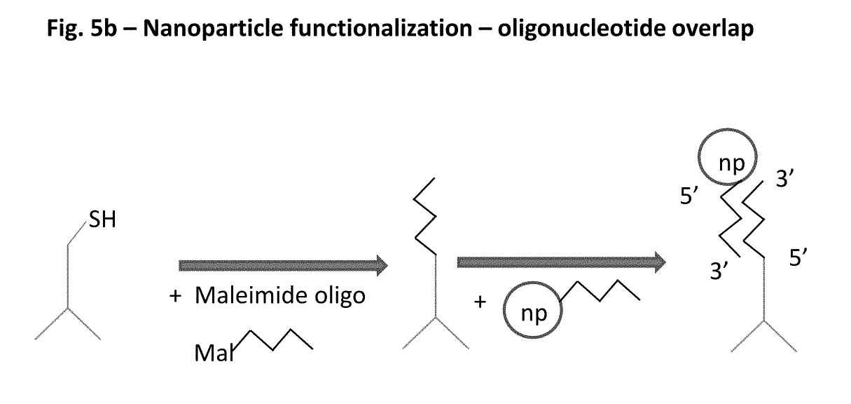

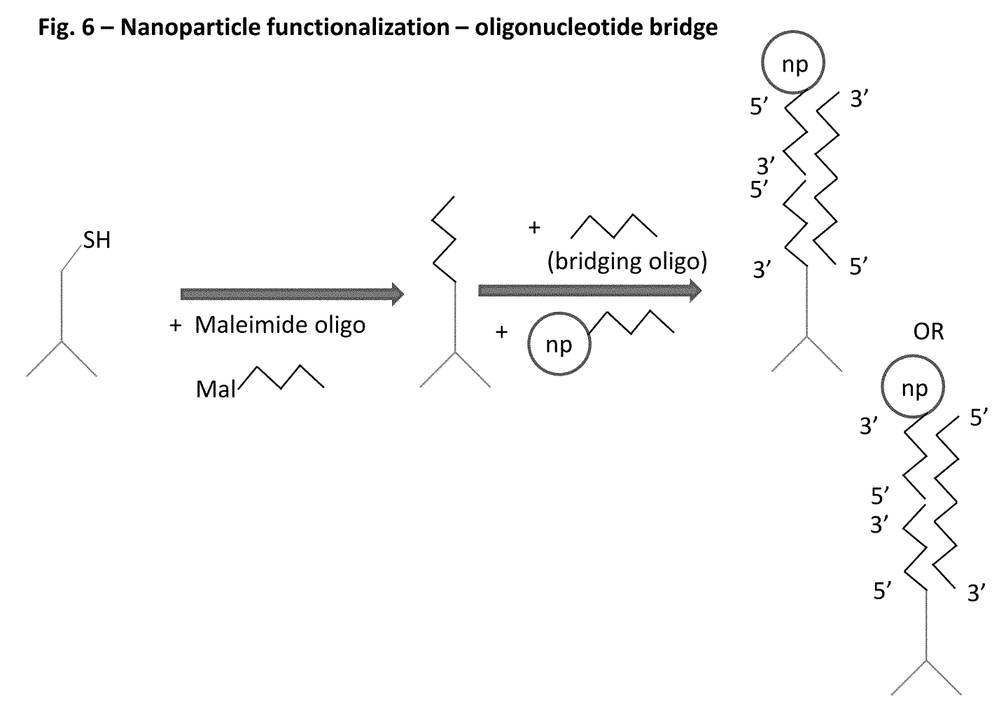

In some embodiments, the nanoparticle can be functionalized by functionalized with a first oligonucleotide, by the process depicted in FIG. 4. The carboxylic acid-functionalized nanoparticle can be reacted with EDC/NHS followed by a mild pH wash. Then, the EDC-functionalized nanoparticle can be reacted to an amino-functionalized oligonucleotide to yield an oligonucleotide-functionalized nanoparticle, as shown in FIG. 5. In some embodiments, unreacted EDC groups can be reacted to prevent cross-talk with other functionalized nanoparticle species when the species are mixed by reacting the unreacted EDC with a small molecule amine. The small molecule amine can be ethanolamine. In another embodiment, the EDC-functionalized nanoparticle can be reacted to streptavidin to yield a streptavidin-functionalized nanoparticle. The streptavidin-functionalized nanoparticle can be reacted with a biotin-modified oligonucleotide to yield an oligonucleotide-functionalized nanoparticle. Unreacted streptavidin can be blocked by adding free biotin before mixing one species of oligonucleotide-functionalized nanoparticles with other species of oligonucleotide-functionalized nanoparticles. In some embodiments, the biomarker binding moiety can be functionalized with a second oligonucleotide, as shown in FIG. 5. In some embodiments, the biomarker binding moiety is an antibody. The antibody can be reacted with DTT (dithioerythritol) to yield free sulfurylhydryl groups. The sulfuryl hydryl groups can be reacted with a maleimide-functionalized second oligonucleotide. In some embodiments, the second oligonucleotide conjugated to the functionalized biomarker binding moiety (e.g., antibody) can comprise a portion complementary to a portion of the first oligonucleotide and hybridization of the first oligonucleotide to the second oligonucleotide forms a linker comprising a double-stranded nucleic acid. In some embodiments, the first and second oligonucleotides can both comprise a portion complementary to a portion of a third oligonucleotide which can act as a bridging oligonucleotide, as shown in FIG. 6. Modified oligonucleotides discussed herein can be used with the modifier at the 3′ or 5′ terminus. When the first oligonucleotide is conjugated to the nanoparticle at the 5′ terminus, the corresponding terminus of the second oligonucleotide is selected such that the two oligonucleotides are complementary in the proper orientation if directly hybridized or indirectly hybridized by a bridging oligonucletide.

In some embodiments, when the nanoparticle comprises a silica (SiO₂) shell, the nanoparticle can be functionalized with a functionalized silane. The silane can be dissolved in an organic solvent. The organic solvent can be acetonitrile, ethanol, methanol, isopropanol, dimethyl sulfoxide (DMSO), N,N-dimethyl formamide, or dimethylacetamide. The silane can be a trimethoxy, dimethoxy, monomethoxy, triethoxy, diethoxy, monoethoxy, trichlori, dichloro, or monochlorosilane to react with the silica shell. In some embodiments, the silane can have an alkyl, carboxylic acid, protected carboxylic acid, amine, protected amine, activated amine (hydroxyamine, hydrazine, hydrazide, etc.) aldehyde, protected aldehyde, azido, NHS, ethoxy, maleimide, thiol, or dithiol functional group. In some embodiments, the silane can be reacted to the silica shell followed by a subsequent functionalization. In some embodiments, the subsequent functionalization can be a reaction to form any of the foregoing functional groups. In some embodiments, the functionalized silica shell can be reacted with a functional group present on an antibody or functionalized oligonucleotide. In some embodiments, the antibody functional group can be a thio, aldehyde, amine, or carboxylic acid. In some embodiments, the oligonucleotide functional group can be an azide, alkyne, aldehyde, amine, activated amine, carboxylic acid, aklynyl halide, or thiol. The functionalized oligonucleotide can be synthesized or purchased. In some embodiments, when the functionalized oligonucleotide is synthesized in situ, the synthesis can involve the selected functionalized nucleotides available from Glen Research (Sterling, Va.). In some embodiments, when the functionalized oligonucleotide is purchased, it can be purchased from IDT (San Diego, Calif.), Trilink (San Diego, Calif.), or Midland Oligos (Midland, Tex.).

In some embodiments, the attachment of the oligonucelotide or antibody to the functionalized nanoparticle can be accomplished by the bioconjugation methods described in Hermanson, G., Bioconjugate Techniques, Academic Press (1996), herein incorporated by reference in its entirety.

In some embodiments, when the nanoparticle species are functionalized with a biomarker binding moiety, e.g., an antibody or antibody fragment or other biomarker binding moiety that binds to one of the following: CD3, CD22, CD79a, Kappa, Lambda, Pax-5, ZAP-70, MPO, and TdT; the nanoparticle species can enter the cell and bind to its respective intracellular biomarker. The intracellular biomarker can be in the cytosol and/or nucleus, or on the nuclear membrane, or in or on another cellular compartment or structure. In some embodiments, the functionalized nanoparticles are small enough to enter the cell without disrupting the cell membrane. The cells can be treated with a permeabilizer so as to allow the functionalized nanoparticles to enter the cell without disrupting the cell membrane. In some embodiments, the permeabilizer can be a surfactant.

### Obtaining a Biomarker Signature of Each Observed Cell

In some embodiments, the biomarker signature can be obtained by counting the number or proportion of each of the functionalized nanoparticle species per cell. The number of cells or proportion of cells having identified normal or abnormal morphological profiles in the sample can be totaled, weighted, or otherwise determined. In some embodiments, the number of cells or proportion of cells having identified normal or abnormal morphological profiles in a sample can be stored in a HIPAA-compliant computer storage system and compared against a different sample from the same subject. In some embodiments, the different samples from the same subject can be obtained at different timepoints. In some embodiments, the different samples from the same subject can be obtained from different tissue types of the subject. The HIPAA-compliant computer system or storage system can be one which is specifically configured so as to comply with the United States Health Insurance Portability and Accountability Act (HIPAA) requirements for computer systems.

In some embodiments, a software program on a HIPAA-compliant computer system can be used in the method of detecting the biomarker-morphological profile of a cell. The step (d) of illuminating the nanoparticle-cell complexes with evanescent light and detecting the resonant light scattering from each observed complexed nanoparticle, to obtain a biomarker signature of each observed cell in the method of detecting the biomarker-morphological profile of a cell can further comprise:
(i) using a software program that counts the number of each of the functionalized nanoparticle species per cell and processes images in each cell in the field of view;
(ii) moving the field of view digitally;
(iii) using a software program to count the number of each of the functionalized nanoparticle species per imaged cell in the next field of view and repeating steps (ii) and
(iii) until the entire substrate area is analyzed;
(iv) digitally combining all images obtained to generate a single image covering the entire selected substrate area; and
(v) generating from the data obtained for the entire substrate area the number of each of the functionalized nanoparticle species per imaged cell, the biomarker signature, of each substrate-adhered cell.

In some embodiments the software program stores the positional information for each imaged and/or observed cell.

In some embodiments, the software on the HIPAA-compliant computer system can count the number of each of the functionalized species per cell and process images in each cell in the field of view. In some embodiments, the software can identify the nanoparticle by identifying the resonant light signature obtained from the nanoparticle. The software can identifty the circumference of the light signature, the color of the light signature, and reduce the bloom of the light signature, of each nanoparticle in the field of view. In some embodiments, the color of the light signature can be identified using spectral identification algorithms. In some embodiment, the software can identify one nanoparticle as a circular light source.

In some embodiments, the field of view is from about 0.25 μm² to about 2.5 cm². In some embodiments, the field of view can be from about 100 μm² to about 1000 mm². In some embodiments, the field of view is 5 microns by 5 microns. In some embodiments, the field of view is 100 mm by 100 mm. In some embodiments, the field of view is round. In some embodiments, the field of view is square-shaped. The sides of the square-shaped field of view can be from 0.25 microns up to 2.5 centimeters. The field of view can cover one cell, or a plurality of cells. In some embodiments, the field of view can cover the area of the entire slide. The field of view can be digitally moved to view a different field of view from a previous image. The movement can occur via electronic servo-controlled motors which control the sample stage upon which the substrate is located. The software on the HIPAA-compliant computer system can identify each field of view within the substrate. The software can then count the number of each of the functionalized nanoparticle species per cell in the next field of view and repeating steps (ii) and (iii) until the entire selected substrate area is analyzed. The substrate area selected can be the entire substract or a portion thereof. The software can combine the results of each field of view for the entire selected substrate area so as to obtain a biomarker signature of the sample.

In some embodiments, the morphological features of each substrate-adhered cell can be associated with the biomarker signature of the contacted cells to detect the biomarker-morphological profile of each cell. The association can be made by comparing the corresponding physical location of the cells identified in the morphological features analysis when imaging the optical contrast agent properties to the physical location of the nanoparticles around the same area. For example, a cell may be identified as being a cancerous cell in by its morphological features in a brightfield image, and the diagnosis can be confirmed by analyzing which biomarkers are present on or within the cell by measuring which biomarker-binding functionalized nanoparticle species are present at the same corresponding area during the darkfield imaging process. In some embodiments, measuring which biomarker-binding functionalized nanoparticle species are present at the same corresponding area during the darkfield imaging process comprises associating the color of the resonant light signature of the particular size of nanoparticle with which biomarker-binding moiety was functionalized to that size of nanoparticle. In some embodiments, the association is a color-to-biomarker association.

In some embodiments, a method for detecting the biomarker-morphological profile of a cell can comprise an order of steps where the cells are contacted with an optical contrast agent before contacting with a functionalized nanoparticle species. When the biomarker-morphological profile is detected for a sample adhered to a substrate, the substrate may be stored for future analysis or retesting. In some embodiments, the method for detecting the biomarker-morphological profile of a cell can comprise:
(a) providing a sample comprising cells from a subject;
(b) adhering the cells to a substrate;
(c) contacting the substrate-adhered cells with an optical contrast agent;
(d) imaging morphological features of the contacted cells;
(e) converting the optical contrast agent to a colorless form;
(f) contacting the cells with one or a plurality of functionalized nanoparticle species, each functionalized nanoparticle species comprising a biomarker-binding moiety to form nanoparticle-cell complexes;
(g) illuminating the nanoparticle-cell complexes with evanescent light and detecting the resonant light scattering from each observed complexed nanoparticle, to obtain a biomarker signature of each observed cell; and
(h) associating the morphological features of the contacted cells with the biomarker signature of each substrate-adhered cell to detect the biomarker-morphological profile of each cell.

In some embodiments, the step of illuminating the nanoparticle-cell complexes with evanescent light and detecting the resonant light scattering from each observed complexed nanoparticle can further comprise releasing a first set of functionalized nanoparticles, contacting the cells with a next plurality of functionalized nanoparticles, and illuminating the nanoparticle-cell complexes with evanescent light and detecting the resonant light scattering from each observed complexed next plurality of nanoparticles.

In some embodiments, the optical contrast agent can be a leuco dye or any of optical contrast agents described herein. The leuco dye can be methylene blue, methylene green, red leuco dye, crystal violet, phenolphthalein, or thymolphthalein. The leuco dye can be converted to a colorless form by the addition of one or more electrons to the dye or by any of the methods described herein. Electrons can be added to the dye via a reduction method. The reduction method can be effected by an electrochemical reduction, photoreduction, or reaction with a reducing agent. In some embodiments, the leuco dye can be converted to a colored form by the removal of one or more electrons from the dye by any of the methods described herein. One or more electrons can be removed from the dye by an oxidation method. The oxidation method can be effected by an electrochemical oxidation, photooxidation, or reaction with an oxidation agent, by the methods described herein.

### Iterative Interrogations of Biomarker-Binding Functionalized Nanoparticles

The cells can be analyzed with a series of functionalized nanoparticle species. In some embodiments, the series of functionalized nanoparticle species can be iterative interrogations of the cell with functionalized nanoparticle pluralities, where a first plurality of functionalized nanoparticles are first contacted with a cell, followed by illuminating and detecting the first plurality of functionalized nanoparticles on the cell, followed by removing the first plurality of functionalized nanoparticle from the cell, followed by contacting the cell with a second plurality of functionalized nanoparticles. In some embodiments, the method for detecting the biomarker-morphological profile of a cell can further comprise: (d)(2) removing a first plurality of functionalized nanoparticles; and (d)(3) contacting the cells with a second plurality of functionalized nanoparticle species. In some embodiments, the selection of the second plurality of functionalized nanoparticles can be dependent upon the results of which first plurality of functionalized nanoparticles were detected on the cell. For example, if a first plurality of functionalized nanoparticles are contacted with a cell and found to indicate the presence of a first biomarker which may be indicative of a particular cell condition or disease state, the second plurality of functionalized nanoparticles to be contacted with the cell can be functionalized with a second biomarker-binding moiety, which binds to a second biomarker which is confirmatory for cell condition or disease state. In some embodiments, there can be additional iterations of contacting the cell with a third plurality of functionalized nanoparticles, where the third biomarker-binding moiety which binds to a third biomarker can also be confirmatory of the disease or condition of the cell. In some embodiments, there can be from 1 to 50 iterations of successive interrogations of the cell with a next plurality of functionalized nanoparticles, where the next plurality of functionalized nanoparticles can be comprised of a nanoparticle functionalized with a biomarker-binding moiety which was different from the previous biomarker-binding moieties on the functionalized nanoparticles. In some embodiments, there can be, for example, 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 1 to 25, 1 to 30, 1 to 35, 1 to 40, 1 to 45, 1 to 50, 2 to 50, 3 to 50, 4 to 50, 5 to 50, 6 to 50, 7 to 50, 8 to 50, 9 to 50, 10 to 50, 11 to 50, 12 to 50, 13 to 50, 14 to 50, 15 to 50, 20 to 50, 30 to 50, 40 to 50, or any of the ranges of the foregoing, numbers of iterations of successive interrogations of the cell with a next plurality of functionalized nanoparticles.

In some embodiments, each species of functionalized nanoparticle species can be functionalized with a different DNA oligonucleotide releasing system.

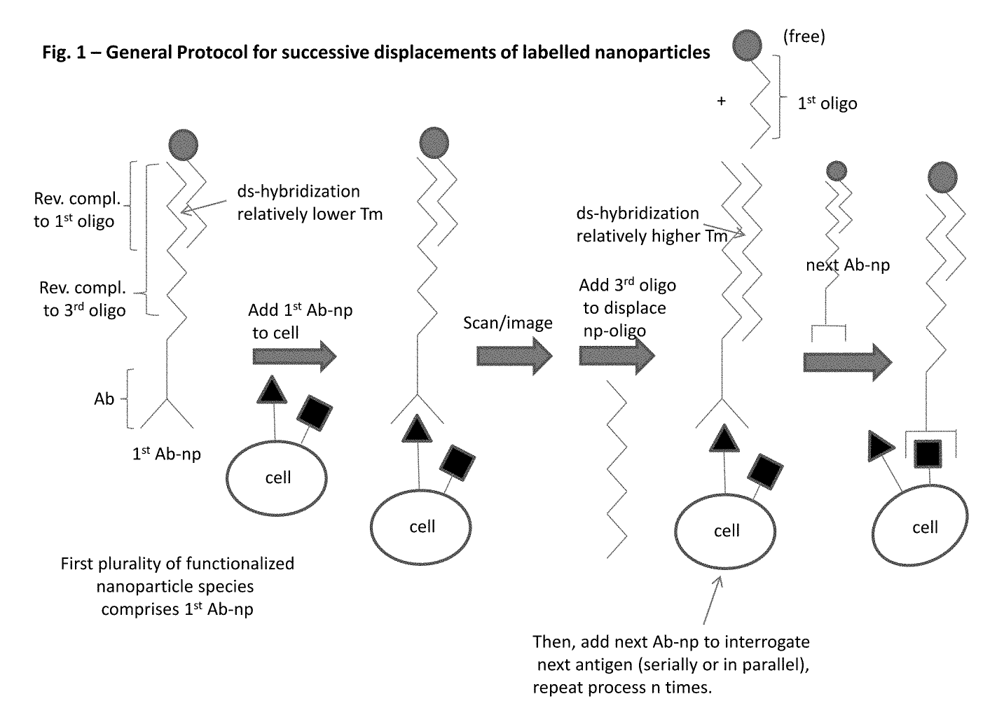

In some embodiments, the removal of a first plurality of functionalized nanoparticles can be achieved by displacing the first plurality of functionalized nanoparticles from the biomarker binding moieties. In some embodiments, the linker between each nanoparticle species in the first plurality of functionalized nanoparticles and its respective biomarker binding moiety comprises a first oligonucleotide bound to a first functionalized nanoparticle species and a second oligonucleotide bound to its respective biomarker binding moiety, where the second oligonucleotide comprises a portion complementary to at least a portion of the first oligonucleotide, and hybridization of the first oligonucleotide to the second oligonucleotide forms a linker comprising a double-stranded nucleic acid in these oligonucleotide-linker functionalized nanoparticle species. In some embodiments the first, second and third oligonucleotides may be the same for each of the functionalized nanoparticle species and respective biomarker binding moiety in the first plurality of nanoparticles. Each functionalized nanoparticle species can be displaced from its respective biomarker binding moiety by binding of a third oligonucleotide to the first oligonucleotide with the hybrid formed by hybridization of the third oligonucleotide and the first oligonucleotide exhibiting a melting temperature higher than the melting temperature of the double-stranded nucleic acid formed by hybridization of the first and second oligonucleotide, as shown in FIG. 1. In other embodiments, first, second and third oligonucleotides associated with each functionalized nanoparticle species and its respective biomarker binding moiety may be different for each nanoparticle species and its respective biomarker binding moiety. For example, in the first plurality of functionalized nanoparticles, the second functionalized nanoparticle species may comprise a fourth oligonucleotide, its respective biomarker binding moiety may comprise a fifth oligonucleotide, and the displacing oligonucleotide may be a sixth oligonucleotide.

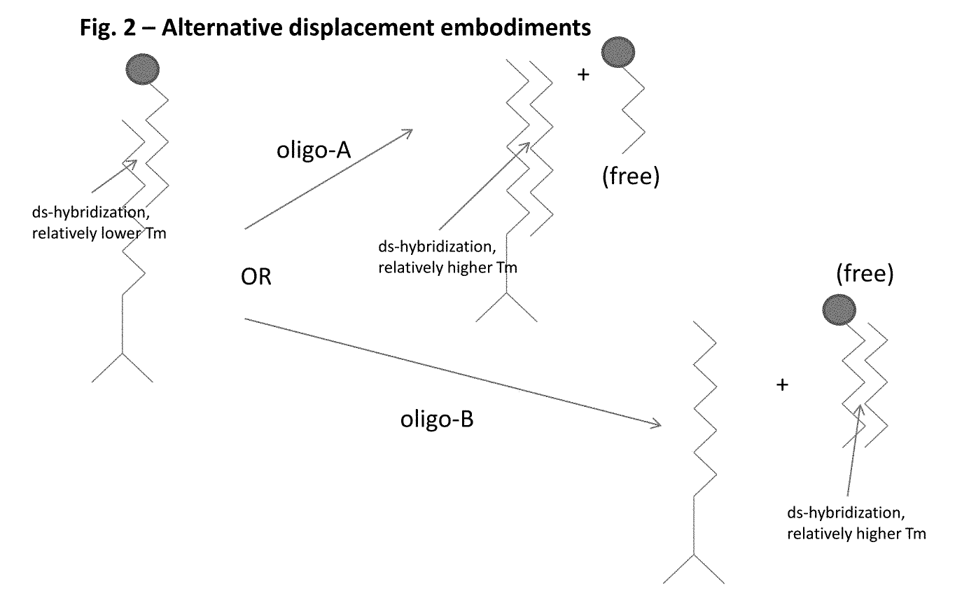

In some embodiments, the first plurality of functionalized nanoparticles comprises a first oligonucleotide and the biomarker binding moiety comprises a second oligonucleotide which is hybridized to the first oligonucleotide to form a duplex, as shown in FIG. 1, After scanning to detect the resonant light scattering of the first plurality of functionalized nanoparticles, the first plurality of functionalized nanoparticles can be displaced from the biomarker binding moieties by dissociating the duplex. In some embodiments of the invention, dissociation may be accomplished by heating the complexes above the melting temperature of the nucleic acid duplex. In some embodiments, the mixture comprising the complex can be warmed or the ionic strength reduced sufficiently to cause the hybridized duplex to dissociate. In some embodiments, a chemical or biological agent may be added to the complex to dissociate the duplexes. In some embodiments, a third competing oligonucleotide can be added in molar excess to disrupt the duplex of the first oligonucleotide and the second oligonucleotide. In this approach, an oligonucleotide hybrid is dissociated by competitive binding of one member of the hybrid pair to an excess of its complement. In some embodiments, the duplex can be disrupted by displacing either the first or second oligonucleotide to form a new duplex with either the first oligonucleotide or the second oligonucleotide, as shown in FIG. 2. “Displacing,” or “releasing” for the purpose of the present invention, may be accomplished by such methods as strand displacement or hydrolysis of the displaced strand catalyzed by a polymerase having a 3′ to 5′ or 5′ to 3′ exonuclease activity. In some embodiments, after displacing the first plurality of functionalized nanopanicle, the first plurality of functionalized nanoparticle can be washed away so as to eliminate the resonant light signal (RLS) from the first plurality of functionalized nanoparticles. The same cells can then be analyzed with a second biomarker-binding moiety conjugated to a functionalized nanoparticle with the same RLS signature as the first biomarker-binding moiety-nanoparticle properties. For example, after displacing and washing away a 25 nm Au nanoparticle functionalized with a first biomarker-binding moiety, a next 25 nm Au nanoparticle functionalized with a second biomarker-binding moiety can be contacted to the cells. In some embodiments, the cells can be stained and imaged after displacing the functionalized nanoparticles.

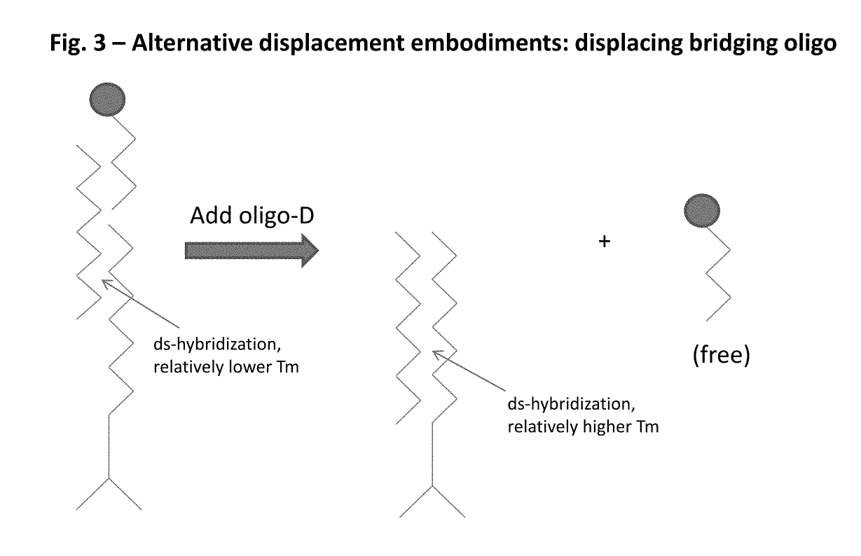

In some embodiments, the functionalized nanoparticle comprising a first oligonucleotide can be connected to the biomarker-binding moiety comprising a second oligonucleotide via an indirect hybridization with a third oligonucleotide, where a portion of the first oligonucleotide is complementary to a portion of the third oligonucleotide, and a portion of the second oligonucleotide is complementary to a different portion of the third oligonucleotide, as show in FIG. 3. The indirect hybridization duplex can be disrupted by displacing the third oligonucleotide from either the first or second oligonucleotides by adding a molar excess of a fourth oligonucleotide. In some embodiments, the fourth oligonucleotide can be to the second oligonucleotide with to form a stronger duplex (relatively higher Tm) than the duplex between the second oligonucleotide and the third oligonucleotide, as shown in FIG. 3. In some embodiments, a fifth oligonucleotide can be added in molar excess, optionally with the fourth oligonucleotide also present, where the fifth oligonucleotide can be complementary to the first or third oligonucleotides and form a stronger duplex between the fifth and first or third oligonucleotides thanthe first or third oligonucleotides with the second oligonucleotide. In some embodiments, the displaced functionalized nanoparticle can be removed. The removal can be effected by washing the cells with an aqueous solution.

In some embodiments, one or more iterations of interrogating biomarkers can be achieved by successive contacts with at least a second, third, up to ten or more plurality of functionalized nanoparticle species. In some embodiments, the one or more iterations of interrogating biomarkers can be, for example, one, two, three, four, five, six, seven, eight, nine, ten, fifteen, twenty, thirty, forty, or fifty or more times. In embodiments where oligonucleotide-linker functionalized nanoparticles are used, each plurality of functionalized nanoparticle species and respective biomarker binding moiety may comprise the same first, second third oligonucleotide for each oligonucleotide-linker functionalized nanoparticle species in a given plurality of oligonucleotide-linker functionalized functionalized nanoparticle species. Alternatively, each oligonucleotide-linker functionalized nanoparticle species and its respective biomarker binding moiety in each plurality of oligonucleotide-linker functionalized nanoparticle species may comprise a unique set of first, second and third oligonucleotides such that each biomarker binding moiety is associated with a unique set of first, second and third oligonucleotides. In this embodiment, from one, to ten or more successive rounds of displacement and contact with a new plurality of oligonucleotide-linker functionalized nanoparticle species can take place. Practitioners in the art will recognize that the ultimate limit to the number of iterations will be limited by the available cell area to contact a successive biomarker-binding moiety. The limit will be large, as a cell can range, for example, from 1 to 15 micrometers in diameter while a biomarker-binding moiety (e.g., antibody) is about 150 nanometers (0.15 micrometers) in diameter.

In some embodiments, after displacing the second or previous plurality of functionalized nanoparticles from the biomarker binding moieties, the following steps are performed:
(i) the biomarker-binding moieties of the functionalized nanoparticles which bound to the cell are associated with the biomarker binding moiety functionalized-nanoparticle is classified, and
(ii) the cells are contacted with a next plurality of nanoparticles functionalized with different biomarker binding moieties, and each nanoparticle species of the next plurality of nanoparticles are functionalized with different biomarker binding moieties that bind to a biomarker which is suspected of being associated with samples in which the first biomarker is present.

In some embodiments, the biomarkers targeted by the biomarker binding moieties in the second plurality of functionalized nanoparticle species each bind to a biomarker suspected of being associated with samples or conditions, diseases, or disorders that are also associated with the first biomarker. In this aspect, the methods of this disclosure are useful in detecting whether the associated biomarkers are present on the same or different cells, or populations of cells.

In some embodiments, the methods are useful in determining an association between the biomarkers that were bound during the first iteration and the biomarkers bound during the next or subsequent iterations. In some embodiments, the assocation can be made of the biomarkers bound during any iteration, and biomarkers bound during any other iteration. In some embodiments, the association can be made of all, or a portion, of the biomarkers bound to the cells in any iteration with biomarkers bound in a different iteration.

The association can be based on a systemic or tissue-based assay. The association can be a presumed biological correlation. In some embodiments, the method can determine whether two or more biomarkers which have been assumed to associate with the same cell are truly associated with the same cell or associated with different cells.

In some embodiments, the removing a first plurality of functionalized nanoparticles can be achieved by cleaving a linker between the nanoparticle and the biomarker-binding moiety. The linker can comprise a polynucleotide, modified polynucleotide, polyribonucleotide, modified polyribonucleotide, peptide, or glycan. The polynucleotide can comprise a DNA restriction enzyme sequence. The modified polynucleotide can comprise a di-thiol, diol, abasic, or uracil moiety within the polynucleotide sequence.

In some embodiments, the linker can comprise a peptide that further comprises a protease sequence. The protease sequence can be a trypsin or chymotrypsin protease recognition sequence. In some embodiments, the linker can comprise a glycan that further comprises an alpha-fucosidase recognition site. The alpha-fucosidase recognition site can be an alpha-1,2 fucoside bond. In some aspects, the linker can be cleaved with a peptidase, DNAase, and/or RNAse.

### Substrate Features

In some embodiments, the substrate can be comprised of glass silica, clear polymer (plastic), gold, or alumina. In some embodiments, the substrate can be ITO (indium tin-oxide). In some embodiments, the substrate can be FTO (fluoride tin-oxide). The substrate can be functionalized. The substrate functionalization can be patterned. The substrate functionalization can be a silane-linked cell biomarker, polymer-linked cell biomarker, silane-linked amine, silane-linked carboxylic acid, silane-linked biotin, polyfluorinated alkyl-linked amine, polyfluorinated alkyl-linked biotin, polymer-linked amine, polymer-linked carboxylic acid, polyethylene glycol (PEG), gold, polysaccharides (e.g., amine-functionalized dextran), teflon, fluorinated silane, silver, alumina, or glass silica. In some embodiments, the polysaccharides can be selected from: amino-functionalized dextran, amino-functionalized pullulan, amino-functionalized dextrin, and combinations thereof. In some embodiments, the substrate can comprise features to identify which region of the substrate is being imaged. The features can vary per region of the substrate so as to enable which region is being imaged. The features can comprise physical differences in the substrate at specific parts of the substrate. In some embodiments, the features can be: mirrors, lines, dots, particular shapes, barcodes, 2-D barcodes, or patterns or combinations thereof.

### Compositions/Combinations/Kits

A composition is described for the detection of a cellular biomarker signature. In some embodiments, the composition can comprise a plurality of functionalized nanoparticles where the nanoparticles are functionalized with a biomarker-binding moiety. In some embodiments, the functionalized nanoparticles can further comprise: a nanoparticle functionalized with a first oligonucleotide; a biomarker-binding moiety functionalized with a second oligonucleotide, and the first oligonucleotide is complementary to a portion of the second oligonucleotide, and the first and second oligonucleotide form a hybridized duplex. In an alternative embodiment, the functionalized nanoparticles can further comprise a nanoparticle functionalized with a first oligonucleotide; a biomarker-binding moiety functionalized with a second oligonucleotide; and a third oligonucleotide, where the first oligonucleotide is complementary to a portion of the third oligonucleotide, the second oligonucleotide is complementary to a separate portion of the third oligonucleotide, and the first and second oligonucleotides form a hybridized duplex to the third oligonucleotide.

In some embodiments, a combination or kit for the detection of a cellular biomarker signature can comprise a plurality of biomarker-binding moiety functionalized nanoparticle species. In some embodiments, the combination of functionalized nanoparticle species can further include: a nanoparticle species bound to a first oligonucleotide and a biomarker-binding moiety bound to a second oligonucleotide, where the first oligonucleotide is complementary to a portion of the second oligonucleotide, and the first and second oligonucleotide form a hybridized duplex. In some embodiments, the combination of functionalized nanoparticle species can further comprise: a nanoparticle functionalized with a first oligonucleotide; a biomarker-binding moiety functionalized with a second oligonucleotide; and a third oligonucleotide, where the first oligonucleotide is complementary to a portion of the third oligonucleotide, the second oligonucleotide is complementary to a separate portion of the third oligonucleotide, and the first and second oligonucleotides form a hybridized duplex to the third oligonucleotide. In some embodiments, the plurality of functionalized nanoparticle species can comprise a mixture. In some embodiments, the plurality of functionalized nanoparticle species can be segregated before use. In some embodiments, the plurality of functionalized nanoparticle species can be segregated, for example, into separate vessels and contacted separately with cells, or combined before contacting a mixture with cells.

In some embodiments, a combination or kit for the detection of a cellular morphological biomarker signature can comprise a plurality of functionalized nanoparticle species and an optical contrast agent. In some embodiments, the plurality of functionalized nanoparticle species can comprise a mixture. In some embodiments, the plurality of functionalized nanoparticle species can be segregated before use.

A combination or kit is one featured embodiment for the detection of a cellular morphological biomarker signature using iterations of pluralities of functionalized nanoparticles with biomarker-binding moieties, where each plurality of functionalized nanoparticle species with biomarker-binding moieties can be releasable by the methods described herein. Each plurality of functionalized nanoparticle species with biomarker-binding moieties can be segregated from the other pluralities of functionalized nanoparticle species. In successive iterations, the plurality of functionalized nanoparticle species can comprise the same plurality of nanoparticles, but functionalized with different biomarker-binding moieties than those in the functional nanoparticle species comprising a previous plurality of functionalized nanoparticle species. As a non-limiting example, a first plurality of functionalized nanoparticles can comprise a first functionalized nanoparticle species comprising a first nanoparticle with a first biomarker-binding moiety, a second plurality of functionalized nanoparticle species comprising a second nanoparticle with a second biomarker-binding moiety, and a third functionalized nanoparticle species comprising a third nanoparticle with a third biomarker-binding moiety. After contacting the first, second, and third species of functionalized nanoparticles with their respective biomarker-binding moieties to a cell and imaging the cell-functionalized nanoparticle complexes, the first, second, and third functionalized nanoparticles can be released from their respective biomarker-binding moieties. A next plurality of functionalized nanoparticles comprising a fourth functionalized nanoparticle species comprising the first nanoparticle and a fourth biomarker-binding moiety, a fifth functionalized nanoparticle species comprising the second nanoparticle and a fifth biomarker-binding moiety, and a sixth functionalized nanoparticle species comprising the third nanoparticle and a sixth biomarker-binding moiety can be contacted to the cell. In some embodiments, the first plurality of particles may comprise 3, 4, 5, 6, 7, 8, 9, 10, or any integer up to 50 functionalized nanoparticle species. Alternatively, in some embodiments, the fourth functionalized nanoparticle species comprising a fourth nanoparticle and a fourth biomarker-binding moiety, a fifth functionalized nanoparticle species comprising a fifth nanoparticle and a fifth biomarker-binding moiety, and a sixth functionalized nanoparticle species comprising a sixth nanoparticle and a sixth biomarker-binding moiety.

The combination or kit can comprise, pluralities of functionalized nanoparticle species, each plurality comprising, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more pluralities of functionalized nanoparticle species. A kit may comprise, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more pluralities of functionalized nanoparticle species. The number of pluralities of functionalized nanoparticles species in a kit will depend on the number of biomarkers to be detected and the number of biomarkers detected in each multiplex assay, and the number of replicates, controls, or duplicates in the assay design.

In some embodiments, the combination can comprise 2 to 5 functionalized nanoparticle species. In some embodiments, the next plurality of nanoparticles, functionalized with a different biomarker-binding moiety than used in a previous iteration. In some embodiments, a subsequent plurality of functionalized nanoparticle species may comprise a different plurality of nanoparticles, functionalized with the same or a different biomarker-binding moiety used in a previous plurality of functionalized nanoparticle species. This embodiments can be used, for example, to confirm the presence of a biomarker using a different functionalized nanoparticle species that may comprise the same or a different nanoparticle and/or the same or a different binding moiety for the same biomarker in a different plurality of functionalized nanoparticle species used in different steps. In some embodiments, the pluralities of functionalized nanoparticles can be segregated before use. In some embodiments each functionalized nanoparticle comprising each plurality of functionalized nanoparticle species may be combined in a mixture. Alternatively, each functionalized nanoparticle comprising each plurality of functionalized nanoparticle species may be segregated until use or added to cells one at a time or in submixtures.

As described herein, “segregated” means physically separate. Segregated components can be in separate containers or vessels, or in separate sections of a container, or separated by a seperatable medium, which can be removed to yield a mixture.

A composition is described for the detection of a cellular biomarker morphological profile, the composition comprising a plurality of functionalized nanoparticles and an optical contrast agent. The optical contrast agent can be the optical contrast agents described herein.

A “kit” for detecting the presence of an analyte in a sample by the methods of the invention may, by way of example, comprise at least one container means having disposed therein a functionalized nanoparticle specific for the selected analyte. The kit may further comprise other container means comprising one or more of the following: buffers, solutions or other reagents and materials necessary for performing biological morphological profiling of a cell; and buffers, solutions or other reagents and materials necessary for detecting the optical properties of an optical contrast agent contacted with a cell. Preferably, the kit further comprises instructions for use. The kit, if intended for diagnostic use, may also includes notification of a FDA approved use and instructions therefor,

A kit is described for the detection of a cellular biomarker signature. In some embodiments, the kit can comprise a plurality of functionalized nanoparticles, an optical contrast agent, and a mountant. The mountant can have a refractive index (RI) substantially the same, or within 0.1 RI to that of the fixed cells. In some embodiments, the mountant can have a RI of 1.52.

### Homogenous Assay

In some embodiments, the biomarker signature of a cell can be detected in a homogeneous assay, the assay comprising the steps:
(a) providing a sample comprising cells from a subject;
(b) contacting the cells with one or a plurality of functionalized nanoparticle species, each functionalized nanoparticle species comprising a biomarker-binding moiety, and forming nanoparticle-cell complexes through binding of the nanoparticle species comprising a biomarker-binding moiety to its respective biomarker;
(c) adhering the functionalized nanoparticle-cell complexes to a substrate;
(d) illuminating the nanoparticle-cell complexes with evanescent light and detecting the resonant light scattering from each observed complexed nanoparticle, to obtain a biomarker signature of each observed cell,
   where unbound functionalized nanoparticles are not removed from the field of view. In some embodiments, not removing the unbound functionalized nanoparticles from the field of view can be to not wash the field of view. Often, unbound species are washed from a target to reduce background noise. Eliminating the wash step had the advantage of a faster overall operation time. The functionalized nanoparticles are specific to the biomarker on the cell, and can substantially contact the cell such that little to none signal is observed for the unbound functionalized nanoparticles.

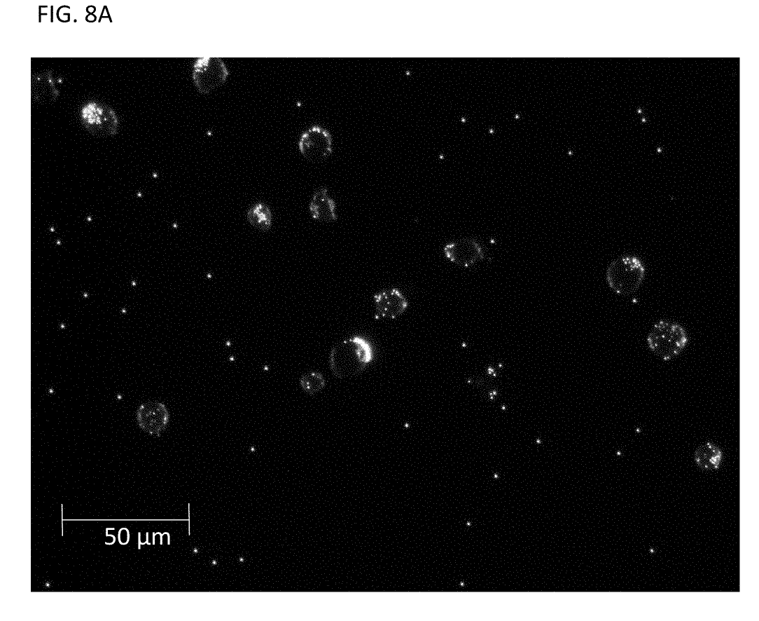

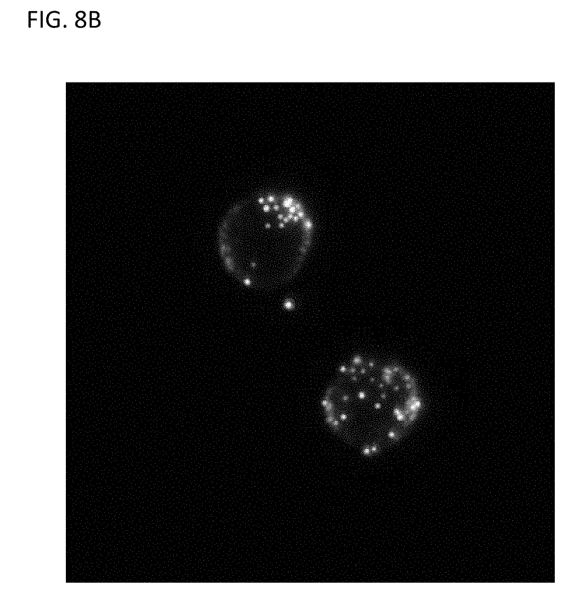

In some embodiments, the functionalized nanoparticles contacted to the cells can be used to identify the cellular features and morphology when the functionalized nanoparticle is functionalized with a biomarker-binding moiety that binds to the biomarkers in the interrogated morphological feature. FIG. 8b shows an expanded image of the functionalized nanoparticle-contacted cells. The cells were not washed to remove the functionalized nanoparticles which did not contact the cells. The cellular shape is clearly identifiable from the relative location of the functionalized nanoparticles. This method can be used to identify cells, cellular morphologies, and biomarker signatures from the image of the functionalized nanoparticles.

### Automated Robot System

In some embodiments, the sample handling and detection steps, including, without limitation, reagent contact and mixing steps, and application of external force, complex formation, and detection can be performed by an automated robot system. In some embodiments, applying the external force to the cells contacted with the functionalized biomarker binding moieties can be handled by an automated robot system. In some embodiments, for example, the steps of providing a sample comprising cells from a subject, contacting the cells with one or a plurality of functionalized nanoparticle species, and adhering the functionalized nanoparticle-cell complexes to a substrate, can be performed by the automated liquid handling robot system. In some embodiments, the step of contacting the adhered cells with an optical contrast agent can be performed by the automated liquid handling robot system. The automated liquid handling robot system can comprise a controller, a servo mechanism, fluid lines, and optionally solenoids. The automated liquid handling robot system can be programmed to deliver the reagents described herein at selected times, for selected durations, to deliver selected volumes of reagents. In some embodiments, the controller can be programmed using Labview software. In some embodiments, the automated liquid handling robot system can include or exclude, for example, one or a plurality of automatic pipettes, one or a plurality of automatic pipettes syringe pumps, a Hamilton Microlab NIMBUS 96 channel liquid handling robot, a Hamilton Microlab STAR liquid handling robot, a Hamilton VANTAGE liquid handling system, a Tecan Freedom EVO liquid handling system, a Tecan Fluent liquid handling system, a Beckman Biomek liquid handling system, a Beckman BioRAPTR FRD liquid handling system, a Perkin Elmer JANUS liquid handling system, a Hudson Robotics SOLO liquid handling system, a Hudson Rbootcs Micro10× liquid handling system, a QiaCube liquid robot system, an Aurora VERSA liquid handling system, or an Epppendorf epMotion liquid handling system.

In some embodiments, detecting cell-functionalized nanoparticle complexes and detecting morphological images can also be handled by an HIPPA compliant automated system with cell recognition software. In some embodiments, images of the cell-functionalized nanoparticle complexes and morphological images of the cells are obtained and stored in an electronic medium, for example in a HIPPA compliant system. In some embodiments the images are accessed by, or provided to a doctor or pathologist for review in the doctor's or pathologist's office.

In some embodiments, the methods of this invention are useful in obtaining images of cell-functionalized nanoparticle complexes under ambient conditions which do not require use of a darkroom, in contrast to fluorescent labeling systems. In some embodiments, the samples may be viewed on a microscope in a doctor's or pathologist's office.

### Examples

The instant disclosure and examples herein documents the features of the methods of this invention, including the detection of cell-functionalized nanoparticle complexes to detect a biomarker signature of a cell, and the integrated morphological biomarker signature of a cell. Operating procedures for the presently disclosed method of detecting the biomarker-morphological profile of a cell using resonance-light scattering are set forth in the following examples.

The following Examples have been included to provide guidance to one of ordinary skill in the art for practicing representative embodiments of the presently disclosed subject matter. In light of the present disclosure and the general level of one of skill in the art, those of skill can appreciate that the following Examples are intended to be exemplary only of the embodiments and features described throughout this application, and appreciated by those skilled in the art, and that numerous changes, modifications, and alterations can be employed without departing from the scope of the presently disclosed subject matter.

### Materials and Methods

### General.

All materials were purchased from the vendors indicated, with the part numbers indicated. Centrifugation steps were performed as units of 1 gravitational force (“xg”).

Sample Preparation

Settling solution at 6% Dextran/0.32M Potassium oxalate was prepared by mixing 0.6 mL 10% Dextran 500 (Sigma #31392) in PBS, 0.16 mL 2M Potassium oxalate (Sigma# P0963) in water, and 0.24 mL PBS. Whole blood sample in 5 mM EDTA was added to settling solution at a ratio of 4:1. After a ten minute incubation, the supernatant was removed and centrifuged at 500×g for five minutes. Supernatant was removed down to 10 μl (microliters) to give leukocyte rich fraction (LRF). Cell-functionalized nanoparticle complexes can be formed through settling or throught application of external force, as disclosed herein. Other compositions for contacting cells with functionalized nanoparticles, as set forth in this disclosure can be used.

### Example 1

### Cell Labeling for Simultaneous Detection of Morphology and Phenotyping

As a non-limiting example of detecting the biomarker-morphology profile of a cell, cells were labeled with α-CD4 (BD #555344) and α-CD8 (BD#555631) nanoparticles. Other combinations of antibodies described herein and nanoparticles described herein can be used to detect the cell biomarker-morphology profile.

Coating Particles with Antibody—

Particles were first concentrated by centrifuging 1.0 ml of 150 nm Au particles (Cytodiagnostics# G150-20) to 100 μl at 800×g for five minutes, and separately, one ml of 100 nm Ag particles (NanoComposix# ECP1095) to 100 ul at 1200×g for five minutes. Particles were resuspended by sonication and followed by an addition of 500 μl (microliters) of 5 mM Sodium Bicarbonate. Particles were concentrated again to 100 μl (microliters) by centrifugation and resuspended by sonication.

To the Au and Ag concentrated particles, 20 ul of α-CD4 and α-CD8 were added, respectively (separately), at approximately 0.1 mg/ml Ab for 10 OD particles. An OD of particles is the reported concentration (per mL) at the maximum absorbance wavelength for the particles. Each solution was vortexed and sonicated every 15 minutes for one hour. One percent BSA in water was added before a 30 minute incubation period. Particles were washed three times with 1% BSA/1% PBS. After washing, solutions were sonicated and vortexed to resuspend particles in final volume of 100 μl (microliters) 1% BSA/1% PBS.

To label the cells, particles were diluted 1:10 in 1% BSA/1% PBS. One μL (microliter) of Au and Ag particles were added 500 (microliters), approximately 10,000 cells, of CCRF-CEM cells (ATCC# CRM-CCL-119). The CEM cell line is a leukemia cell line. The solution was centrifuged for one minute at 500×g three times, followed by vortexing the cells for resuspension.

Labeled cells were spread by applying 2 μl (microliters) to a glass slide in an area of 1 cm² and dried for five minutes. To fix cells, the slide was soaked in Coplin Jar with 100% MeOH for five minutes. The slide was transferred to a tube containing diluted 1:20 Giemsa stain (Ricca Chemical #3250-16) in water for one minute. The slide was washed with water to remove excess stain and allowed to air dry.

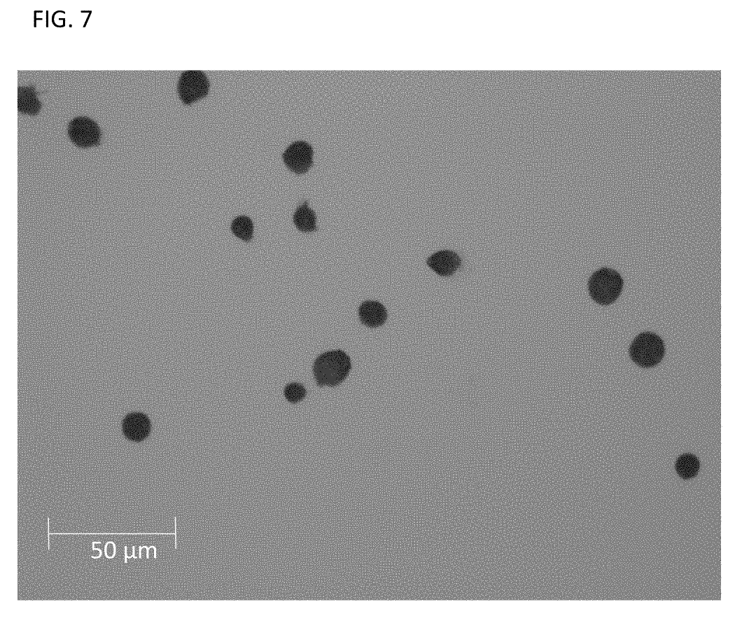

FIG. 7 shows stained cells were imaged for morphology detection in Bright-Field using a 20× objective, Olympus BX60M microscope and DP71 color camera.

FIG. 8 shows the same field imaged for phenotype detection using a 20× objective on Olympus BX60M microscope in Dark-field utilizing DarkLite Illuminator light source. Some of the functionalized nanoparticles can be observed which are not bound to the cells. In this exemplary homogeneous assay, the functionalized nanoparticles species which are introduced to the cells but do not bind to the cells have not been removed from the field of view.

### Example 2

### Cell Staining/Destaining

Labeled cells were spread by applying 2 μl of the cell suspension to a glass slide in an area of 1 cm² and dried for five minutes. To fix cells, the slide was soaked in a Coplin Jar with 100% MeOH for five minutes. The slide was transferred to a tube containing diluted 1:20 Giemsa stain (Ricca Chemical #3250-16) in water for one minute. The slide was washed with water to remove excess stain and allowed to air dry. Other stains, as disclosed herein may be used to stain cells.

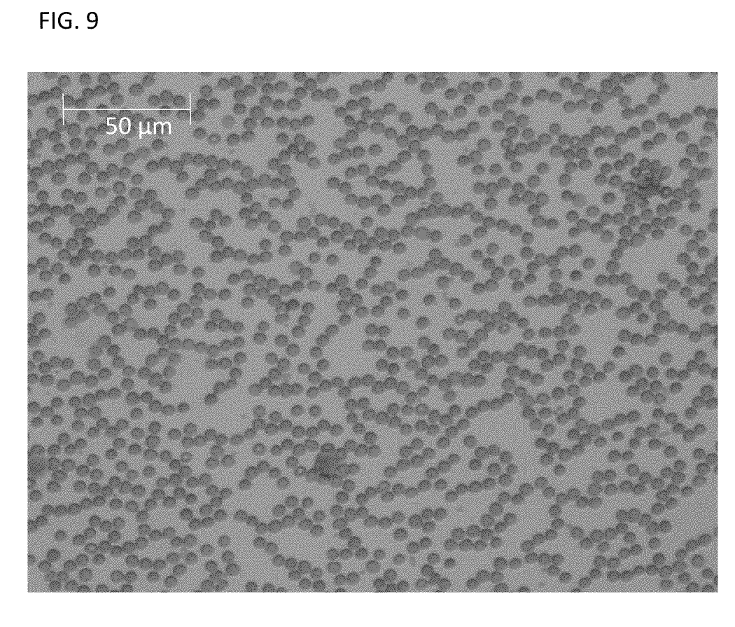

FIG. 9 shows an initial Brightfield image of Giemsa stained cells imaged for morphology detection in Bright-Field using 20× objective, Olympus BX60M microscope and DP71 color camera.

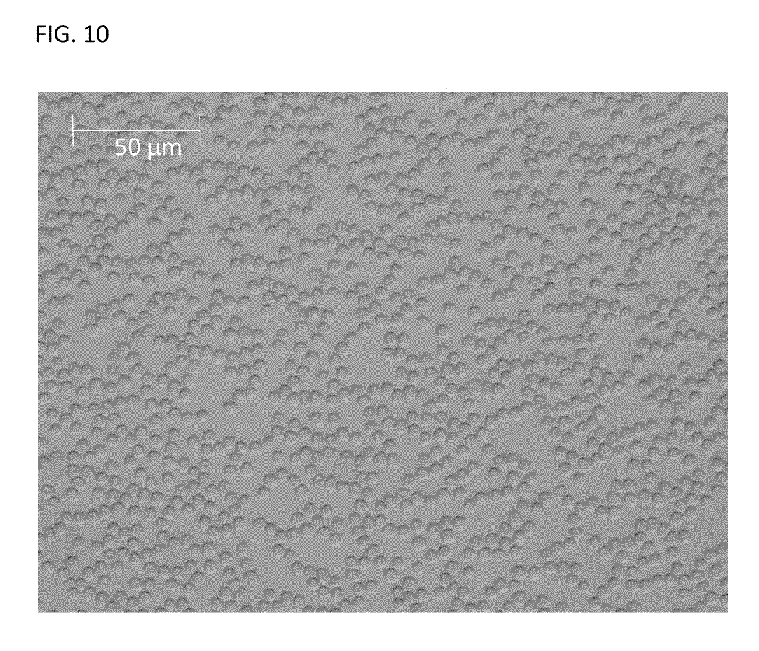

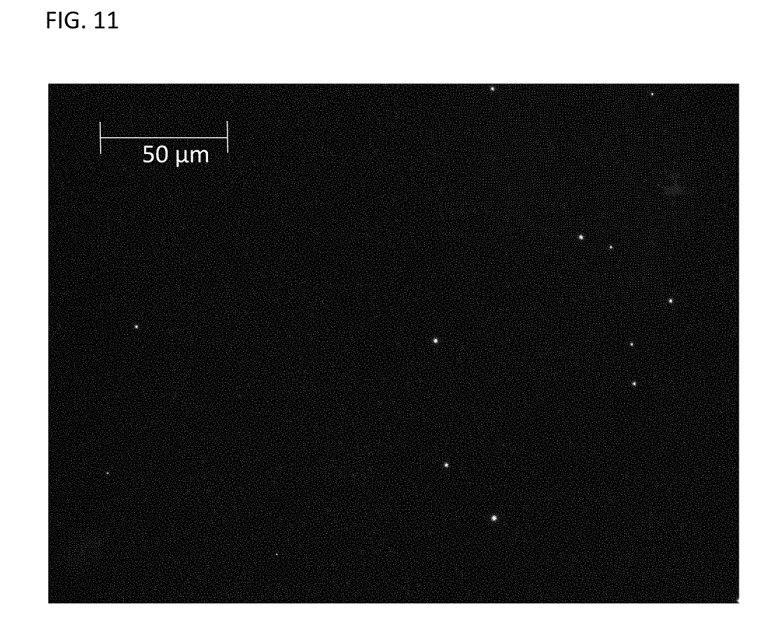

The cells were then destained as follows: Destain solution, pH 11.3, was prepared by adding 50 μl (microliters) of 100 mM sodium phosphate to one mL of 60% MeOH/40% Glycerol. 500 μl of destain was added to slide, incubated for 30 seconds, and washed with water. Before imaging, 411 DPX mountant (Sigma #06522) was applied to cell area followed by 18×18 mm cover glass. A mountant is any substance in which a specimen is suspended between a slide and a cover glass for microscopic examination. In some embodiments, the mountant can be comprised from a solution with about the similar refractive index of the cells. In some embodiments, the refractive index of the fixed cells is about 1.52. In some embodiments, the mountant can be immersion oil. FIG. 10 shows destained cells imaged in Bright-Field using 20× objective, Olympus BX60M microscope and DP71 color camera. The same field was imaged for residual Giemsa stain using 20× objective on Olympus BX60M microscope in Dark-field utilizing DarkLite Illuminator light source (FIG. 11).

### Example 3

### 3 Color Multiplexing

This example demonstrates the ability to detect functionalized nanoparticles comprising three different nanoparticles. To 0.2 mL of 1 OD particles, addition of 10 μl of 20 mM CTPEG mixtures (Nanocs# PG2-CATH-10k) was made to yield approximately 1 mM PEG. CTPEG mixtues are thiol carboxylic acid functionalized PEG, Molecular Weight of 10000. After 30 minute incubation at room temperature, 0.1% w/v Pluronic® F127 (BASF#51181981) was added and allowed to stand for an additional 30 minutes. Pluronic® block copolymers are synthetic copolymers of ethylene oxide and propylene oxide represented by the following chemical structure: HO(C2H4O)₁₀₁(C3H6O)₅₆(C2H4O)₁₀₁H. Particles were centrifuged at 3000×g for 10 minutes and resuspended in 200 μl mM MES, pH 6, 0.1% F127. Next, 10 mg of concentrated EDC (Thermo#77149) in distilled water was dissolved in one mL 5 mM MES, pH 6, 0.1 F127 to yield 52 mM. An addition of 11.5 μl (microliters) of 52 mM EDC solution was made to yield 2 mM EDC. Sulfo-NHS (Thermo#24520) at 2 mg was dissolved in 40 μl (microliters) of 5 mM MES, pH 6, 0.1% F127 to yield 230 mM Sulfo-NHS. Another addition of 6.5 μl (microliters) of 230 mM Sulfo-NHS was added to yield 5 mM NHS. After allowing it to react for five minutes, mixtures were spun at 3000×g for ten minutes, resuspended in 0.2 mL of 5 mM HEPES, pH 7.4, 0.1% F127 and anti-CD45 antibody (BD Biosciences#555480; 0.5 mg/ml) to final concentration of 100 μg/ml (microgram/milliliter) was added. Mixtures were reacted overnight at room temperature. One percent BSA was added and incubated for one hour. Then, particles were washed with 5 mM HEPES, pH 7.4, 0.1% F127, 0.1% BSA. Monodispersity was checked by imaging with 40× objective and DarkLite Illuminator light source.

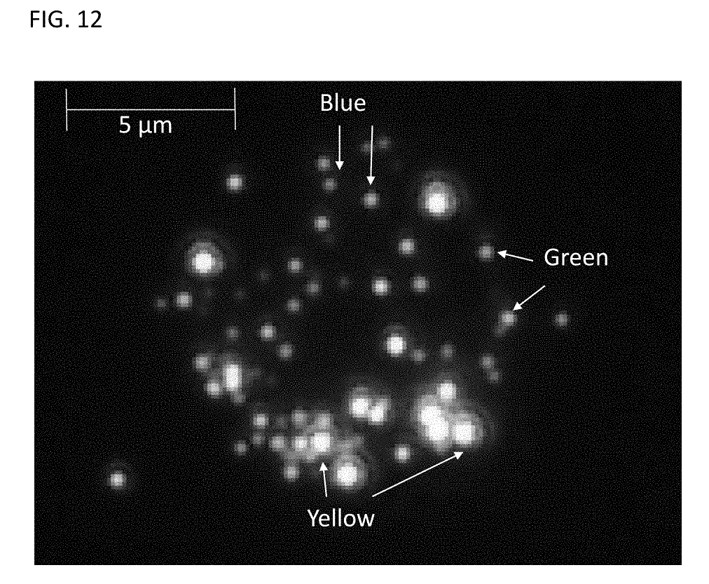

Anti-CD45 40 nm Ag particles (Nanocomposix# AGCN40-25M) were diluted 1:10 into 5 mM HEPES, pH 7.4, 0.1% F127, 0.1% BSA. Anti-CD45 50 nm Au particles (Nanocomposix# AUCN50-25M) and anti-CD45 80 nm Au particles (Nanocomposix# AUCN80-25M) were each diluted 1:20 into the same solution. To 10 μl (microliters) of concentrated 10× CCRF-CEM (ATCC# CRM-CCL-119) cell suspension of approximately 10,000 cells, 1 μl (microliter) of appropriate particle suspension was added. Centrifugation at 3000×g for one minute three times was performed. To a glass slide, 1 μl (microliter) of the cell-particle suspension per slide was applied to an approximate 1 cm² diameter area. The slide was then fixed for one minute in 100% MeOH soak in a Coplin jar. DPX mountant was applied at 5 μl (microliters) followed by a coverslip and imaged in Dark-field with DarkLite Illuminator light source and 40× objective (200 ms exposure), as shown in FIG. 12.

Any of the functionalized nanoparticles and or other features disclosed in this application can be used to multiplex detection of the biomarker signature and/or biomarker-morphological profile.

### Example 4

### Color Multiplexing

This example demonstrates the ability to detect functionalized nanoparticles comprising four different nanoparticles.

To 0.2 mL of 1 OD particles, addition of 10 μl of 20 mM CTPEG mixtures (Nanocs# PG2-CATH-10k) was made to yield approximately 1 mM PEG. After 30 minute incubation at room temperature, 0.1% w/v Pluronic® F127 (BASF#51181981) was added and allowed to stand for an additional 30 minutes. Particles were spun at 3000×g for 10 minutes and resuspended in 200 μl mM MES, pH 6, 0.1% F127. Ten mg of concentrated EDC (Thermo#77149) was dissolved in one mL 5 mM MES, pH 6, 0.1 F127 to yield 52 mM. An addition of 11.5 μl of 52 mM EDC was made to yield 2 mM EDC. Sulfo-NHS (Thermo#24520) at 2 mg was dissolved in 40 μl of 5 mM MES, pH 6, 0.1% F127 to yield 230 mM Sulfo-NHS. Another addition of 6.5 μl of 230 mM Sulfo-NHS was added to yield 5 mM NHS. After allowing it to react for five minutes, mixtures were spun at 3000×g for ten minutes, resuspended in 0.2 mL of 5 mM HEPES, pH 7.4, 0.1% F127 and anti-CD45 antibody (BD Biosciences#555480; 0.5 mg/ml (milligram/milliliter)) to final concentration of 100 μg/ml (microgram/milliliter). Mixtures were reacted overnight at room temperature. One percent BSA was added and incubated for one hour. Next, particles were washed with 5 mM HEPES, pH 7.4, 0.1% F127, 0.1% BSA. Monodispersity was checked by imaging with 40× objective and DarkLite Illuminator light source.

A multiplex cocktail containing yellow Anti-CD45 70 nm Au particles (Nanocomposix# AUCN70-25M), orange Anti-CD45 70 nm Au Nanourchins (Cytodiagnostics# GU70-20), green Anti-CD45 50 nm Au particles (Nanocomposix# AUCN50-25M), and blue Anti-CD45 50 nm Ag particles (Nanocomposix# AGCN50-25M) was prepared by mixing the particles at 1:2:2:4 parts, respectively, into 5 mM HEPES, pH 7.4, 0.1% F127, 0.1% BSA. To 10 μl of CCRF-CEM (ATCC# CRM-CCL-119) cell suspension of approximately 1,000 cells, 1 μl (microliter) of multiplex cocktail particle suspension was added. Centrifugation at 3000×g for one minute was performed.

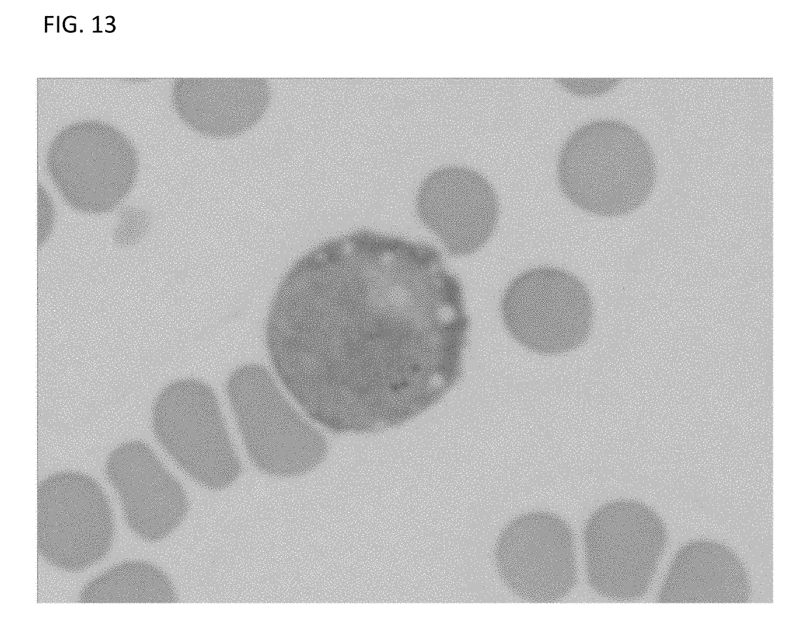

Labeled cells were then added to 200 μl (microliter) of a 1:2000 dilution of whole blood in 5% BSA/PBS, and washed three times with 200 μl (microliter) of 5% BSA/PBS at 100×g for 4 min. The entire sample was then applied to an assembled CytoFuge device containing a SuperFrost Plus slide (Fisher #12-550-15), and spun at 1500 rpm for 3 minutes to adhere cells to the slide using a Cytofuge 12 (Beckman-Coulter #X00-006082-001). The slide was then fixed for five minutes in 100% MeOH soak in a Coplin jar. Type HF Immersion oil (Cargille #16245) was applied at 7 μl (microliter) followed by a coverslip and imaged in Dark-field with DarkLite Illuminator light source and 40× objective (100 ms exposure) (image shown in FIG. 13).

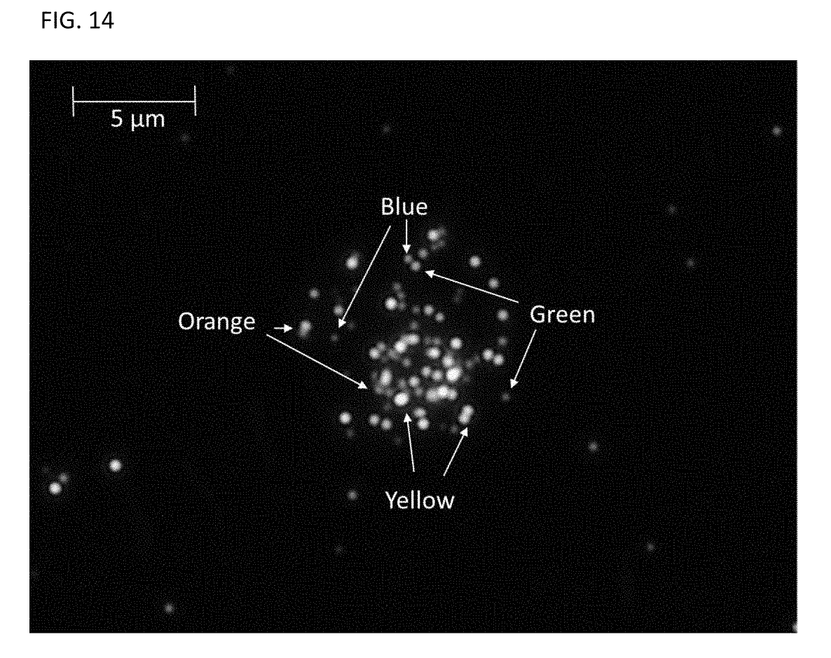

The coverslip and oil were removed by washing with propanol, and the slide was air dried. Next, 400 μl (microliter) of Giemsa stain (Ricca #3250-16) was applied to the cell area and incubated 4 minutes. Stain was removed with water wash. Next, 10 μl (microliter) of DPX Mountant (Sigma #06522) was applied with coverslip and the same cell area imaged in Brightfield using 40× objective (0.1 ms exposure) (image shown in FIG. 14).

Any of the functionalized nanoparticles and or other features disclosed in this application can be used to multiplex detection of the biomarker signature and/or biomarker-morphological profile. In accordance with the methods of this invention, it will be appreciated that 5, 6, 7, 8, 9 10, 11, 12, 13, 14, 15, or up to 50 different nanoparticles comprising functionalized nanoparticle species may be used in the multiplexed methods of this invention.

### Example 5

### Mountant Refractive Index Matching

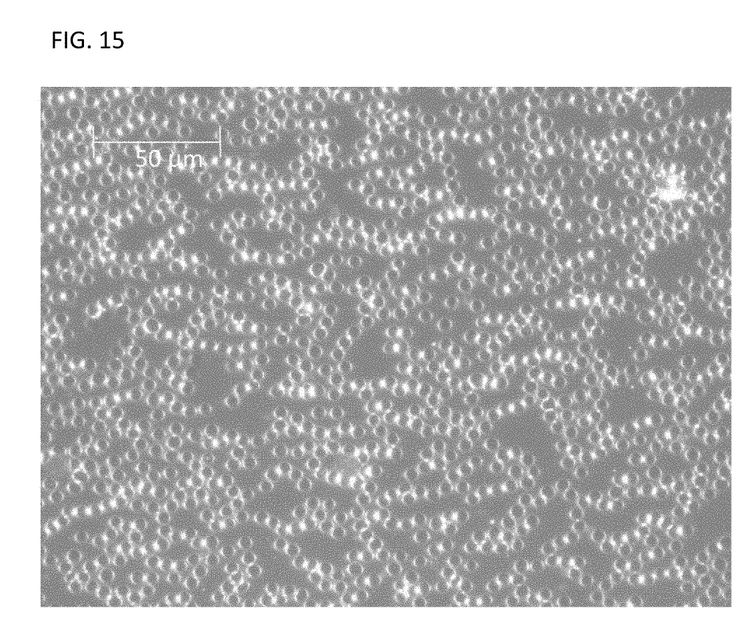

FIG. 15 shows an image of the CEM cells contacted with the functionalized nanoparticles as described in Example 4, with no mountant added. 20× Objective, 100 ms exposure, Darklite. The lack of a matching refractive index medium yields excessive white light scattering, thereby preventing imaging of the RLS signal of the functionalized nanoparticles.

### Example 6

### HIV Assessment Using Cellular Biomarker Signature

In some embodiments, the biomarkers identified on the cells can be used to identify the cell type and count of the cell type by adding all of the identified cell types exhibiting a particular biomarker. A cell from a subject's blood sample is contacted with a multiplex cocktail containing yellow anti-CD3 70 nm Au particles (Nanocomposix #AUCN70-25M), orange anti-CD-4 70 nm Au Nanourchins (Cytodiagnostics # GU70-20), and green anti-CD8 50 nm Au particles (Nanocomposix # AUCN50-25M). The cocktail is comprised of equal parts 1:1:1 of each of the functionalized nanoparticle pluralities. The particles are prepared in a buffer of 5 mM HEPES, pH 7.4, 0.1% F127, 0.1% BSA. To 10 ul of isolated cells from subject in a suspension adjusted to approximately 1000 cells, 1 microliter of the cocktail particle suspension is added. The solution is centrifuged at 3000×g for one minute to accelerate the cell-functionalized nanoparticle contacting. The cells are adhered to a slide and contacted with HF immersion oil as an RI matched mountant as described in Example 4.

The total cell count of cells exhibiting all of CD3, CD4, and CD8 are compared to the amount of cells which do not exhibit CD4. The amount of cells which are CD4 positive is compared against a threshold amount per volume of blood. If the amount of cells are below a threshold number, the subject is diagnosed with having HIV (Human Immunodeficiency Virus).

### Example 7

### Enhanced Labeling by Centrifugation

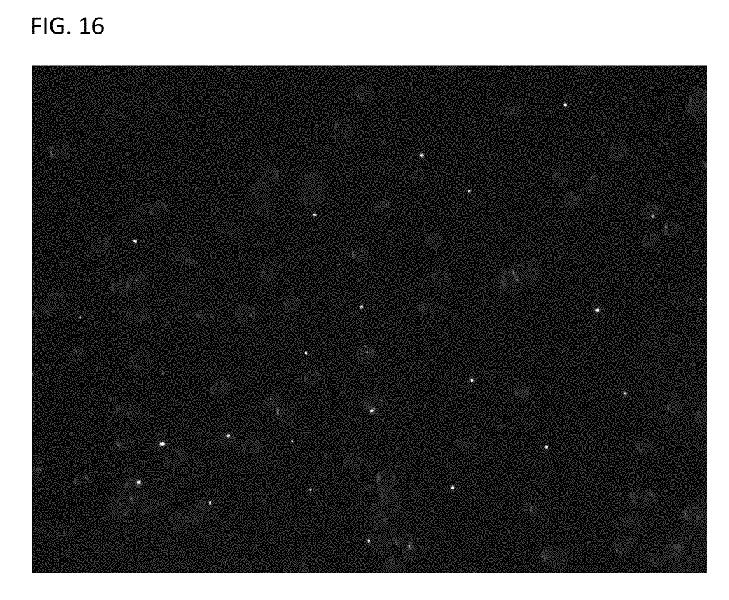

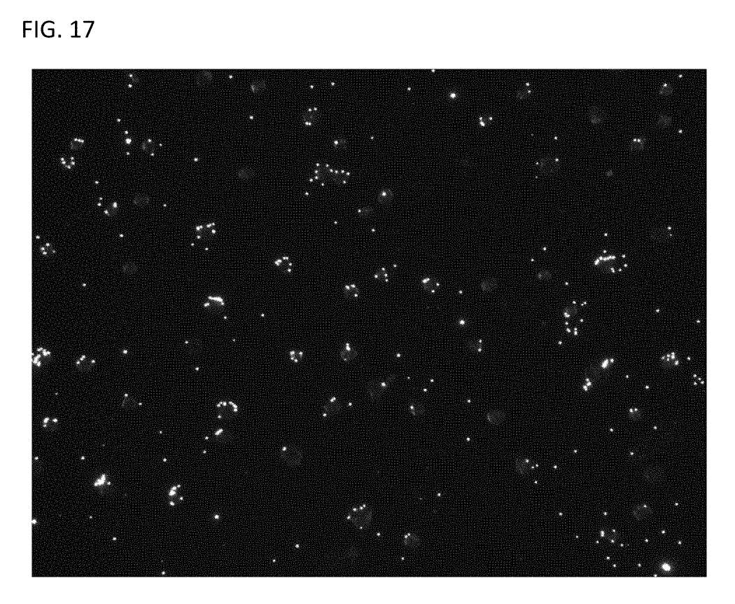

To determine the effects of subjecting cells contacted with functionalized nanoparticles to an external force, in order to increase the local concentration of the functionalized nanoparticles and cells, CCRF-CEM cells (ATCC) at 1×106 cells per mL were suspended in RPMI media (ATCC) supplemented with Fetal Cal Serum were reacted with biotinylated anti-CD45 antibody (BD Biosciences) at room temperature for 30 minutes. The cells were then washed via centrifugation at 500×g for 5 minutes with PBS+0.1% BSA five times. One microliter of cell suspension was applied to a microscope slide (SuperFrost Plus), allowed to air dry, then fixed in 100% MeOH for 1 minute and air dried. The slide was blocked with 10% BSA in PBS for 10 minutes at room temperature. The blocking solution was removed, and 70 nm Au nanoparticles functionalized with streptavidin were applied to the cell area. One slide was placed into a slide holder and centrifuged 2 minutes at 500×g in a swinging bucket centrifuge (centrifuge labeling). A second slide was incubated for 5 minutes at room temperature (passive labeling). Both slides were rinsed extensively with PBS, then water, and air dried. DPX mounting medium and coverglass was applied, and the cells were imaged using a dark-field microscope and color camera (Olympus). FIG. 16 shows the passive labelling slide (no centrifuge), and FIG. 17 shows the active labelling slide (with centrifugation). Significantly more streptavidin particles are bound to cells in the centrifuge labeling slide compared to the passive labeling slide, demonstrating that subjecting cells contacted with functionalized nanoparaticles to an external force that increases the local concentration of the functionalized nanoparticles results in enhanced labeling of the cells.

### Example 8

### Enhanced Labeling by Centrifugation of Blood Smear Example

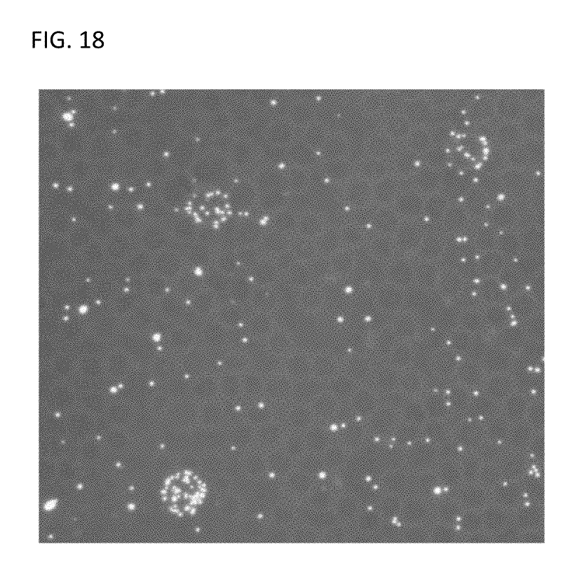

To demonstrate the effects of application of an external force on labeling of cells in a blood sample, a buffy coat from fresh EDTA treated blood was obtained, and a blood smear was prepared on a microscope slide using the wedge technique. After air drying, the blood smear was fixed in methanol for 10 minutes and allowed to dry. A silicone gasket was applied to create a reaction well to hold assay reagents. The slide was blocked with 10% BSA in PBS for 10 minutes at room temperature. The blocking solution was removed, and 50 μL of biotinylated anti-CD45 antibody (Becton Dickinson) was applied and incubated two hours at room temperature. The antibody solution was removed, the slide washed extensively with PBS-Tween-BSA buffer. 70 nm Au nanoparticles functionalized with streptavidin were applied and the slide was placed into a slide holder and centrifuged 2 minutes at 250×g in a swinging bucket centrifuge (centrifuge labeling). The slide was rinsed extensively with PBS-Tween-BSA buffer, then water, and air dried. DPX mounting medium and coverglass was applied, and the cells were imaged using a dark-field microscope and color camera (Olympus), as shown in FIG. 18. Lymphocytes and granulocytes, both known to express CD45 surface antigens, were labeled with Au functionalized nanoparticles while red blood cells were not.

### Example 9

### Multiplex Labeling of Blood Smear Example

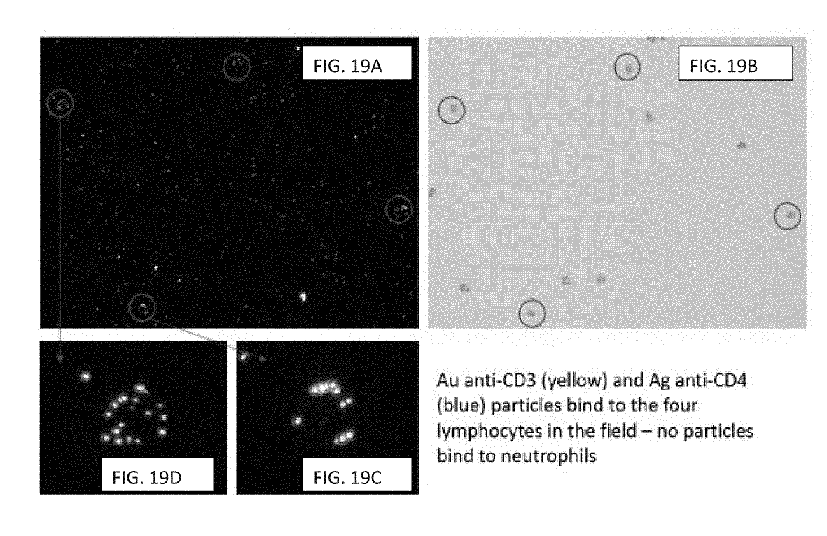

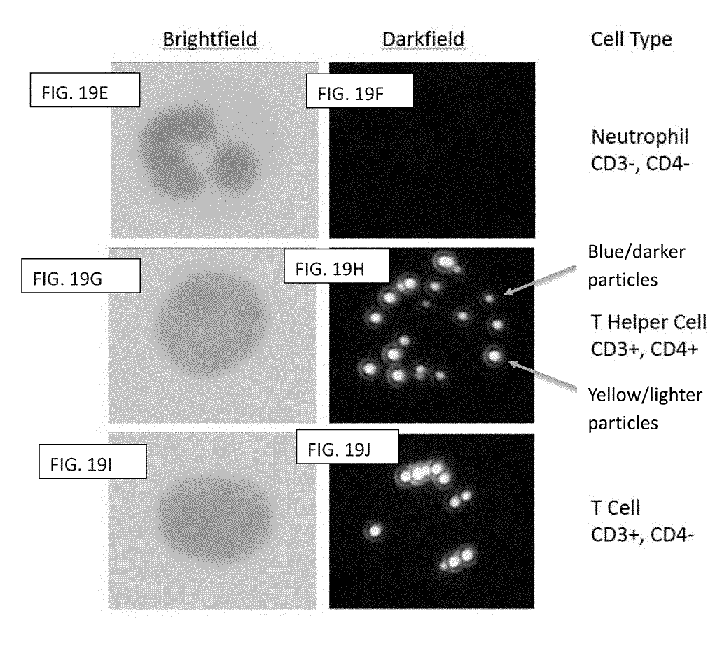

An exemplary method for multiplex labeling of cells in a sample was performed as described below. A Buffy coat from fresh EDTA blood was obtained from which a blood smear was prepared on a microscope slide using the wedge technique. After air drying, the blood smear was fixed in Neutral Buffered Formalin for 30 minutes, rinsed with water, and allowed to dry. A silicone gasket was applied to create a reaction well to hold assay reagents. The slide was blocked with 10% BSA in PBS for 10 minutes at room temperature. The blocking solution was removed and 0.1 OD 70 nm Au nanoparticles functionalized with anti-CD3 antibody and 0.1 OD 50 nm Ag particles functionalized with anti-CD4 antibody were applied. The slide was placed into a slide holder and centrifuged 1 minute at 500×g three times in a swinging bucket centrifuge (centrifuge labeling). The slide was rinsed extensively with PBS-Tween buffer, then water, and air dried. Index matching mounting medium and coverglass was applied, and the cells were imaged using a dark-field microscope and color camera (Olympus). The index matching medium was removed, and the slide was stained with Giemsa stain for 3 minutes, rinsed with water, and air dried. Mounting medium was applied, and the slide was imaged using a bright-field microscope and color camera (Olympus). White blood cells were first identified by their morphology in the stained, Brightfield image, as seen in FIGS. 19E-19J. Several Neutrophils, with their multi-lobed nucleus, were visible in the field of view, as seen in FIG. 19E. Lymphocytes, which lack the lobed nucleus in the Brightfield image, were further differentiated in their binding of anti-CD3 Au functionalized nanoparticles and anti-CD4 Ag functionalized nanoparticles that are visible in the Darkfield image, as seen in FIGS. 19G-19J. Several T cells, known to be CD3+, bound the anti-CD3 Au functionalized nanoparticles, as seen in FIG. 19H and FIG. 19J. As observed in FIG. 19H, one T helper cell, known to be both CD3+ and CD4+, bound both the anti-CD3 Au functionalized nanoparticles and the anti-CD4 Ag functionalized nanoparticles. One T cell, known to be CD3+ yet lack CD4, bound to the anti-CD3 Au functionalized nanoparticles but did not appreciably bind the anti-CD4 Ag functionalized nanoparticles. As observed in FIG. 19F, Neutrophils, which are known to lack CD3 and CD4 surface antigens, remained unlabeled with respect to functionalized particles.

### Example 10

### Assay of Whole Blood Cell Suspension

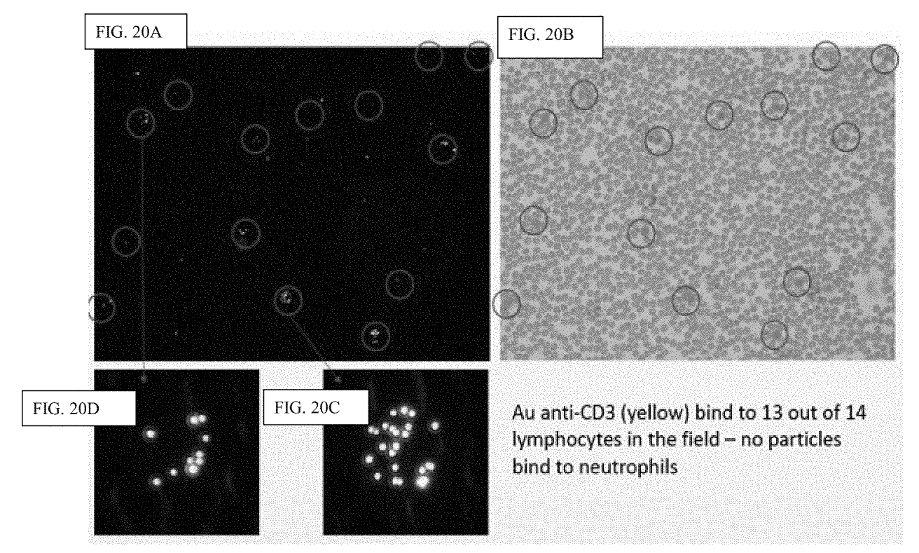

A Buffy coat from fresh EDTA blood was obtained, and cells were exchanged into PBS via centrifugation for 2 min at 500×g. 70 nm Au nanoparticles functionalized with anti-CD3 antibody was added and incubated 30 min with occasional mixing. The labeled cells were washed with twice with human plasma at low speed centrifugation (70×g) to remove unbound functionalized nanoparticles. Three microliters of the cell suspension was smeared on a Superfrost slide and air dried, then fixed for 5 min in 100% methanol. Index matching mounting medium and coverglass was applied, and the cells were imaged using a dark-field microscope and color camera (Olympus). The index matching medium was removed, and the slide was stained with Giemsa stain for 3 minutes, rinsed with water, and air dried. Mounting medium was applied, and the slide was imaged using a bright-field microscope and color camera (Olympus). In the field of view, 13 out of 14 lymphocytes showed bound Au functionalized nanoparticles. Neutrophils, which are known to lack CD3 surface antigens, remained unlabeled. FIG. 20A-D shows (clockwise from top left: A-D) Au anti-CD3 functionalized nanoparticles (yellow/lighter colors) bind to 13 out of 14 lymphocytes in the field. No functionalized nanoparticles were observed to bind to neutrophils.

### Example 11

### Electrically Enhanced Labeling

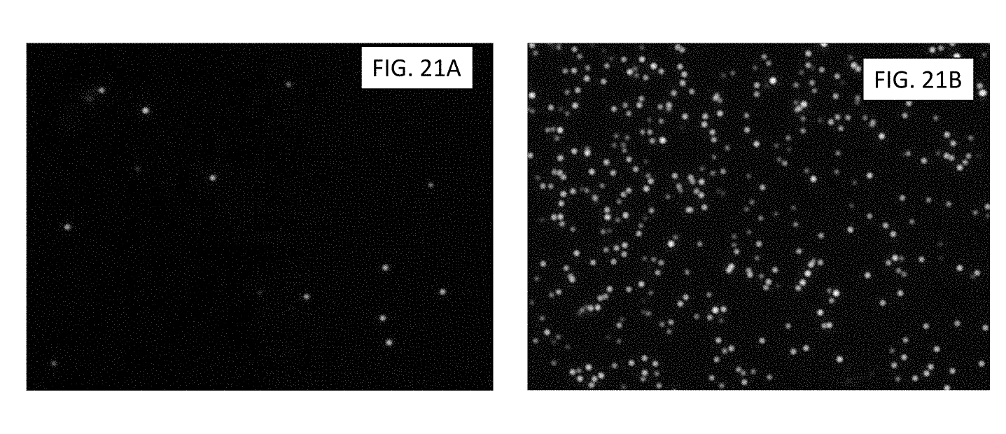

A BSA-biotin solution at a concentration of 5 micrograms/mL in a solution of free BSA at a concentration of 5 milligrams/mL in 10 mM MES buffer was prepared. The BSA-biotin/BSA solution was coated onto conductive, Indium-Tin-Oxide (ITO) glass slides (Nanocs) by immersing the slide into the solution for 30 minutes, then allowing the slide to air dry. In some embodiments, the conductive slide can be ITO. In some embodiments, the conductive slide can be Fluorine doped tin-oxide (e.g., TEC Glass™ materials from Pilkington (NJ, USA)). The free BSA and the BSA on the BSA-biotin adhered to the ITO surface, and the free BSA prevented biotin saturation at the surface. A 500 micron thick silicone gasket (Grace Bio-Labs, Inc.) was placed around the BSA-biotin area, and the slides were blocked with 1% Casein in PBS for 10 minutes. After removal of the blocking solution, 50 microliters of 0.1 OD, 70 nm Au nanoparticles functionalized with streptavidin was applied. One slide was incubated passively for 2 minutes while a second slide was covered with an additional conductive slide so that the conductive surfaces of the slides faced one another. Alligator clips from a power supply (BK Precision) were attached to the two conductive slides facing one another, and a voltage of 400 mV was applied for 2 minutes. At the end of two minutes, both the passively incubated and electrically enhanced slides were washed with TBS/0.05% Tween-20, water, and air dried. Imaging oil and coverglass were applied, and the BSA-biotin coated regions on each slide were imaged using a dark-field microscope and color camera (Olympus). While the passively incubated slide as shown in FIG. 21A has very few particles attached to its surface, the electrically enhanced slide is covered heavily with particles as shown in FIG. 21B.

### Example 12

### Multiplex Labeling of FFPE Tissue

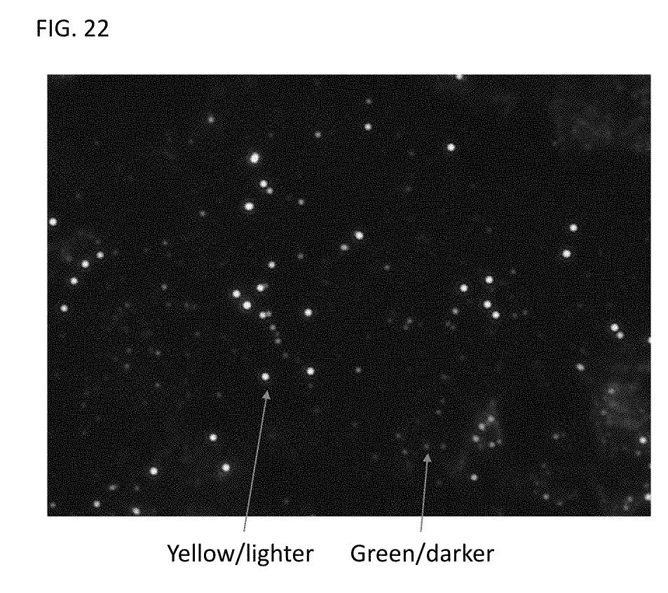

Fixed Formalin, Paraffin Embedded tissue arrays (Biomax-US) were depariffinized according to the following procedure: the slides were heated to 60° C. for 30 minutes, soaked in xylene 10 minutes, soaked in a fresh xylene wash for another 10 minutes, soaked in 100% ethanol for 5 minutes, soaked in 95% ethanol/water for 5 minutes, soaked in 70% ethanol/water for 5 minutes, then soaked in water for 5 minutes. A 500 micron thick silicone gasket (Grace Bio-Labs) was placed around the tissue array, and 50 nm (green) and 70 nm (Yellow) Au functionalized nanoparticles, passively coated with BSA, were applied to the tissue array via centrifugation at 1000×g for 3 minutes. After washing with TBS/0.05% Tween-20 and water, the slide was air dried. An index matching mountant and coverglass was applied, and the slide was imaged using a dark-field microscope and color camera (Olympus). Both colors of functionalized nanoparticles, the green 50 nm Au and yellow 70 nm Au, were clearly visible with distinguishable colors in the tissue sample, as shown in FIG. 22.

The inventions described and claimed herein have many attributes and embodiments including, but not limited to, those set forth or described or referenced in this Detailed Description. It is not intended to be all-inclusive and the inventions described and claimed herein are not limited to or by the features or embodiments identified in this Detailed Description, which is included for purposes of illustration only and not restriction.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for detecting the biomarker-morphological profile of a cell, the method comprising:
(a) providing a sample comprising cells from a subject;
(b) contacting the cells with one or a plurality of functionalized nanoparticle species, each functionalized nanoparticle species comprising a biomarker-binding moiety, and forming nanoparticle-cell complexes through binding of the functionalized nanoparticle species comprising a biomarker-binding moiety to its respective biomarker;
(c) adhering the functionalized nanoparticle-cell complexes to a substrate;
(d) illuminating the functionalized nanoparticle-cell complexes with epi-illumination or evanescent light and detecting the resonant light scattering from each observed complexed functionalized nanoparticle, to obtain a biomarker signature of each imaged cell;
(e) contacting the substrate-adhered cells with an optical contrast agent;
(f) imaging morphological features of the contacted cells; and
(g) associating the morphological features of the contacted cells with the biomarker signature of each substrate-adhered cell to detect the biomarker-morphological profile of each cell.

2. The method of claim 1, wherein the biomarker is present on the cell surface.

3. The method of claim 1, wherein the biomarker is present within the cell.

4. The method of claim 1, wherein the biomarker is selected from the following: CD1, CD2, CD3, CD4, CD5, CD6, CD7, CD8, CD9, CD10, CD11a, CD11b, CD11c, CD13, CD14, CD15, CD16, CD19, CD20, CD21, CD22, CD23, CD25, CD30, CD33, CD34, CD38, CD41, C43, CD45, CD56, CD57, CD58, CD61, CD64, C71, CD79a, CD99, CD103, CD117, CD123, CD138, CD138, CD163, CD235a, HLA-DR, Kappa, Lambda, Pax-5, BCL-2, Ki-67, ZAP-70, MPO, TdT, and FMC-7.

5. The method of claim 1, wherein the optical contrast agent is a leuco dye.

6. The method of claim 5, wherein the leuco dye is red leuco dye, methylene blue, crystal violet, phenolphthalein, thymolphthalein, or methylene green.

7. The method of claim 1, wherein the optical contrast agent is a cell stain selected from: Giemsa stain, Wright stain, Wright-Giemsa stain, May-Grünwald stain, Mallory trichrome, Periodic acid-Schiff reaction stain, Weigert's elastic stain, Heidenhain's AZAN trichrome stain, Orcein stain, Masson's trichrome, Alcian blue stain, May-Grünwald-Giemsa, van Gieson stain, Hansel stain, Reticulin Stain, Gram stain, Bielschowsky stain, Ferritin stain, Fontana-Masson stain, Hales colloidal iron stain, Pentachrome stain, Azan stain, Luxol fast blue stain, Golgi's method (reduced silver), reduced gold, Chrome alum/haemotoxylin stain, Isamin blue stain, Argentaffin stains, Warthin-Starry silver stain, Nissl stain, Sudan Black and osmium stain, osmium tetroxide stain, hematoxylin stain, Uranyl acetate stain, lead citrate stain, Carmine stain, safranin stain, and Ziehl-Neelsen stain.

8. The method of claim 1, wherein the optical contrast agent is a dye or colorant selected from: eosin Y, eosin B, azure B, pyronin G, malachite green, toluidine blue, copper phthalocyanin, alcian blue, auramine-rhodamine, acid fuschin, aniline blue, orange G, acid fuschin, neutral red, Sudan Black B, acridine orange, Oil Red O, Congo Red, Fast green FCF, Perls Prussian blue reaction, nuclear fast red, alkaline erythrocin B, and naphthalene black.

9. The method of claim 1, wherein the cells contacted with one or a plurality of functionalized nanoparticle species are subjected to an external force to increase the local concentration of the functionalized nanoparticles and cells.

10. The method of claim 9, wherein the external force is a gravitational, electric, or magnetic force.

11. The method of claim 10, wherein the gravitational force is generated by centrifugation.

12. The method of claim 10, wherein the magnetic force is effected by paramagnetic nanoparticles, wherein the core of the nanoparticle comprises a paramagnetic region and the shell of the nanoparticle comprises Ag, Au, Pt, Pd, Rh, Ro, Al, Cu, Ru, Cr, Cd, Zn, Si, Se, SiO₂, or mixtures or alloys thereof.

13. The method of claim 9, wherein charged polymers are added to the cells after step (a).

14. The method of claim 1, wherein imaging morphological features of the contacted cells comprises measuring an optical property of the optical contrast agent.

15. The method of claim 14, wherein an optical property of the optical contrast agent is selected from the following: absorbance, scattering, fluorescence, photoluminesence, Raman emission, and photoluminescent lifetime.

16. The method of claim 15, further comprising measuring an optical property of the optical contrast agent under a light field illumination with a microscope.

17. The method of claim 1, wherein the illuminating the functionalized nanoparticle-cell complexes with evanescent light and detecting the resonant light scattering from each observed functionalized complexed nanoparticle is done under a dark field illumination with a microscope.

18. The method of claim 17, further comprising using an illuminated slide holder to replace the darkfield condenser in the microscope.

19. The method of claim 18, further the illuminated slide holder uses total internal reflection to illuminate the slide holder.

20. The method of claim 18, wherein the illuminated slide holder comprises optical fibers to deliver light to the edge to the slide.

21. The method of claim 1, wherein the nanoparticles are from 10 to 200 nm in diameter.

22. The method of claim 1, wherein the nanoparticles are comprised of Ag, Au, Pt, Pd, Rh, Ro, Al, Cu, Ru, Cr, Cd, Zn, Si, Se or mixtures or alloys thereof.

23. The method of claim 22, wherein the alloy is an alloy of Au and Ag.

24. The method of claim 22, wherein the nanoparticles comprising mixtures of the listed metals further comprises discrete shells or layers.

25. The method of claim 22, wherein the nanoparticles are spherical, tubular, cylindrical, pyramidal, cubic, egg-shaped, t-bone-shaped, urchin- or rose-like (with spiky uneven surfaces) or hollow shaped.

26. The method of claim 22, wherein the nanoparticles comprising Si have a Si or SiO₂ shell.

27. The method of claim 22, wherein the nanoparticles comprising Si have a Au core.

28. The method of claim 1, wherein the biomarker-binding moiety is selected from the following: an antibody or fragment thereof, nanobody, DNA aptamer, DNA oligonucleotide, RNA aptamer, PNA aptamer, peptide aptamer, LNA aptamer, carbohydrate, and a lectin.

29. The method of claim 1, wherein the plurality of functionalized nanoparticle species is from 2 to 50 different species of functionalized nanoparticle species.

30. The method of claim 29, wherein each species of functionalized nanoparticle species is functionalized with a different species of biomarker-binding moiety.

31. The method of claim 30, wherein each species of functionalized nanoparticle species is functionalized with a different antibody.

32. The method of claim 31, wherein the antibody is a monoclonal or polyclonal antibody, or fragment thereof, or ScFv, or single-domain antibody (nanobody).

33. The method of claim 32, wherein the monoclonal antibody is an antibody to a cell-surface expressing protein, protein fragment, protein glycosylation pattern, or protein carbohydrate.

34. The method of claim 33, wherein the monoclonal antibody is selected from an antibody that binds to: CD1, CD2, CD3, CD4, CD5, CD6, CD7, CD8, CD9, CD10, CD11a, CD11b, CD11c, CD13, CD14, CD15, CD16, CD19, CD20, CD21, CD22, CD23, CD24, CD25, CD30, CD33, CD34, CD38, CD41, CD43, CD44, CD45, CD56, CD57, CD58, CD61, CD64, C71, CD79a, CD99, CD103, CD117, CD123, CD138, CD138, CD163, CD235a, Her-2, HLA-DR, Kappa, Lambda, Pax-5, BCL-2, Ki-67, ZAP-70, MPO, TdT, and FMC-7.

35. The method of claim 30, when the functionalized nanoparticle species is functionalized with an antibody selected from the following an antibody that binds to one of the following: CD3, CD22, CD79a, Kappa, Lambda, Pax-5, ZAP-70, MPO, and TdT; the functionalized nanoparticle species enters the cell and binds to their respective intracellular biomarkers.

36. The method of claim 35, wherein the intracellular biomarkers are in the cytosol and/or nucleus, or on the nuclear membrane.

37. The method of claim 35, wherein the functionalized nanoparticles are small enough to enter the cell without disrupting the cell membrane.

38. The method of claim 35, wherein the functionalized nanoparticles are smaller than 16 nm.

39. The method of claim 30, wherein each species of functionalized nanoparticle species is functionalized with a different DNA oligonucleotide.

40. The method of claim 1, wherein the morphological features comprise the cell surface shape, the cell nucleus shape, the chromatin shape, the nucleolar shape, the number of nucleolus, or combinations of the foregoing.

41. The method of claim 40, further comprising:
(h) diagnosing the subject's condition based on the biomarker-morphological profile of each cell.

42. The method of claim 41, wherein the subject's condition is having a hematological cancer, non-malignant hematological disorder, solid tumor, kidney disease, bladder disease, liver disease, or infectious disease.

43. The method of claim 42, wherein the hematological cancer is selected from: leukemia, lymphoma, and multiple myeloma.

44. The method of claim 42, wherein the non-malignant hematological disorder is selected from: anemia and sickle cell disease.

45. The method of claim 42, wherein the solid tumor is selected from: breast cancer, lung cancer, prostate cancer, colorectal cancer, and bladder cancer.

46. The method of claim 45, wherein the solid tumor is breast cancer and the biomarker is Her2.

47. The method of claim 42, wherein the kidney disease is selected from: acute kidney injury, chronic kidney disease, lupus nephritis, kidney rejection, and preeclampsia.

48. The method of claim 42, wherein the infectious disease is selected from: HIV, hepatitis, sexually transmitted diseases, and sepsis.

49. The method of claim 42, wherein the hematological cancer further comprises circulating cancer cells.

50. The method of claim 42, wherein the subject's condition is further identified by the lineage of the malignancy.

51. The method of claim 50, wherein the lineage of the malignancy is negative, Myeloid line, Lymphoid T cell line, or Lymphoid B cell line.

52. The method of claim 1, wherein the cells are white blood cells.

53. The method of claim 1, wherein at least 50% of the red blood cells are removed before contacting the cells with plurality of functionalized nanoparticle species.

54. The method of claim 1, wherein obtaining the biomarker signature further comprises counting the number or proportion of each of the functionalized nanoparticle species per cell.

55. The method of claim 54, wherein the number or proportion of cells having identified normal or abnormal morphological profiles in the sample are totaled.

56. The method of claim 1, wherein the adhering the functionalized nanoparticle cell complexes to a substrate further comprises adding a mountant.

57. The method of claim 56, wherein the volume of the mountant is 2 microliters.

58. The method of claim 55, where detecting the resonant light scattering from each observed complexed functionalized nanoparticle in step (d) further comprises:
(i) using a software program that counts the number of each of the functionalized nanoparticle species per cell and processes images in each cell in the field of view;
(ii) moving the field of view digitally;
(iii) using a software program to count the number of each of the functionalized nanoparticle species per cell in the next field of view and repeating steps (ii) and (iii) until the entire substrate area is analyzed;
(iv) digitally combining all images obtained to generate a single image covering the entire substrate area; and
(v) generating from the data obtained for the entire substrate area the biomarker signature of each substrate-adhered cell.

59. The method of claim 58, wherein the field of view is from 5 microns by 5 microns to 100 mm by 100 mm.

60. The method of claim 58, wherein imaging morphological features of the contacted cells in step (f) further comprises:
(i) using a software program that processes images of morphological features of each cell in the field of view;
(ii) moving the field of view digitally;
(iii) using a software program to process images of morphological features of each cell in the next field of view and repeating steps (ii) and (iii) until the entire substrate area is analyzed;
(iv) digitally combining all images obtained to generate a single image covering the entire selected substrate area; and
(v) generating from the data obtained for the entire substrate area the morphological features of each substrate-adhered cell to detect the biomarker-morphological profile of each cell.

61. The method of claim 1, wherein the method further comprises the steps of (d)(2) removing a first plurality of functionalized nanoparticles; and (d)(3) contacting the cells with a second plurality of functionalized nanoparticle species.

62. The method of claim 61, wherein the method further comprises after contacting the cells with a second plurality of functionalized nanoparticle species, (d)(4) removing the second plurality of functionalized nanoparticles; (d)(5) contacting the cells with a third plurality of functionalized nanoparticle species; and, optionally (d)(6) removing the previous plurality of functionalized nanoparticle species; and (d)(7) contacting the cells with a next plurality of functionalized nanoparticles, and optionally, repeating steps (d)(6) and (d)(7) an number of times from none to ten.

63. The method of claim 61 or 62, wherein the removing a first plurality of functionalized nanoparticles is achieved by cleaving a cleavable linker between each species of functionalized nanoparticle and each species of a functionalized nanoparticle-associated biomarker-binding moiety.

64. The method of claim 61, wherein the removing a first plurality of functionalized nanoparticles is achieved by displacing the first plurality of functionalized nanoparticles from the biomarker binding moieties.

65. The method of claim 62, wherein the removing the second or previous plurality of functionalized nanoparticles is achieved by displacing the second or previous plurality of functionalized nanoparticles from the biomarker binding moieties.

66. The method of claim 63, wherein the linker between each nanoparticle species in the first plurality of functionalized nanoparticles and its respective biomarker binding moiety comprises a first oligonucleotide bound to a first nanoparticle species and a second oligonucleotide bound to its respective biomarker binding moiety, wherein the second oligonucleotide comprises a portion complementary to a portion of the first oligonucleotide and hybridization of the first oligonucleotide to the second oligonucleotide forms a linker comprising a double-stranded nucleic acid.

67. The method of claim 66, wherein the linker between each functionalized nanoparticle species in the second or next plurality of functionalized nanoparticles and its respective biomarker binding moiety comprises a second or next oligonucleotide bound to a second or next functionalized nanoparticle species, respectively, and a third or next oligonucleotide bound to its respective biomarker binding moiety, wherein the third or next oligonucleotide comprises a portion complementary to a portion of the second or next oligonucleotide and hybridization of the second or next oligonucleotide to the third or next oligonucleotide forms a linker comprising a double-stranded nucleic acid.

68. The method of claim 64, wherein each functionalized nanoparticle species is displaced from its respective biomarker binding moiety by binding of a third oligonucleotide to the first oligonucleotide wherein the hybrid formed by hybridization of the third oligonucleotide and the first oligonucleotide exhibits a melting temperature higher than the melting temperature of the double-stranded nucleic acid formed by hybridization of the first and second oligonucleotide.

69. The method of claim 68, where the steps (b)-(d) are repeated up to one, two, three, four, five, six, seven, eight, nine, ten, fifteen, twenty, thirty, forty, or fifty or more times.

70. The method of claim 65, wherein after displacing the second or previous plurality of functionalized nanoparticles from the biomarker binding moieties, the following steps are performed:
(i) the biomarker-binding moieties of the functionalized nanoparticles which bound to the cell are associated with the biomarker binding moiety functionalized-nanoparticle is classified, and
(ii) the cells are contacted with a next plurality of nanoparticles functionalized with different biomarker binding moieties, and each functionalized nanoparticle species of the next plurality of nanoparticles are functionalized with different biomarker binding moieties that binds to a biomarker which is suspected of being associated with samples in which the first biomarker is present.

71. The method of claim 1, wherein the cells may be the same type or different types from each other.

72. The method of claim 71, wherein the cells are different types from each other.

73. The method of claim 72, wherein the cells can be from different conditions.

74. The method of claim 73, wherein the different conditions are selected from: having a hematological cancer, non-malignant hematological disorder, solid tumor, bladder disease, liver disease, kidney disease, or infectious disease.

75. The method of claim 74, wherein the different conditions are having a different type of solid tumor.

76. The method of claim 1, wherein the biomarker-binding moiety is anti-CD45, and the biomarker signature obtained is the white blood cell count.

77. The method of claim 1, wherein the cells are live.

78. The method of claim 1, wherein the cells are fixed.

79. The method of claim 78, wherein the cells are fixed with formaldehyde.

80. The method of claim 1, wherein the substrate is selected from: glass silica, clear polymer, gold, or alumina.

81. The method of claim 1, wherein the substrate is functionalized.

82. The method of claim 81, wherein the substrate functionalization is patterned.

83. The method of claim 82, wherein the functionalization is a silane-linked cell biomarker, polymer-linked cell biomarker, silane-linked amine, silane-linked carboxylic acid, polymer-linked amine, polymer-linked carboxylic acid, polyethylene glycol (PEG), amino-functionalized dextran, gold, silver, alumina, or glass silica.

84. The method of claim 1, wherein the sample is from blood, bone marrow, fine needle aspirate, or tissue.

85. The method of claim 84, wherein the tissue sample is FFPE (formalin-fixed, paraffin-embedded) tissue samples.

86. The method of claim 84, wherein when the sample is tissue, the optical contrast agent is H&E (hematoxylin and eosin) stain.

87. A method for detecting the biomarker-morphological profile of a cell, the method comprising:
(a) providing a sample comprising cells from a subject;
(b) contacting the cells with one or a plurality of functionalized nanoparticle species, each functionalized nanoparticle species comprising a biomarker-binding moiety, and forming nanoparticle-cell complexes through binding of the nanoparticle species comprising a biomarker-binding moiety to its respective biomarker;
(c) adhering the functionalized nanoparticle-cell complexes to a substrate;
(d) illuminating the functionalized nanoparticle-cell complexes with non-evanescent light and detecting the resonant light scattering from each observed complexed nanoparticle, to obtain a biomarker signature of each observed cell;
(e) contacting the substrate-adhered cells with an optical contrast agent;
(f) imaging morphological features of the contacted cells; and
(g) associating the morphological features of the contacted cells with the biomarker signature of each substrate-adhered cell to detect the biomarker-morphological profile of each cell.

88. The method of claim 87, wherein the non-evanescent light is transmitted light.

89. A method for detecting the biomarker-morphological profile of a cell, the method comprising:
(a) providing a sample comprising cells from a subject;
(b) adhering the cells to a substrate;
(c) contacting the substrate-adhered cells with an optical contrast agent;
(d) imaging morphological features of the contacted cells;
(e) converting the optical contrast agent to a colorless form;
(f) contacting the cells with one or a plurality of functionalized nanoparticle species, each functionalized nanoparticle species comprising a biomarker-binding moiety to form nanoparticle-cell complexes;
(g) illuminating the nanoparticle-cell complexes with evanescent light and detecting the resonant light scattering from each observed complexed nanoparticle, to obtain a biomarker signature of each observed cell; and
(h) associating the morphological features of the contacted cells with the biomarker signature of each substrate-adhered cell to detect the biomarker-morphological profile of each cell.

90. The method of claim 89, wherein the optical contrast agent is a leuco dye.

91. The method of claim 90, wherein the leuco dye is methylene blue, methylene green, red leuco dye, crystal violet, phenolphthalein, or thymolphthalein.

92. The method of claim 90, wherein the leuco dye is converted to a colorless form by the addition of one or more electrons to the dye.

93. The method of claim 91, wherein the leuco dye is converted to a colored form by the removal of one or more electrons from the dye.

94. A kit for the detection of a biomarker signature, the combination comprising a plurality of biomarker-binding moiety functionalized nanoparticle species.

95. The kit of claim 94, wherein the functionalized nanoparticle species further comprise:
(a) a nanoparticle species bound to a first oligonucleotide; and
(b) a biomarker-binding moiety bound to a second oligonucleotide,
wherein the first oligonucleotide is complementary to a portion of the second oligonucleotide, and the first and second oligonucleotide form a hybridized duplex.

96. The kit of claim 94, wherein the functionalized nanoparticle species further comprise:
(a) a nanoparticle functionalized with a first oligonucleotide;
(b) a biomarker-binding moiety functionalized with a second oligonucleotide; and
a third oligonucleotide,
wherein the first oligonucleotide is complementary to a portion of the third oligonucleotide, the second oligonucleotide is complementary to a separate portion of the third oligonucleotide, and the first and second oligonucleotides form a hybridized duplex to the third oligonucleotide.

97. A kit for the detection of a biomarker morphological profile, the combination comprising a plurality of functionalized nanoparticle species and an optical contrast agent.

98. The kit of any one of claim 94 or 97, wherein the plurality of functionalized nanoparticle species comprise a mixture.

99. The kit of any one of claim 94 or 97, wherein the plurality of functionalized nanoparticle species are segregated before use.

100. A kit comprising a plurality of functionalized nanoparticle species, and a mountant.

101. The kit of claim 100, wherein the mountant has a refractive index of within 0.1 of the refractive index of fixed cells.

102. The method of claim 56, wherein the mountant has a refractive index of within 0.1 of the refractive index of fixed cells.

103. The kit of any one of claims 100-102, further comprising an optical contrast agent.

104. A method for increasing the loading amount of a functionalized nanoparticle onto a cell by using an external force to increase the local concentration of the nanoparticles and cells.

105. The method of claim 104, wherein the external force is a centrifugal, electrical or magnetic force.

106. The method of claim 1, wherein the plurality of nanoparticles exhibit a peak resonance wavelength of the nanoparticle plasmon resonance between 400 to 900 nm.

107. A method for detecting functionalized nanoparticle cell complexes, the method comprising:
(a) providing a sample comprising cells from a subject;
(b) contacting the cells with one or a plurality of functionalized nanoparticle species, each functionalized nanoparticle species comprising a biomarker-binding moiety, wherein an external force is used to accelerate the formation of nanoparticle-cell complexes through binding of the nanoparticle species comprising a biomarker-binding moiety to its respective biomarker;
(c) adhering the functionalized nanoparticle-cell complexes to a substrate;
(d) detecting the functionalized nanoparticle cell complexes by illuminating the nanoparticle-cell complexes with evanescent light and detecting the resonant light scattering from each observed functionalized nanoparticle cell complex, to obtain a biomarker signature of each observed cell; and
(e) associating the biomarker signature of each substrate-adhered cell to a known disease, condition, or state of a cell exhibiting substantially the same biomarker signature to identify the disease, condition, or state of the cell from a subject.

108. A homogeneous assay for detecting functionalized nanoparticle cell complexes, the assay comprising:
(a) providing a sample comprising cells from a subject;
(b) contacting the cells with one or a plurality of functionalized nanoparticle species, each functionalized nanoparticle species comprising a biomarker-binding moiety, and forming functionalized nanoparticle-cell complexes through binding of the nanoparticle species comprising a biomarker-binding moiety to its respective biomarker;
(c) adhering the functionalized nanoparticle-cell complexes to a substrate;
(d) illuminating the functionalized nanoparticle-cell complexes with evanescent light and detecting the resonant light scattering from each observed complexed nanoparticle, to obtain a biomarker signature of each observed cell,
wherein unbound functionalized nanoparticles are not removed from the field of view.

109. A method for detecting functionalized nanoparticle cell complexes, the method comprising:
(a) providing a sample comprising cells from a subject;
(b) contacting cells which have been fixed with one or a plurality of functionalized nanoparticle species, each functionalized nanoparticle species comprising a biomarker-binding moiety, and forming nanoparticle-cell complexes through binding of the nanoparticle species comprising a biomarker-binding moiety to its respective biomarker;
(c) adhering the nanoparticle-cell complexes to a substrate, wherein the adhered functionalized nanoparticle-cell complexes are placed in contact with a mountant, wherein the refractive index of the mountant is within about 0.1 of the refractice index of the fixed cells;
(d) detecting the functionalized nanoparticle cell complexes by illuminating the nanoparticle-cell complexes with evanescent light and detecting the resonant light scattering from each observed functionalized nanoparticle cell complex, to obtain a biomarker signature of each observed cell; and
(e) associating the biomarker signature of each substrate-adhered cell to a known disease, condition, or state of a cell exhibiting substantially the same biomarker signature to identify the disease, condition, or state of the cell from a subject.

110. The method of claim 109, wherein the mountant has an refractive index of from 1.51 to 1.54.

111. A method for detecting functionalized nanoparticle cell complexes to obtain a biomarker signature, the method comprising:
(a) providing a sample comprising cells from a subject;
(b) contacting the cells with one or a plurality of functionalized nanoparticle species, each functionalized nanoparticle species comprising a biomarker-binding moiety, and forming nanoparticle-cell complexes through binding of the nanoparticle species comprising a biomarker-binding moiety to its respective biomarker;
(c) adhering the functionalized nanoparticle-cell complexes to a substrate;
(d) illuminating the functionalized nanoparticle-cell complexes with evanescent light and detecting the resonant light scattering from each observed complexed nanoparticle, to obtain a biomarker signature of each observed cell; and
(e) associating the biomarker signature of an imaged cell from the subject with a biomarker signature of a reference cell exhibiting substantially the same biomarker signature as the imaged cell biomarker signature, wherein a diagnostic concordance has been established between the reference cell biomarker signature and a disease, disorder, condition or state of the reference subject.

112. The method of any one of claim 1, 87, 89, 104, 108, 109, or 111, wherein the steps of providing a sample comprising cells from a subject, contacting the cells with one or a plurality of functionalized nanoparticle species, and adhering the functionalized nanoparticle-cell complexes to a substrate, are performed by an automated liquid handling system.

113. The method of any one of claim 107, 108, 109, or 111, further comprising obtaining and storing the positional information for each observed cell is stored.

114. The method of any one of claim 1, 87, or 89, further comprising obtaining and storing the positional information for each observed and imaged cell is stored.

115. The method of claim 1, wherein the nanoparticle preparations are of a narrow size distribution, such that an individual nanoparticle preparation has a scattering spectrum whose full-width half maximum ranges from 5 to 150 nm.

116. The method of claim 1, wherein the functionalized nanoparticle is present on the cell surface.

117. The method of claim 1, wherein the functionalized nanoparticle is present within the cell.
